SEQUENCE LISTING (41 sequences)

SEQ ID NO: 1 - DNA, 1234 bp, Homo sapiens
ggcgacaacaaaacagattactttgaagggactagaaggaggacacagggtcctggaggaaggcagctggcaagttacagagcagaaagactttgcacctctggctccagggagctcacaggcaggggctggtccagcccagaggcgatgaagacagtgacgatgatgatggtggtggagatgcaggcgctgactcagctgtctctctgtgactgtctttgtgacagtctcattgttgttgttactaagatggcttccctcagaggacggacacttggagtagtggcaggttcggccacccctagagacgatggcggtggggaccggggcagccccctggccccctgggctcacccccctcctgcgccaccccgggccgtgccgtgccaagccggccaccagagggcgcgccaggtcgcggcatcccggctccccgtcggcctggggccggcgggcggggctcggctgtctcctactgaggcctcgcacttcctctcttgtacttctgtgtttcttggagagctgctccttgaagatactgctgcatgtgggtctctgtgggactgtcagttaagagcctccactggcctcacccacccccagagggaatacatgtcctcggctgagcccattgggctttctctcctgaccaatcagattatttctcttctgactcaggttctgagagctgtgttgtctgcgtgcacatgggtgagccggaaaggagacctgcagagaatgaaacagacacataaaggaaagcctcccagcagcatggctttcaccggcaagttcgagatggagagtgagaagaattatgatgagttcatgaagctccttgggatctccagcgatgtaatcgaaaaggcccgcaacttcaagatcgtcacggaggtgcagcaggatgggcaggacttcacttggtcccagcactactccgggggccacaccatgaccaacaagttcactgttggcaaggaaagcaacatacagacaatggggggcaagacgttcaaggccactgtgcagatggagggcgggaagctggtggtgaatttccccaactatcaccagacctcagagatcgtgggtgacaagctggtggaggtctccaccatcggaggcgtgacctatgagcgcgtgagcaagagactggcctaagcagccaggcccggcccagggagctacaaacccaccaataaaactgatataaggacaaaaaaaaaaaaaaaaa

SEQ ID NO: 2 - DNA, 627 bp, Homo sapiens
atgaagacagtgacgatgatgatggtggtggagatgcaggcgctgactcagctgtctctctgtgactgtctttgtgacagtctcattgttgttgttactaagatggcttccctcagaggacggacacttggagtagtggcaggttcggccacccctagagacgatggcggtggggaccggggcagccccctggccccctgggctcacccccctcctgcgccaccccgggccgtgccgtgccaagccggccaccagagggcgcgccaggtcgcggcatcccggctccccgtcggcctggggccggcgggcggggctcggctgtctcctactgaggcctcgcacttcctctcttgtacttctgtgtttcttggagagctgctccttgaagatactgctgcatgtgggtctctgtgggactgtcagttaagagcctccactggcctcacccacccccagagggaatacatgtcctcggctgagcccattgggctttctctcctgaccaatcagattatttctcttctgactcaggttctgagagctgtgttgtctgcgtgcacatgggtgagccggaaaggagacctgcagagaatgaaacagacacataaaggaaagcctcccagcagc

SEQ ID NO: 3 - DNA, 739 bp, Homo sapiens
cagcgacaacaaaacagattactttgaagggactagaaggaggacacagggtcctggaggaaggcagctggcaagttacagagcagaaagactttgcacctctggctccagggagctcacaggcaggggctggtccagcccagaggcgatgaagacagtgacgatgatgatggtggtggagatgcaggcgctgactcaggttctgagagctgtgttgtctgcgtgcacatgggtgagccggaaaggagacctgcagagaatgaaacagacacataaaggaaagcctcccagcagcatggctttcaccggcaagttcgagatggagagtgagaagaattatgatgagttcatgaagctccttggggatctccagcgatgtaatcgaaaaggcccgcaacttcaagatcgtcacggaggtgcagcaggatgggcaggacttcacttggtcccagcactactccgggggccacaccatgaccaacaagttcactgttggcaaggaaagcaacatacagacaatggggggcaagacgttcaaggccactgtgcagatggagggcgggaagctggtggtgaatttccccaactatcaccagacctcagagatcgtgggtgacaagctggtggaggtctccaccatcggaggcgtgacctatgagcgcgtgagcaagagactggcctaagcagccaggcccggcccagggagctacaaacccaccaataaaactgatataaggac

SEQ ID NO: 4 - DNA, 571 bp, Homo sapiens
gaagaagtggggtgacttaggggctgagcctcagcaactgggagagtttataagctggatagcagacccctcagcaccacccattctcctcatccctctgctctctggcctccagcctcccagcagcatggctttcaccggcaagttcgagatggagagtgagaagaattatgatgagttcatgaagctccttggggatctccagcgatgtaatcgaaaaggcccgcaacttcaagatcgtcacggaggtgcagcaggatgggcaggacttcacttggtcccagcactactccgggggccacaccatgaccaacaagttcactgttggcaaggaaagcaacatacagacaatggggggcaagacgttcaaggccactgtgcagatggagggcgggaagctggtggtgaatttccccaactatcaccagacctcagagatcgtgggtgacaagctggtggaggtctccaccatcggaggcgtgacctatgagcgcgtgagcaagagactggcctaagcagccaggcccggcccagggagctacaaacccaccaataaaactgatataaggac

SEQ ID NO: 5 - DNA, 607 bp, Homo sapiens
atgaagacagtgacgatgatgatggtggtggagatgcaggcgctgactcaggttctgagagctgtgttgtctgcgtgcacatgggtgagccggaaaggagacctgcagagaatgaaacagacacataaaggaaagcctcccagcagcatggctttcaccggcaagttcgagatggagagtgagaagaattatgatgagttcatgaagctccttgggatctccagcgatgtaatcgaaaaggcccgcaacttcaagatcgtcacggaggtgcagcaggatgggcaggacttcacttggtcccagcactactccgggggccacaccatgaccaacaagttcactgttggcaaggaaagcaacatacagacaatggggggcaagacgttcaaggccactgtgcagatggagggcgggaagctggtggtgaatttccccaactatcaccagacctcagagatcgtgggtgacaagctggtggaggtctccaccatcggaggcgtgacctatgagcgcgtgagcaagagactggcctaagcagccaggcccggcccagggagctacaaacccaccaataaaactgatataaggacaaaaaaaaaaaaaaaaa

SEQ ID NO: 6 - DNA, 147 bp, Homo sapiens
atgaagacagtgacgatgatgatggtggtggagatgcaggcgctgactcaggttctgagagctgtgttgtctgcgtgcacatgggtgagccggaaaggagacctgcagagaatgaaacagacacataaaggaaagcctcccagcagc

SEQ ID NO: 7 - protein, 176 aa, Homo sapiens
MKTVMMMVVEMQALTQVLRAVLSACTWVSRKGDLQRMKQTHKGKPPSSMAFTGKFEMESEKNYDEFMKLLGISSDVIEKARNFKIVTEVQQDGQDFTWSQHYSGGHTMTNKFTVGKESNIQTMGGKTFKATVQMEGGKLVVNFPNYHQTSEIVGDKLVEVSTIGGVTYERVSKRLA

SEQ ID NO: 8 - protein, 128 aa, Homo sapiens
MAFTGKFEMESEKNYDEFMKLLGISSDVIEKARNFKIVTEVQQDGQDFTWSQHYSGGHTMTNKFTVGKESNIQTMGGKTFKATVQMEGGKLVVNFPNYHQTSEIVGDKLVEVSTIGGVTYERVSKRLA

SEQ ID NO: 9 - protein, 177 aa, Homo sapiens
MKTVTMMMVVEMQALTQVLRAVLSACTWVSRKGDLQRMKQTHKGKPPSSMAFTGKFEMESEKNYDEFMKLLGISSDVIEKARNFKIVTEVQQDGQDFTWSQHYSGGHTMTNKFTVGKESNIQTMGGKTFKATVQMEGGKLVVNFPNYHQTSEIVGDKLVEVSTIGGVTYERVSKRLA

SEQ ID NO: 10 - DNA, 27 bp, Artificial Sequence
  Description of Artificial Sequence Synthetic primer
ccacccattctcctcatccctctgctc

SEQ ID NO: 11 - DNA, 25 bp, Artificial Sequence
  Description of Artificial Sequence Synthetic primer
accaagtgaagtcctgcccatcctg

SEQ ID NO: 12 - DNA, 24 bp, Artificial Sequence
  Description of Artificial Sequence Synthetic primer
acatgggtgagccggaaaggagac

SEQ ID NO: 13 - DNA, 28 bp, Artificial Sequence
  Description of Artificial Sequence Synthetic primer
ccggagtagtgctgggaccaagtgaagt

SEQ ID NO: 14 - DNA, 20 bp, Artificial Sequence
  Description of Artificial Sequence Synthetic primer
caagactggagacaaagtgg

SEQ ID NO: 15 - DNA, 20 bp, Artificial Sequence
  Description of Artificial Sequence Synthetic primer
aatctgcagacagacactgg

SEQ ID NO: 16 - protein, 24 aa, Artificial Sequence
  Description of Artificial Sequence Synthetic peptide
CTWVSRKGDLQRMKQTHKGKPPSS

SEQ ID NO: 17 - RNA, 21 nt, Homo sapiens
gcccgcaacuucaagaucguc

SEQ ID NO: 18 - RNA, 21 nt, Homo sapiens
gacgaucuugaaguugcgggc

SEQ ID NO: 19 - DNA, 21 bp, Artificial Sequence
  Description of Artificial Sequence Synthetic oligonucleotide
gcccgcaacttcaagatcgtc

SEQ ID NO: 20 - DNA, 21 bp, Artificial Sequence
  Description of Artificial Sequence Synthetic oligonucleotide
gacgatcttgaagttgcgggc

SEQ ID NO: 21 - DNA, 1642 bp, Homo sapiens
ctagtggatgaatctgggtaagggaactgtctggggacagacagggctggaagggaggggaacaaaggacattggatttggggcccagatgaagttgaagatatgctggagggaggagtaaatgaatggcaggaggaggaagtgctggtcaaagagaacccctgaccttggaatttcagaggaagtgtgatgctaggatgggaggcagggacgtgatggtgggaggatggctgaggtgcgtggagggcaccattgattacagggactaagaaacagaggccacatgtcagtgggtccttcagctggaggctgaagtggtggtgacaggaataaagatgaagacagctgtgagccactctgaccagtgcagaaagtgaagtagagcttcctcttcaaagggactttccttcccgtctaattaggaataaacagtaacttctcttagaagcaaaatttattcaaagacctgtgctaacattcttagaaatctgctagccataataaagaaatcaatgtactttgtgttcttagcacccacattttagcctagatatttgccctggcgtgcttatactgatccaagcaagcagtaggtcatagcctgttcctcttccttattcggaggtgtttttacctttctcagcattccacaggttgcttcctccttcctttgttctcctctgcctttgcctctttcggaaagttctaaatgactagccaatcgggacaagtatagaatgtgaggtacccttccagccaacggaaactggacacagcagtagggtggatgcatcaggttataaatgaccctgtctcctttgttcgtgtgtgctcttgcgagaattactgatagtgatggcaccctttctgcagaaagtaaactagccttgctgagagatcctttgttttttatgacaccaagcacctgttcccaacaccagtgagggccacctccaagaagctggcaaaagagaggaacaagaagagttgtgggcaggaaagattgatgtaatgaacccacaggtggtaatgtgtttggaagaagggaggagaaagggattgcaagagccaggtgtggtggctcacacctgtaagctcaacactctgggaggccgaggagggcggatcacttgagctcaggagtttgagaccagtctgggcaacatggtgagatcccgtctgtaccaaaaatacaaaaacttagctgggcctggtggtgcccatcggtggtcccagctacttgggaggttgaggtggaagttaagctggagagtggggcattaggggggtgggggttgcagtgagccgagattgggccactgcactctaacctgggtgacatagtgaaaaaaaaaaaaagattgcaagagagtgaactgacctcccaggccctgatctacagggtgtgtgagaaaattaaaaacaggtggcaccggcctgtagtcccagctactcaggagaaagaggcgggaggatcgcttgagcccaggaggctgggtagagcctgcagagagctatgattacactactgcactccaggctgggtgaccctgtttccaacaacaacagcgacaacaaaacagattactttgaagggactagaaggaggacacagggtcctggaggaaggcagctggcaagt

SEQ ID NO: 22 - DNA, 15 bp, Homo sapiens
aaagggactttcctt

SEQ ID NO: 23 - DNA, 14 bp, Homo sapiens
ccaacaacaacagc

SEQ ID NO: 24 - DNA, 14 bp, Homo sapiens
  modified_base (1): a, c, g, t, unknown or other
nggggamtttccnn

SEQ ID NO: 25 - DNA, 10 bp, Homo sapiens
gggamttycc

SEQ ID NO: 26 - DNA, 15 bp, Homo sapiens
aaagcgactttcctt

SEQ ID NO: 27 - DNA, 28 bp, Artificial Sequence
  Description of Artificial Sequence Synthetic primer
acattatattttcttgccaagtagagga

SEQ ID NO: 28 - DNA, 19 bp, Artificial Sequence
  Description of Artificial Sequence Synthetic primer
acttgccagctgccttcct

SEQ ID NO: 29 - RNA, 21 nt, Artificial Sequence
  Description of Artificial Sequence Synthetic oligonucleotide
ugaaacagacacauaaagguu

SEQ ID NO: 30 - RNA, 21 nt, Artificial Sequence
  Description of Artificial Sequence Synthetic oligonucleotide
ccuuuaugugucuguuucauu

SEQ ID NO: 31 - RNA, 21 nt, Artificial Sequence
  Description of Artificial Sequence Synthetic oligonucleotide
aggagaccugcagagaauguu

SEQ ID NO: 32 - RNA, 21 nt, Artificial Sequence
  Description of Artificial Sequence Synthetic oligonucleotide
cauucucugcaggucuccuuu

SEQ ID NO: 33 - RNA, 21 nt, Artificial Sequence
  Description of Artificial Sequence Synthetic oligonucleotide
gacagugacgaugaugauguu

SEQ ID NO: 34 - RNA, 21 nt, Artificial Sequence
  Description of Artificial Sequence Synthetic oligonucleotide
caucaucaucgucacugucuu

SEQ ID NO: 35 - RNA, 21 nt, Artificial Sequence
  Description of Artificial Sequence Synthetic oligonucleotide
cugggagaguuuauaagcuuu

SEQ ID NO: 36 - RNA, 21 nt, Artificial Sequence
  Description of Artificial Sequence Synthetic oligonucleotide
agcuuauaaacucucccaguu

SEQ ID NO: 37 - RNA, 21 nt, Artificial Sequence
  Description of Artificial Sequence Synthetic oligonucleotide
agcaacugggagaguuuauuu

SEQ ID NO: 38 - RNA, 21 nt, Artificial Sequence
  Description of Artificial Sequence Synthetic oligonucleotide
auaaacucucccaguugcuuu

SEQ ID NO: 39 - RNA, 21 nt, Artificial Sequence
  Description of Artificial Sequence Synthetic oligonucleotide
gagaguuuauaagcugggaua

SEQ ID NO: 40 - RNA, 21 nt, Artificial Sequence
  Description of Artificial Sequence Synthetic oligonucleotide
uaucccagcuuauaaacucuc

SEQ ID NO: 41 - protein, 6 aa, Artificial Sequence
  Description of Artificial Sequence Synthetic 6xHis tag
HHHHHH

(19) United States
(12) Patent Application Publication
(10) Pub. No.: US 2009/0074785 A1
(43) Pub. Date: Mar. 19, 2009

(54) Compositions and methods for treatment of colorectal cancer

(76) Inventors: Jeffrey W. Smith, San Diego, CA (US); Changming Fang, San Diego, CA (US)

Correspondence Address: Biotechnology Law Group;c/o Portfolioip, P.O. Box 52050, Minneapolis, MN, 55402, US

(21) Appl. No.: 12/009,173

(22) Filed: Jan. 16, 2008

Related U.S. Application Data
(60) Provisional application No. 60/880,932, filed on Jan. 16, 2007.

(51) Int. Cl. A61K 39/395; A61K 31/7105; A61K 31/575; A61P 35/00; G01N 33/53; A61K 39/00

(52) U.S. Cl. 424/141.1; 514/44; 424/277.1; 514/182; 435/7.1

(57) ABSTRACT

We have identified a new variant of ileal bile acid binding protein (IBABP), designated IBABP-L, which is a biomarker for colorectal cancer. The transcript for IBABP-L arises from an alternative start site and includes three exons that are absent in IBABP. IBABP-L also shares part of a fourth exon with IBABP. The protein encoded by IBABP-L contains a deduced 49 residue N-terminal sequence that is not found in the IBABP protein. The present invention provides, for example, compositions and methods for diagnosing and treating colorectal cancer.

### STATEMENT OF GOVERNMENT RIGHTS

The invention was supported, at least in part, by a grant from the Government of the United States of America (grants no. R21CA 116329 and R01 CA 108959 from the National Institutes of Health). The Government may have certain rights to the invention.

### BACKGROUND

1. Technical Field

The present invention relates to methods and compositions for the treatment of colon cancer.

2. Background Information

Colorectal cancer is the third most prevalent malignancy in the United States with approximately 145,000 new diagnoses and 56,000 deaths estimated for 2005 [Cancer Facts and Figures 2005, Surveillance Research (Washington, D.C.: American Cancer Society, Inc., 2005). The most common non-invasive test for colorectal cancer is the fecal occult blood test (FOBT), which has been used for more than thirty years. Unfortunately, the sensitivity of the FOBT remains around 50% and may not detect early malignancy since not all carcinomas shed blood (Agrawal and Syngal, Curr. Opin. Gastroenterol. 21:59-63, 2005). Because of the high number of false-positives associated with FOBT, colonoscopy and sigmoidoscopy remain the gold standard for detecting colon cancer (Smith et al., CA Cancer J. Clin. 55:31-44, 2005). These invasive exams are expensive, require highly trained staff, are uncomfortable, and raise the risk of bowel perforation and possible mortality (Davies et al., Nat. Rev. Cancer 5:199-209, 2005). Consequently, there is still a great need for new molecular markers of and diagnostic tests for colorectal cancer.

One might expect to find alterations in the expression of proteins associated with known risk factors for colon cancer, like bile acids, which were recognized as carcinogens as early as 1940 (Cook et al., Nature 145:627, 1940) and have since been strongly linked with the incidence of colon cancer (Wunder and Reddy, J. Natl. Cancer Inst. 50:1099-1106, 1973; Crowther et al., Br. J. Cancer 34:191-198, 1976; Reddy et al., Cancer 42:2832-2838, 1978; Jensen et al., Nutr. Cancer 4:5-19, 1982; Hill et al., Nutr. Cancer 4:67-73, 1982; Domellof et al., Nutr. Cancer 4:120-127, 1982). Only a few studies have examined proteins in the bile-acid response pathway as potential biomarkers in colorectal cancer (DeGottardi et al., Dig. Dis. Sci. 49:982-989, 2004).

One of the proteins involved in bile acid homeostasis is ileal bile acid binding protein (IBABP), a 14 kDa cytoplasmic protein that is part of the fatty acid binding protein (FABP) family. IBABP is encoded by the fabp6 gene on chromosome 5 (Fujita et al., Eur. J. Biochem. 233:406-423, 1995); IBABP is unique among this protein family because it binds to bile acids instead of fatty acids (Thompson et al., Mol. Cell. Biochem. 192:9-16, 1999). Moreover, bile acids induce the expression of IBABP (Kanda et al., Biochem. J. 330(Pt. 1):261-265, 1998) by binding nuclear farnesoid-X receptor (FXR) in the intestine (Makishima et al., Science 284:1362-1365, 1999; Wang et al., Mol. Cell. 3:543-553, 1999; Parks et al., Science 284:1365-1368, 1999), thereby activating a bile acid responsive element in the IBABP promoter (Grober et al., J. Biol. Chem. 274:29749-29754, 1999). Based on these properties, and its localization in intestinal epithelium, IBABP may be involved in bile acid transport and buffering activities central to control of cholesterol homeostasis (Fuchs, Am. J. Physiol. Gastrointest. Liver Physiol. 284:G551-G557, 2003).

One pathway receiving increased attention in colorectal cancer is that controlled by the NF-κB transcription factor. In a resting state, the p50 and p65 subunits of NF-κB form a heterotrimeric complex with IκBα that sequesters them in the cytoplasm. In response to various stimuli, IκBα is degraded and the p50-p65 heterodimer is translocated to the nucleus where it binds specific regulatory elements and drives gene expression (Bharti and Aggarwal, Biochem. Pharmacol. 64:883-888, 2002). NF-κB is up-regulated in colorectal adenoma and adenocarcinoma (Karin, Mol. Carcinog. 45:355-361, 2006; Yu et al., Eur. J. Surg. Oncol. 31:386-392, 2005; Lind et al., Surgery 130:363-369, 2001; Kojima et al., Anticancer Res. 24:675-681, 2004) and controls the expression of many colorectal cancer-linked genes, including cyclooxygenase-2 (COX-2) and Bcl-2 (Bharti and Aggarwal, Biochem. Pharmacol. 64:883-888, 2002). NF-κB is also believed to be involved in the onset of colorectal cancer (Bharti and Aggarwal, Biochem. Pharmacol. 64:883-888, 2002), in the inflammatory process associated with colorectal cancer (Itzkowitz and Yio, Am. J. Physiol. Gastroinest. Liver Physiol. 287:G7-17, 2004), and in the development of resistance of colorectal cancer to chemotherapy (Cusack, Ann. Surg. Oncol. 10:852-862, 2003; Luo et al., J. Clin. Invest. 115:2625-2632, 2005).

There is a need for effective treatments for colorectal cancer in patients. The present invention meets this and other needs.

### SUMMARY OF THE INVENTION

We have discovered an unanticipated link between NF-κB and bile acids. We have identified a variant of IBABP that arises from an alternative transcription start site. Unlike IBABP, which is transcribed by the farnesoid X receptor/bile acid receptor (FXR), the new variant, called IBABP-L, is regulated by an NF-κB binding site in a distal promoter. IBABP-L contains 49 amino acids at its N-terminus that are absent in IBABP. More significantly, the transcript for IBABP-L is up-regulated in all stages of colorectal adenocarcinoma. In fact, we show that the up-regulation of IBABP in colorectal cancer reported in prior studies (DeGottardi et al., Dig. Dis. Sci. 49:982-989, 2004; Ohmachi et al., Clin. Cancer Res. 12:5090-5095, 2006) can be attributed to the up-regulation of IBABP-L, while the expression of the previously defined form of IBABP is unchanged in colorectal cancer. Most significantly, IBABPP-L is necessary for the survival of colon cancer cells in the presence of secondary bile acids. These observations provide an important mechanistic link between bile acids, NF-κB and colorectal cancer that can be exploited for therapeutic benefit.

According to one embodiment of the invention, methods are provided for reducing the growth or survival of a colorectal cancer cell comprising contacting the cell with an effective amount of a composition comprising a substance that reduces bile-acid binding by IBABP-L.

In one such method, the substance reduces IBABP-L polypeptide levels in the cell without reducing IBABP polypeptide levels. In another such method, the substance inhibits IBABP-L gene expression.

In another such method, the substance is a polynucleotide, including, but not limited to, one or more of the following: an siRNA (as defined below), an antisense polynucleotide, or a ribozyme.

In another such method, the polynucleotide comprises a promoter that is expressible in the colorectal cancer cell and that is operably linked to a sequence encoding a polynucleotide that, when expressed, reduces levels of a polypeptide selected from the group consisting of IBAB-L, a enzyme of metabolism, a protein essential for cell-cycle progression, a protein that inhibits apoptosis, a protein involved in growth regulatory signal transduction, and a protein involved in bile acid transport out of colorectal cancer cells.

In another such method, the polynucleotide comprises a promoter that is expressible in the colorectal cancer cell and that is operably linked to a sequence encoding an siRNA, an antisense polynucleotide, or a ribozyme.

In another such method, the polynucleotide comprises a promoter that is expressible in the colorectal cancer cell and that is operably linked to a sequence encoding a polypeptide selected from the group consisting of a pro-apoptotic protein, a tumor suppressor protein, a protein that inhibits cell cycle progression, and a protein involved in the delivery of toxic secondary bile acids into the cytoplasm of colorectal cancer cells.

In another such method, the substance inhibits transcriptional activation of IBABP-L gene expression.

In another such method, the composition comprises a member of the group consisting of a bile acid, a chemotherapeutic drug, a non-steroidal anti-inflammatory drug; a vaccine comprising autologous tumor cells, a vaccine comprising a tumor-associated antigen, an monoclonal antibody directed against a tumor antigen, a recombinant construct for gene correction, a virus-directed enzyme-prodrug treatment, and a matrix metalloproteinase inhibitor.

In another such method, the composition comprises a bile acid selected from the group consisting of cholic acid and deoxycholic acid.

In another such method, the composition causes apoptosis of the colorectal cancer cell in the presence of a bile acid.

According to another embodiment of the invention, methods are provided for treating colorectal cancer in a patient in need of such treatment comprising administering to the patient an effective amount of a composition that reduces bile-acid binding by IBABP-L, as further specified above.

According to another embodiment of the invention, compositions are provided that comprise a polynucleotide selected from the group consisting of: an expression vector comprising an IBABP-L promoter operably linked to a sequence that, when expressed, reduces bile-acid binding by IBABP-L; an siRNA that reduces IBABP-L gene expression; an antisense polynucleotide that reduces IBABP-L gene expression; and a ribozyme that reduces IBABP-L gene expression.

One such composition comprises the expression vector, wherein the sequence encodes a polypeptide selected from the group consisting of: a pro-apoptotic protein; a tumor suppressor; an inhibitor of cell cycle progression; a protein involved in the delivery of toxic secondary bile acids into the cytoplasm of colorectal cancer cells; and an inhibitor of transcriptional activation of IBABP-L gene expression.

Another such composition comprises the expression vector, wherein the sequence encodes a polynucleotide that reduces levels of a polypeptide in the colorectal cancer cell, wherein the polypeptide is selected from the group consisting of IBAB-L, a enzyme of metabolism, a protein essential for cell-cycle progression, a protein that inhibits apoptosis, a protein involved in growth regulatory signal transduction, and a protein involved in bile acid transport out of colorectal cancer cells.

Another such composition comprises the expression vector wherein the sequence encodes a polynucleotide selected from the group consisting of an siRNA, an antisense polynucleotide, and a ribozyme.

Another such composition comprises the expression vector wherein the sequence, when expressed in a colorectal cancer cell that is in the presence of a bile acid, causes apoptosis of the colorectal cancer cell.

Another such composition comprises one or more additional components, including, but not limited to: a carrier; one or more active ingredients that inhibit bile-acid binding activity of IBABP-L, including, but not limited to, a chemotherapeutic drug, a non-steroidal anti-inflammatory drug, a vaccine comprising autologous tumor cells, a vaccine comprising a tumor-associated antigen, a monoclonal antibody directed against a tumor antigen, a recombinant construct for gene correction, a virus-directed enzyme-prodrug treatment, or a matrix metalloproteinase inhibitor.

Such compositions include those that are effective for treating colorectal cancer in a patient in need of such treatment.

According to another embodiment of the invention, methods are provided for treating colorectal cancer in a patient in need of such treatment comprising administering to the patient an effective amount of any of the foregoing compositions.

According to another embodiment of the invention, compositions are provided for treating colorectal cancer in a patient in need of such treatment, such compositions comprising an effective amount of an siRNA construct that reduces levels of IBABP-L polypeptide in the patient. Such compositions include those that reduce levels of IBABP-L polypeptide in the patient without substantially reducing levels of IBABP polypeptide. Such compositions may further comprise a bile acid, including but not limited to cholic acid or deoxycholic acid, and/or a pharmaceutically acceptable carrier. According to a related embodiment of the invention, methods are provided for treating colorectal cancer in a patient in need of such treatment comprising administering to the patient an effective amount of any of the foregoing compositions. Such methods may comprise administering the composition to the patient orally or rectally (for example, by enema).

According to another embodiment of the invention, methods are provided for identifying an agent that is effective in reducing the growth or survival of a colorectal cancer cell comprising: (a) contacting a sample comprising IBABP-L polypeptide with a composition comprising the agent; and (b) determining whether the composition reduces bile-acid binding activity by the IBABP-L polypeptide.

In one such method, the sample is a colorectal cancer cell. Such a method may comprise determining whether the composition reduces levels of IBABP-L polypeptide in the cell. Such a method may also comprise contacting the colorectal cancer cell with the composition and determining whether the composition reduces growth or survival of the colorectal cancer cell.

According to another embodiment of the invention, an inhibitor of IBABP-L activity is used to prepare a medicament to reduce colorectal cancer cell growth or survival.

According to another embodiment of the invention, an inhibitor of IBABP-L activity is used to prepare a medicament to treat colorectal cancer in a male in need of treatment thereof.

The practice of the present invention will employ, unless otherwise indicated, conventional techniques of cell biology, cell culture, molecular biology, microbiology, recombinant DNA, and immunology, which are within the skill of the art. Such techniques are explained fully in the literature. See, for example, Current Protocols in Cell Biology, ed. by Bonifacino, Dasso, Lippincott-Schwartz, Harford, and Yamada, John Wiley and Sons, Inc., New York, 1999; Gene Targeting: A Practical Approach, IRL Press at Oxford University Press, Oxford, 1993; Gene Targeting Protocols, Human Press, Totowa, N.J., 2000; Molecular Cloning A Laboratory Manual, 2nd Ed., ed. by Sambrook, Fritsch and Maniatis (Cold Spring Harbor Laboratory Press: 1989); Culture Of Animal Cells (R. I. Freshney, Alan R Liss, Inc., 1987); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987).

The foregoing and other aspects of the invention will become more apparent from the following detailed description, accompanying drawings, and the claims.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### BRIEF DESCRIPTION OF THE FIGURES

FIG. 1 shows the structure of IBABP-L (fabp6) with exons (E1-E7) and proposed promoters (P1 and P2) labeled (not drawn to scale). P1 drives the expression of IBABP-L, a new variant identified herein (Example 1), which contains seven exons, the first three of which are unique to IBABP-L. P2 promotes the transcription of IBABP, the known form of IBABP, which shares exon 4b to exon 7 with IBABP-L.

FIG. 2 shows the open reading frame of the IBABP gene (SEQ ID NO: 1) (i.e., genomic sequence), which encodes both IBABP-L and IBABP. The open reading frame of IBABP (the 14 kDa form) is underlined, with the additional open reading frame sequence for IBABP-L highlighted in grey. Thus, the open reading frame for IBABP-L contains much of the ORF for IBABP, but also an additional 627 nucleotides on the 5′ end of the gene. The poly(A) signal is bold and underlined.

FIG. 3 shows DNA sequences from the IBABP gene that are unique to IBABP-L (SEQ ID NO: 2) (highlighted in gray in FIG. 2).

FIG. 4 shows an alignment of cDNA sequences for IBABP-L and IBABP. The cDNA sequence for IBABP-L (top line) (SEQ ID NO: 3) is shown with the ATG start site noted in bold. The cDNA sequence for IBABP (bottom line) (SEQ ID NO: 4) are highlighted in gray. Exons 1, 2 and 3 are unique to IBABP-L (note dashes showing a lack of any homologous exon for IBABP). Exon 4a (underlined) is present only in the cDNA for IBABP. Exons 4b-7 are shared by the cDNAs for both IBABP-L and IBABP.

FIG. 5 shows the cDNA sequence encoding IBABP-L (SEQ ID NO: 5).

FIG. 6 shows the nucleotide sequence encoding the N-terminal 49 amino acid sequence from the IBABP-L cDNA (SEQ ID NO: 6).

FIG. 7 shows an alignment of polypeptide sequences for IBABP-L (top line) (SEQ ID NO: 7) and IBABP (bottom line, highlighted in gray) (SEQ ID NO: 8). IBABP-L polypeptide contains a 49 amino acid sequence at its N-terminus that is absent from the IBABP polypeptide.

FIG. 8 shows the predicted polypeptide sequence of IBABP-L (SEQ ID NO: 9). The 49 amino acid N-terminal sequence of IBABP-L that is not found in the IBABP polypeptide is highlighted in gray.

FIG. 9 shows expression of IBABP and IBABP-L in the gastrointestinal tract. RNA extracted from human liver, gallbladder, and sections of the gastrointestinal tract (duodenum through rectum) was used as a template in quantitative RT-PCR aimed at quantifying IBABP and IBABP-L. The expression of each variant was normalized using housekeeping gene ARPP0.

FIG. 10 shows that agonists of FXR and RXR regulate the expression of IBABP but not IBABP-L. Human Caco-2 (enterocyte-like) cells were incubated with FXR agonists chenodeoxycholic acid (CDCA) or deoxycholic acid (DCA) (100 μM) or with the RXR agonist 9cRA (100 nM) for 24 h. The expression of IBABP-L and IBABP was measured by quantitative RT-PCR. The mRNA copy number of each variant is normalized to the expression the housekeeping gene ARPP0. Values in the figure represent the average of three experiments with each replicate performed in duplicate.

FIG. 11 shows up-regulation of IBABP-L in colorectal carcinoma. Total RNA was isolated from 68 sets of matched human colorectal and adjacent normal mucosa and used as template in a two-step quantitative RT-PCR procedure. Variant-specific primers were used to quantify mRNA encoding IBABP-L and IBABP. Values were normalized to expression of ARPP0. The expression difference between carcinoma and normal mucosa was expressed as fold change of IBABP variants between carcinoma and normal mucosa (A) or as the ratio of IBABP-L to IBABP in colorectal carcinoma (R_{C}) versus adjacent normal mucosa (R_{N}) (B). Error bars represent mean±SEM.

FIG. 12 shows the effect of clinical stage on up-regulation of IBABP-L The ratio of change for polyp to normal tissue (R_{P}/R_{N}) and tumor to normal tissue (R_{C}/R_{N}), collectively R_{T}/R_{N} was separated by clinical stage. Bars represent mean±SEM. Difference between R_{P}/R_{N} and R_{C}/R_{N} from Stage II-IV carcinoma is significant (P<0.02).

FIG. 13 shows that a reduction in the expression of IBABP-L with shRNA inhibits the growth of HCT 116 cells. The growth of HCT 116 colorectal cancer cells was monitored over an eight day period. Cells were seeded into the wells of a 96-well microtiter plate at a density of 7000 cells per well. Growth was monitored daily the Promega CellTiter kit. Each day twenty micro liters of this reagent was added to wells and incubated at 37° C. for 1.5 h. The colorimetric reading of wells was recorded at 490 nm. Cell growth is plotted as a percentage of the absorbance of the initial seeding (7000 cells/well). To gauge the effects of knock-down of IBABP-L on growth, cells were transfected with the pSM2c retroviral vector encoding an shRNA targeting IBABP-L (▪). Controls include cells that underwent a mock transfection (♦), cultures of cells transfected with an empty vector (▾), and cells transfected with an irrelevant siRNA (▴).

FIG. 14 shows that IBABP-L is necessary for the survival of colon cancer cells in the presence of secondary bile acids. HCT 116 cells were transfected with pSM2c retroviral vector encoding an shRNA targeting IBABP-L (dark bar), with an irrelevant scrambled shRNA (grey bar), with the empty vector (open bar), or simply subjected to a mock transfection (cross-hatched bar). Cells were seeded at 2,000 cells/well in 96 well plates. At 48 hr, the cells were treated with either vehicle or with 200 μM deoxycholic acid for 24 h. Cell death was monitored using the Cell Death Detection ELISAplus kit (Roche) according to manufacturer's protocol. This kit quantifies DNA fragmentation, a process unique to apoptosis.

FIG. 15 shows the construction of an shRNA vector targeting IBABP-L. A 97 bp double-stranded DNA oligonucleotide (SEQ ID NOS 17 and 18, respectively, in order of appearance) containing a sequence targeting IBABP-L (5′-GCCCGCAACTTCAAGATCGTC-3′ (SEQ ID NO: 19)) and its reverse complemented sequence (5′-GACGATCTTGAAGTTGCGGGC-3′ (SEQ ID NO: 20)) was inserted into retroviral vector pSM2 (Silva et al., Nature Genetics 37:11, 1281-1288, 2005) that can be used to express shRNA in cells. The inserted sequence is transcribed by type III RNA polymerase through U6 promoter, generating a micro RNA. The transcribed microRNA is processed by Drosa and Dicer and turned into a specific siRNA targeting both IBABP-L.

FIG. 16 shows that IBABP-L knockdown increases sensitivity to DCA-induced cell death. (A) HCT116 cells express 13-fold greater levels of IBABP-L mRNA than IBABP mRNA. (B) HCT116 cells are resistant to cell death induced by DCA (100 μM); knockdown of the expression levels of IBABP-L causes the cells to become sensitive to DCA-induced cell death.

FIG. 17 shows the nucleotide sequence (SEQ ID NO: 21) of the IBABP-L promoter from −1563 to +78. The NF-κB binding site (highlighted in gray) starts at −1169 and ends at −1153. The transcription start site (bold and underlined) was determined by primer extension.

FIG. 18 shows deletions of the IBABP-L promoter that reveal the presence of a functional NF-κB binding site in IBABP-L promoter. (FIG. 18 discloses SEQ ID NOS 22-25, 22, 23, 23, 26, 23 and 23, respectively, in order of appearance.)

FIG. 19 shows the results of functional analysis of various deletions of the IBABP-L promoter that show the presence of a functional NF-κB binding site in IBABP-L promoter.

### DETAILED DESCRIPTION OF THE INVENTION

We have identified a new variant of IBABP and designated it as IBABP-L. The transcript for IBABP-L arises from an alternative start site in the IBABP gene and includes three exons that are absent in IBABP. IBABP-L also shares part of a fourth exon with IBABP. The protein encoded by IBABP-L contains a unique 49 amino acid-long N-terminal sequence that is not shared by the IBABP polypeptide. Most significantly, IBABP-L is up-regulated in all stages of colorectal cancer and in malignant colon polyps. By contrast, the expression of the shorter transcript encoding the 14 kDa IBABP is not significantly changed in colorectal cancer.

The IBABP-L transcript is expressed at similar levels throughout the normal human intestine. This is in contrast to the transcript encoding IBABP, which is expressed at levels several orders of magnitude higher in the section of the intestine extending from the jejunum to the ascending colon. In these regions of the intestine, the expression of IBABP-L is at least an order of magnitude lower than IBABP. The two transcripts also differ in their response to bile acids. While bile acids stimulate the expression of IBABP as part of the FXR transcription pathway (Grober et al., J. Biol. Chem. 274:29749-29754, 1999), they are without effect on the expression of IBABP-L.

We compared the expression of IBABP and IBABP-L in colorectal carcinoma samples from 68 patients. IBABP remains essentially unchanged in colorectal cancer, but IBABP-L is up-regulated. In most cases the up-regulation is substantial, with the mean increase in relative mRNA copy number being greater than 30-fold. IBABP-L is up-regulated in early malignant polyps and its high expression is evident in all subsequent clinical classifications of tumor differentiation. Although a trend toward up-regulation in colorectal cancer is evident with PCR primers that fail to distinguish between the two transcripts, a specific measure of IBABP-L is far more sensitive.

IBABP-L is useful as a biomarker. First, the increase in IBABP-L expression in colorectal cancer is independent of the patients' age or gender. Second, based on studies in colon cancer cells lines, the expression of IBABP-L appears to be independent of common oncogenic mutations to proteins like p53, APC, or K-ras. Nevertheless, in conjunction with the fact that IBABP-L is up-regulated in most tumors, the studies from cell lines show that it is highly unlikely that the expression of IBABP-L is dependent on a lesion in a single oncogene. Third, unlike IBABP, the expression of IBABP-L is not influenced by bile acids. Therefore, one would not expect the levels of IBABP-L to be tied to changes in bile acids resulting from dietary changes or overall health status. Collectively, the expression of IBABP-L has many properties that make it well suited for use as a broadly applicable test for colorectal cancer.

We found that the ratio of expression between IBABP-L and IBABP (R_{C}/R_{N}) in samples is a slightly better predictor of colorectal cancer than the relative levels of IBABP-L alone.

We have also found that reducing bile acid binding by IBABP-L through various means can be used to treat colorectal cancer. This can be accomplished by either reducing levels of IBABP-L in a colorectal cancer cell, for example, by expression of siRNA constructs, antisense constructs or ribozymes that reduce IBABP-L expression. Alternatively, this can be accomplished by reducing the bile acid binding activity of IBABP-L, for example by proteins, including but not limited to antibodies, or chemical compounds (i.e., nonproteinaceous compounds that inhibit IBABP-binding activity.

As used herein, the term “colorectal cancer” includes but is not limited to primary colorectal cancer, advanced colorectal cancer, metastatic colorectal cancer. The term “colorectal cancer also includes pre-cancerous conditions characterized by increased expression of IBABP-L as compared with normal, noncancerous cells.

Furthermore, we have found that the IBABP promoter is useful for the expression of various DNA sequences in colorectal cancer cells in order to treat colorectal cancer.

The following definitions and methods are provided to better define the present invention and to guide those of ordinary skill in the art in the practice of the present invention. Unless otherwise noted, terms are to be understood according to conventional usage by those of ordinary skill in the relevant art. Definitions of common terms in molecular biology may also be found in Rieger et al., Glossary of Genetics: Classical and Molecular, 5th edition, Springer-Verlag: New York, 1991; and Lewin, Genes V, Oxford University Press: New York, 1994. The nomenclature for DNA bases as set forth at 37 CFR 1.822 is used. The standard one- and three-letter nomenclature for amino acid residues is used.

### IBABP and Cholesterol Metabolism

Cholesterol is a multifunctional molecule that is essential for a broad array of physiologic processes including membrane biogenesis, caveolae formation, and the distribution of embryonic signaling molecules. It is also as an essential precursor in the synthesis of transcriptionally active lipids including the steroid hormones and oxysterols (Brown and Goldstein, Cell 89:331-340, 1997). Although essential, cholesterol is highly insoluble and can form deposits that contribute to a variety of diseases including gallstones and heart disease (Dowling, Aliment Pharmacol. Ther. 14 Suppl. 2:39-47, 2000; Jones, Am. J. Manag. Care. 7:S289-S298, 2001).

Cholesterol levels are controlled at a variety of levels including intestinal uptake, endogenous biosynthesis, transport, and elimination. The major pathway for cholesterol elimination is via hepatic conversion of cholesterol into water-soluble bile acids (Chiang, Front. Biosci. 3:D176-D193, 1998) and their subsequent secretion into the gastrointestinal tract. Approximately 95% of the secreted bile acids are recycled via intestinal uptake and are returned to the liver through the portal blood. The remaining 5% of bile acids are eliminated from the gut thereby forcing the liver to replenish these losses by converting as much as 0.5 g of cholesterol to bile acids each day (Russell, Cell 97:539-542, 1999). The liver therefore has an enormous capacity to metabolize cholesterol and therapies that target this process have the potential to eliminate cholesterol derived from a variety of sources including diet, synthesis, and atherosclerotic lesions (via the reverse cholesterol transport pathway).

Two metabolic pathways have been identified that convert cholesterol to bile acids (Chiang, Front. Biosci. 3:D176-D193, 1998). In humans, the classic pathway is responsible for at least 90% of all bile acid synthesis. The first and rate-limiting step in this pathway is catalyzed by CYP7A1, a liver-specific cholesterol 7x-hydroxylase. CYP7A1 transcription is strongly repressed by its bile acid end products (Myant et al., J. Lipid Res. 18:135-153, 1977). A member of the nuclear receptor superfamily (FXR, NR1H4, hereafter referred to as BAR) suppresses CYP7A1 transcription in response to endogenous bile acids (Wang et al., Mol. Cell. 3:543-553, 1999; Makishima et al., Science 284:1362-1365, 1999; Parks et al., Science 284:1365-1368, 1999; Sinal et al., Cell 102:731-744, 2000). Two bile acid response elements (BAREs) have been identified in the CYP7A1 promoter. However, BAR is unable to bind directly to either element, suggesting an indirect role for BAR in the regulation of CYP7A 1 (Chiang et al., J. Biol. Chem. 275:10918-10924, 2000). A mechanism has been proposed whereby BAR induces the negative transcriptional regulator SHP (small heterodimer partner), which in turn represses transcription factors that bind to the CYP7A1 BAREs (Lu et al., Mol. Cell. 6:507-515, 2000; Goodwin et al., Mol. Cell. 6:517-526, 2000). This mechanism for CYP7A 1 repression was suggested based on experiments using transiently overexpressed SHP. Because SHP can repress (Lee et al., Mol. Cell. Biol. 20:187-195, 2000; Seol et al., Mol. Endocrinol. 12:1551-1557, 1998) and/or activate (Nishizawa et al., J. Biol. Chem. 277:1586-1592, 2002) numerous nuclear receptors under these conditions, the SHP-induction model does not account for the specificity by which bile acids regulate gene transcription.

Although the mechanisms underlying transrepression by BAR is unclear, it is well known that BAR activates transcription by binding to specific response elements (Forman et al., Cell 81, 687-693, 1995; Laffitte et al., J. Biol. Chem. 275:10638-10647, 2000) as a heterodimer with the nuclear receptor RXR. Several genes have been identified whose transcription is activated by BAR including SHP, the ileal bile acid-binding protein (IBABP), and the hepatic bile salt export pump (BSEP, ABCB11) (Edwards et al., J. Lipid Res. 43:2-12, 2002). These genes are critical for bile acid homeostasis. IBABP is an intracellular protein expressed in the distal ileum where the majority of bile acids are reabsorbed. It has been proposed that IBABP plays a role in transcellular shuttling and/or buffering the high and otherwise toxic levels of bile acids that pass through this tissue. BSEP is a canalicular ATP binding cassette transporter that is responsible for biliary secretion of bile acids. Indeed, inactivating mutations of this gene result in progressive familial intrahepatic cholestosis (type 2) and hepatic cirrhosis (Strautnieks et al., Nat. Genet. 20:233-238, 1998). Thus, in addition to regulating cholesterol degradation, BAR plays a more general role in coordinately regulating bile acid physiology.

BAR also controls other aspects of lipid homeostasis. For example, BAR agonists reduce triglyceride levels (Iser and Sali, Drugs 21:90-119, 1981; Maloney et al., J. Med. Chem. 43, 2971-2974, 2000) and BAR-null mice have elevated triglycerides (Sinal et al., Cell 102:731-744, 2000). This is potentially related to BAR-mediated regulation of apolipoprotein CII and/or phospholipid transfer protein (reviewed in Edwards et al., J. Lipid Res. 43:2-12, 2002). Regardless of the mechanism, it appears that BAR activation promotes reciprocal effects on cholesterol and triglyceride levels. Two classes of BAR modulators have been identified (Dussault et al., J. Biol. Chem. 278:7027-7033, 2003). The first class include agonists that are ˜25-fold more potent than naturally occurring bile acids. These compounds activate BAR and produce the expected regulation pattern on endogenous target genes. AGN34 has been identified as a gene-selective BAR modulator (BARM): it acts as an agonist on CYP7A1, an antagonist on IBABP, and is neutral on SHP (Dussault et al., J. Biol. Chem. 278:7027-7033, 2003).

### Polynucleotides

The transcript (mRNA) for IBABP-L arises from an alternative start site from the start site for the transcript for IBABP. The IBABP-L transcript includes sequences corresponding to three exons (exons 1-3 of the IBABP gene) that are absent from the IBABP transcript and that encode the 49 amino acid sequence at the amino (N) terminus of the IBABP-L polypeptide. Thus, sequences from exons 1-3 are unique to IBABP-L and are useful for producing probes and primers for identifying and quantifying IBABP-L polynucleotides and for other purposes.

As used herein, the term “IBABP-L exon 1-3 polynucleotide (or probe or primer)” refers to a polynucleotide (or probe or primer) that consists of sequences corresponding to exons 1, 2 and 3 from the IBABP gene and that are absent from the IBABP transcript, i.e., the coding region for the 49 amino acid IBABP-L N-terminal polypeptide (as defined below).

As used herein, the term “IBABP-L polynucleotide” refers to this IBABP-L mRNA and the corresponding cDNA, including but not limited to the protein-coding region thereof. Also encompassed by the term “IBABP-L polynucleotides” are, for example: fragments or portions of the IBABP-L mRNA or cDNA, including but not limited to, an IBABP-L exon 1-3 polynucleotide; fragments that encode antigenic determinants of IBABP-L (e.g., those that elicit antibodies that bind selectively to IBABP-L polypeptide); probes and primers that hybridize selectively to IBABP-L polynucleotides; etc. Also included are mutated or variant polynucleotides that include one or more nucleotide insertions, deletions, or substitutions from the wild-type IBABP-L sequence, but that, for example: retain the ability to bind selectively to IBABP-L; encode a polypeptide that includes an IBABP-L antigenic determinant; encode a polypeptide having IBABP-L activity; etc.

As used herein, the term “hybridizes selectively” refers to binding of a probe, primer or other polynucleotide, under stringent hybridization conditions, to a target polynucleotide, such as a native, or wild-type, IBABP-L mRNA or cDNA, to a substantially higher degree than to other polynucleotides. Probes and primers that hybridize selectively to IBABP-L include sequences that are unique to IBABP-L, i.e., exons 1-3. In particular, a probe that “hybridizes selectively” to IBABP-L does not hybridize substantially to IBABP under stringent hybridization conditions and therefore can be used to distinguish an IBABP-L polynucleotide (e.g., an IBABP mRNA) from an IBABP polynucleotide. Similarly, a primer that “hybridizes selectively” to IBABP-L, when used in an amplification reaction such as PCR, results in amplification of IBABP-L without resulting in substantial amplification of IBABP under suitable amplification conditions. Thus, all or substantially all of an IBABP-L-selective probe or primer hybridizes to the target IBABP-L polynucleotide under suitable conditions, as can be determined given the sensitivity of a particular procedure. Similarly, as used herein, the term “selective for” in reference to a polynucleotide, indicates that the polynucleotide hybridizes selectively to a target polynucleotide.

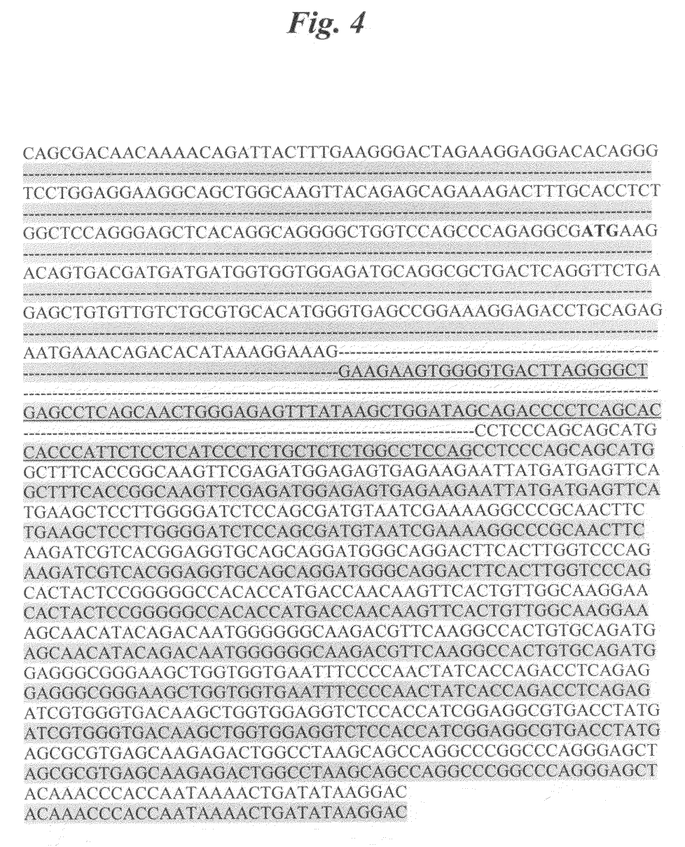

Similarly, a probe or primer that includes a sequence that is unique to IBABP, such as a sequence from exon 4a (see FIG. 4), hybridizes selectively to IBABP. In particular, a probe that hybridizes selectively to IBABP does not hybridize substantially to IBABP under stringent hybridization conditions and therefore can be used to distinguish an IBABP polynucleotide (e.g., an IBABP mRNA) from an IBABP-L polynucleotide. Similarly, a primer that hybridizes selectively to an IBABP polynucleotide, when used in an amplification reaction such as PCR, results in amplification of the IBABP polynucleotide without resulting in substantial amplification of IBABP-L polynucleotide. Thus, all or substantially all of an IBABP-selective probe or primer hybridizes to the target IBABP polynucleotide, as can be determined given the sensitivity of a particular procedure.

As used herein, the term “native IBABP exon 4a polynucleotide (or probe or primer)” refers to a polynucleotide (or probe or primer) that consists of sequences corresponding to exon 4a from the IBABP gene and that are absent from the IBABP-L transcript.

Because sequences from IBABP mRNA are also present in IBABP-L mRNA, a polynucleotide sequence that hybridizes selectively to such shared sequences may also hybridize selectively to a sequence from an IBABP-L polynucleotide. Therefore, a probe or primer that includes sequences of sufficient length that are shared by IBABP and IBABP-L polynucleotides will hybridize under stringent hybridization conditions to both IBABP and IBABP-L, although such sequences do not hybridize to other polynucleotide sequences in a sample under stringent hybridization conditions and thus can be considered to bind “selectively” to IBABP and IBABP-L polynucleotides.

As used herein, the terms “wild-type” or “native” in reference to a polynucleotide are used interchangeably to refer to a polynucleotide that has 100% sequence identity with a reference polynucleotide that can be found in a cell or organism, or a fragment thereof.

Polynucleotide (e.g., DNA or RNA) sequences may be determined by sequencing a polynucleotide molecule using an automated DNA sequencer. A polynucleotide sequence determined by this automated approach can contain some errors. The actual sequence can be confirmed by resequencing the polynucleotide by automated means or by manual sequencing methods well known in the art.

Unless otherwise indicated, each “nucleotide sequence” set forth herein is presented as a sequence of deoxyribonucleotides (abbreviated A, G, C and T). However, the term “nucleotide sequence” of a DNA molecule as used herein refers to a sequence of deoxyribonucleotides, and for an RNA molecule, the corresponding sequence of ribonucleotides (A, G, C and U) where each thymidine deoxynucleotide (T) in the specified deoxynucleotide sequence in is replaced by the ribonucleotide uridine (U).

By “isolated” polynucleotide is intended a polynucleotide that has been removed from its native environment For example, recombinant polynucleotides contained in a vector are considered isolated for the purposes of the present invention. Further examples of isolated polynucleotides include recombinant polynucleotides maintained in heterologous host cells or purified (partially or substantially) polynucleotides in solution. Isolated RNA molecules include in vivo or in vitro RNA transcripts of the DNA molecules of the present invention. Isolated polynucleotides according to the present invention further include such molecules produced synthetically.

Polynucleotides can be in the form of RNA, such as mRNA, or in the form of DNA, including, for instance, cDNA and genomic DNA. The DNA can be double-stranded or single-stranded. A single-stranded DNA or RNA can be a coding strand, also known as the sense strand, or it can be a non-coding strand, also referred to as the anti-sense strand. Polynucleotides can include non-naturally occurring nucleotide or ribonucleotide analogs.

The term “fragment” (of a polynucleotide) as used herein refers to polynucleotides that are part of a longer polynucleotide having a length of at least about 15, 20, 25, 30, 35, or 40 nucleotides (nt) in length, which are useful, for example, as probes and primers. A polynucleotide consisting of a sequence that includes all or part of exons 1-3 of the IBABP-L cDNA (i.e., the sequences that encode the 49 amino acid N-terminal polypeptide of IBABP-L), or a portion thereof, would be considered a fragment of the full-length IBABP-L cDNA, for example. Thus, for example, a fragment of IBABP-L at least 20 nucleotides in length includes 20 or more contiguous bases from the nucleotide sequence of the IBABP-L full-length cDNA. Such DNA fragments may be generated by the use of automated DNA synthesizers or by restriction endonuclease cleavage or shearing (e.g., by sonication) a full-length IBABP-L cDNA, for example.

Also encompassed by the present invention are isolated polynucleotides that hybridize under stringent hybridization conditions to an IBABP-L polynucleotide such as, for example, an IBABP-L transcript (i.e., mRNA). By “stringent hybridization conditions” is intended overnight incubation at 42° C. in a solution comprising: 50% formamide, 5×SSC (750 mM NaCl, 75 mM trisodium citrate), 50 mM sodium phosphate (pH7.6), 5×Denhardt's solution, 10% dextran sulfate, and 20 μg/ml denatured, sheared salmon sperm DNA, followed by washing the filters in 0.1×SSC at about 65° C. Alternatively, stringent hybridizations are conditions used for performance of a polymerase chain reaction (PCR). Such hybridizing polynucleotides are useful diagnostically as a probe according to conventional DNA hybridization techniques or as primers for amplification of a target sequence by the polymerase chain reaction (PCR).

As used herein, the term “hybridizes (or binds) specifically” is used interchangeably with the term “hybridizes (or binds) selectively” means that most or substantially all hybridization of a probe or primer is to a particular polynucleotide in a sample under stringent hybridization conditions.

The present invention also provides polynucleotides that encode all or a portion of a polypeptide, e.g., a full-length IBABP-L polypeptide or a portion thereof. Such protein-coding polynucleotides may include, but are not limited to, those sequences that encode the amino acid sequence of the particular polypeptide or fragment thereof and may also include together with additional, non-coding sequences, including for example, but not limited to introns and non-coding 5′ and 3′ sequences, such as the transcribed, non-translated sequences that play a role in transcription, mRNA processing—including splicing and polyadenylation signals, e.g., ribosome binding and stability of mRNA; an additional coding sequence which codes for additional amino acids, such as those which provide additional functionalities. In addition, the sequence encoding the polypeptide can be fused to a heterogeneous polypeptide or peptide sequence, such as, for example a marker sequence that facilitates purification of the fused polypeptide. One example of such a marker sequence is a hexa-histidine (SEQ ID NO: 41) peptide, such as the tag provided in a pQE vector (Qiagen, Inc.). As described in Gentz et al., Proc. Natl. Acad. Sci. USA 86:821-824 (1989), for instance, hexa-histidine (SEQ ID NO: 41) provides for convenient purification of the fusion protein. The “HA” tag is another peptide useful for purification which corresponds to an epitope derived from the influenza hemagglutinin (HA) protein, which has been described by Wilson et al., Cell 37:767 (1984).

The present invention further relates to variants of the native, or wild-type, polynucleotides of the present invention, which encode portions, analogs or derivatives of an IBABP-L polypeptide. Variants can occur naturally, such as a natural allelic variant, i.e., one of several alternate forms of a gene occupying a given locus on a chromosome of an organism. Non-naturally occurring variants can be produced, e.g., using known mutagenesis techniques or by DNA synthesis. Such variants include those produced by nucleotide substitutions, deletions or additions. The substitutions, deletions or additions can involve one or more nucleotides. The variants can be altered in coding or non-coding regions or both. Alterations in the coding regions can produce conservative or non-conservative amino acid substitutions, deletions or additions. Also included are silent substitutions, additions and deletions, which do not alter the properties and activities of the IBABP-L polypeptide or portions thereof.

Further embodiments of the invention include isolated polynucleotide molecules have, or comprise a sequence having, a high degree of sequence identity with a native, or wild type, IBABP-L polynucleotide, for example, at least 90%, 95%, 96%, 97%, 98% or 99% identical thereto.

A polynucleotide is considered to have a nucleotide sequence at least, for example, 95% “identical” to a reference nucleotide sequence if it is identical to the reference sequence except that it includes up to five mutations (additions, deletions, or substitutions) per each 100 nucleotides of the reference nucleotide sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence. Nucleotide sequence identity may be determined conventionally using known computer programs such as the BESTFIT program (Wisconsin Sequence Analysis Package, Version 8 for Unix, Genetics Computer Group, University Research Park, 575 Science Drive, Madison, Wis. 53711. BESTFIT uses the local homology algorithm of Smith and Waterman, Adv. Appl. Math. 2:482-489 (1981), to find the best segment of homology between two sequences. When using BESTFIT or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed.

### Recombinant Constructs; Vectors and Host Cells

The present invention also provides recombinant polynucleotide constructs that comprise an IBABP-L polynucleotide, including but not limited to vectors. The present invention also provides host cells comprising such vectors and the production of IBABP-L polypeptides or fragments thereof by recombinant or synthetic techniques.

“Operably Linked”. A first nucleic-acid sequence is “operably linked” with a second nucleic-acid sequence when the first nucleic-acid sequence is placed in a functional relationship with the second nucleic-acid sequence. For instance, a promoter is operably linked to a coding sequence if the promoter affects the transcription or expression of the coding sequence. Generally, operably linked DNA sequences are contiguous and, where necessary to join two protein coding regions, in reading frame.

“Recombinant”. A “recombinant” polynucleotide is made by an artificial combination of two otherwise separated segments of sequence, e.g., by chemical synthesis or by the manipulation of isolated segments of polynucleotides by genetic engineering techniques. Techniques for nucleic-acid manipulation are well-known (see, e.g., Sambrook et al., 1989, and Ausubel et al., 1992). Methods for chemical synthesis of polynucleotides are discussed, for example, in Beaucage and Carruthers, Tetra. Letts. 22:1859-1862, 1981, and Matteucci et al., J. Am. Chem. Soc. 103:3185, 1981. Chemical synthesis of polynucleotides can be performed, for example, on commercial automated oligonucleotide synthesizers.

Recombinant vectors are produced by standard recombinant techniques and may be introduced into host cells using well known techniques such as infection, transduction, transfection, transvection, electroporation and transformation. The vector may be, for example, a phage, plasmid, viral or retroviral vector. Retroviral vectors may be replication competent or replication defective. In the latter case, viral propagation generally will occur only in complementing host cells.

Expression vectors include sequences that permit expression of a polypeptide encoded by a polynucleotide of interest in a suitable host cell. Such expression may be constitutive or non-constitutive, e.g., inducible by an environmental factor or a chemical inducer that is specific to a particular cell or tissue type, for example. Expression vectors include chromosomal-, episomal- and virus-derived vectors, e.g., vectors derived from bacterial plasmids, bacteriophage, yeast episomes, yeast chromosomal elements, viruses such as baculoviruses, papova viruses, vaccinia viruses, adenoviruses, fowl pox viruses, pseudorabies viruses and retroviruses, and vectors derived from combinations thereof, such as cosmids and phagemids.

In expression vectors, a polynucleotide insert is operably linked to an appropriate promoter. The promoter may be a homologous promoter, i.e., a promoter or functional portion thereof, that is associated with the polynucleotide insert in nature, for example, an IBABP promoter with an IBABP or IBABP-L protein coding region. Alternatively, the promoter may be a heterologous promoter, i.e., a promoter or functional portion thereof, that is not associated with the polynucleotide insert in nature, for example, a bacterial promoter used for high-level protein expression in bacterial cells (or, for that matter, any promoter other than an IBABP promoter) operably linked to an IBABP-L protein coding region. The expression constructs will further contain sites for transcription initiation, termination and, in the transcribed region, a ribosome binding site for translation. The coding portion of the mature transcripts expressed by the constructs will include a translation initiating AUG at the beginning and a termination codon appropriately positioned at the end of the polypeptide to be translated.

Vectors may include one or more selectable marker suitable for selection of a host cell into which such a vector has been introduced. Such markers include dihydrofolate reductase or neomycin resistance for eukaryotic cell culture and tetracycline or ampicillin resistance genes for culturing in E. coli and other bacteria. Representative examples of appropriate hosts include bacterial cells, such as E. coli, Streptomyces and Salmonella typhimurium cells; fungal cells, such as yeast cells; insect cells such as Drosophila S2 and Spodoptera Sf9 cells; animal cells such as CHO, COS and Bowes melanoma cells; and plant cells. Appropriate culture media and conditions for the above-described host cells are known in the art.

Bacterial promoters suitable include the E. coli lacI and lacZ promoters, the T3 and T7 promoters, the gpt promoter, the lambda PR and PL promoters and the trp promoter. Eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

A polypeptide of interest may be expressed in a modified form, such as a fusion protein, and may include not only secretion signals but also additional heterologous functional regions. For instance, a region of additional amino acids, particularly charged amino acids, may be added to the N-terminus of the polypeptide to improve stability and persistence in the host cell, during purification or during subsequent handling and storage. Also, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art.

An expressed polypeptide of interest can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography.

Polypeptides of the present invention include naturally purified products, products of chemical synthetic procedures, and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes.

### Polypeptides

As used herein, the phrase “an IBABP-L polypeptide” refers to a polypeptide at least 10, 11, 12, 12, 14, 15, 20, 30, 40, 49, 50, 100 or more amino acid residues in length and have a high degree of sequence identity with the full-length native, or wild-type, IBABP-L polypeptide or a fragment thereof. Included are variant forms of IBABP-L polypeptides that include deletions, insertions or substitutions of one or more amino acid residues in a native IBABP polypeptide sequence, including without limitation polypeptides that exhibit activity similar, but not necessarily identical, to an activity of the full-length native, or wild-type, IBABP-L polypeptide or fragment thereof as measured in a relevant biological assay.

As used herein, the terms “wild-type” or “native” in reference to a peptide or polypeptide are used interchangeably to refer to a polypeptide that has 100% sequence identity with a reference polypeptide that can be found in a cell or organism, or a fragment thereof.

As used herein, the term “N-terminal polypeptide of IBABP-L,” or “IBABP-L N-terminal polypeptide,” or simply “N-terminal polypeptide” refers to a unique 49-amino acid sequence at the N-terminus of the IBABP-L polypeptide, which is not part of the IBABP polypeptide.

As used herein, the terms “peptide” and “oligopeptide” are considered synonymous and, as used herein, each term refers to a chain of at least two amino acids coupled by peptidyl linkages. As used herein, the terms “polypeptide” and “protein” are considered synonymous and each term refers to a chain of more than about ten amino acid residues. All oligopeptide and polypeptide formulas or sequences herein are written from left to right and in the direction from amino terminus to carboxy terminus.

As used herein, the term “isolated” polypeptide or protein refers to a polypeptide or protein removed from its native environment. For example, recombinantly produced polypeptides and proteins expressed in host cells are considered isolated for purposes of the invention as are native or recombinant polypeptides and proteins which have been substantially purified by any suitable technique.

As used herein, the term “binds selectively” is interchangeable with the term “binds specifically, and, when used in reference to an IBABP polypeptide, refers to binding of an antibody, ligand, receptor, substrate, or other binding agent to the target IBABP polypeptide to a substantially higher degree than to other polypeptides, such as, for example, to IBABP. According to some embodiments, all or substantially all binding of an antibody or other binding agent is to the target IBABP-L polynucleotide, as can be determined given the sensitivity of a particular procedure. An antibody, ligand, receptor, substrate or other binding agent is said to be “selective for” or specific for” a polypeptide or other target molecule, such as IBABP-L, if it binds selectively to the target molecule.

The amino acid sequence of an IBABP-L polypeptide or peptide can be varied without significant effect on the structure or function of the protein. In general, it is possible to replace residues which contribute to the tertiary structure of the polypeptide or peptide, provided that residues performing a similar function are used. In other instances, the type of residue may be completely unimportant if the alteration occurs at a non-critical region of the protein.

Thus, the invention further includes variations of IBABP-L polypeptide or peptide that show substantial IBABP-L activity. Such mutants include deletions, insertions, inversions, repeats, and type substitutions (for example, substituting one hydrophilic residue for another, but not strongly hydrophilic for strongly hydrophobic as a rule). Small changes or such “neutral” amino acid substitutions will generally have little effect on activity.

Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr.

Guidance concerning which amino acid changes are likely to be phenotypically silent (i.e., are not likely to have a significant deleterious effect on a function) can be found, for example, in Bowie et al., Science 247:1306-1310, 1990.

Thus, a fragment, derivative or analog of a native, or wild-type IBABP-L polypeptide, may be (i) one in which one or more of the amino acid residues are substituted with a conserved or non-conserved amino acid residue and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide, such as an IgG Fc fusion region peptide or leader or secretory sequence or a sequence that is employed for purification of the mature polypeptide or a proprotein sequence.

Charged amino acids may be substituted with another charged amino acid. Charged amino acids may also be substituted with neutral or negatively charged amino acids, resulting in proteins with reduced positive charge. The prevention of aggregation is highly desirable to avoid a loss of activity and increased immunogenicity (Pinckard et al., Clin Exp. Immunol. 2:331-340, 1967; Robbins et al., Diabetes 36:838-845, 1987; Cleland et al., Crit. Rev. Therapeutic Drug Carrier Systems 10:307-377, 1993).

The replacement of amino acids can also change the selectivity of protein binding to cell surface receptors. Ostade et al., Nature 361:266-268 (1993) describes certain mutations resulting in selective binding of TNF-α to only one of the two known types of TNF receptors.

It is well known in the art that one or more amino acids in a native sequence can be substituted with other amino acid(s), the charge and polarity of which are similar to that of the native amino acid, i.e., a conservative amino acid substitution, resulting in a silent change. Conservative substitutes for an amino acid within the native polypeptide sequence can be selected from other members of the class to which the amino acid belongs. Amino acids can be divided into the following four groups: (1) acidic amino acids, (2) basic amino acids, (3) neutral polar amino acids, and (4) neutral, nonpolar amino acids. Representative amino acids within these various groups include, but are not limited to, (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, cystine, tyrosine, asparagine, and glutamine; and (4) neutral nonpolar (hydrophobic) amino acids such as alanine, leucine, isoleucine, valine, proline, phenylalanine, tryptophan, and methionine. Conservative amino acid substitution within the native polypeptide sequence can be made by replacing one amino acid from within one of these groups with another amino acid from within the same group. In one aspect, biologically functional equivalents of the proteins or fragments thereof of the present invention can have ten or fewer, seven or fewer, five or fewer, four or fewer, three or fewer, two, or one conservative amino acid changes. The encoding nucleotide sequence will thus have corresponding base substitutions, permitting it to encode biologically functional equivalent forms of the proteins or fragments of the present invention.

It is understood that certain amino acids may be substituted for other amino acids in a protein structure without appreciable loss of interactive binding capacity with structures such as, for example, antigen-binding regions of antibodies or binding sites on substrate molecules. Because it is the interactive capacity and nature of a protein that defines that protein's biological functional activity, certain amino acid sequence substitutions can be made in a protein sequence and, of course, its underlying DNA coding sequence and, nevertheless, a protein with like properties can still be obtained. It is thus contemplated by the inventors that various changes may be made in the peptide sequences of the proteins or fragments of the present invention, or corresponding DNA sequences that encode said peptides, without appreciable loss of their biological utility or activity. It is understood that codons capable of coding for such amino acid changes are known in the art.

In making such changes, the hydropathic index of amino acids may be considered. The importance of the hydropathic amino acid index in conferring interactive biological function on a protein is generally understood in the art (Kyte and Doolittle, J. Mol. Biol. 157:105-132, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte and Doolittle, J. Mol. Biol. 157:105-132, 1982); these are: isoleucine (+4.5), valine (+4.2), leucine (+3.8), phenylalanine (+2.8), cysteine/cystine (+2.5), methionine (+1.9), alanine (+1.8), glycine (−0.4), threonine (−0.7), serine (−0.8), tryptophan (−0.9), tyrosine (−1.3), proline (−1.6), histidine (−3.2), glutamate (−3.5), glutamine (−3.5), aspartate (−3.5), asparagine (−3.5), lysine (−3.9), and arginine (4.5). In making such changes, the substitution of amino acids whose hydropathic indices may be within ±2, or +1, or within ±0.5.

It is also understood in the art that the substitution of like amino acids can be made effectively on the basis of hydrophilicity. U.S. Pat. No. 4,554,101 states that the greatest local average hydrophilicity of a protein, as govern by the hydrophilicity of its adjacent amino acids, correlates with a biological property of the protein.

As detailed in U.S. Pat. No. 4,554,101, the following hydrophilicity values have been assigned to amino acid residues: arginine (+3.0), lysine (+3.0), aspartate (+3.0.+−0.1), glutamate (+3.0.+−0.1), serine (+0.3), asparagine (+0.2), glutamine (+0.2), glycine (0), threonine (−0.4), proline (−0.5.+−0.1), alanine (−0.5), histidine (−0.5), cysteine (−1.0), methionine (−1.3), valine (−1.5), leucine (−1.8), isoleucine (−1.8), tyrosine (−2.3), phenylalanine (−2.5), and tryptophan (−3.4). In making changes to a native polypeptide or peptide sequence, the substitution of amino acids whose hydrophilicity values may be within ±2, or within ±1, or within ±0.5.

Of course, the number of amino acid substitutions a skilled artisan would make depends on many factors, including those described above. Generally speaking, the number of substitutions for any given IBABP-L polypeptide will not be more than 50, 40, 30, 20, 10, 5, 3, or 2.

Amino acids in the IBABP-L protein of the present invention that are essential for function can be identified by methods known in the art, such as site-directed mutagenesis or alanine-scanning mutagenesis (Cunningham and Wells, Science 244:1081-1085, 1989). The latter procedure introduces single alanine mutations at every residue in the molecule. The resulting mutant molecules are then tested for biological activity such as receptor binding or in vitro, or in vitro proliferative activity. Sites that are critical for ligand-receptor binding can also be determined by structural analysis such as crystallization, nuclear magnetic resonance or photoaffinity labeling (Smith et al., J. Mol. Biol. 224:899-904, 1992; de Vos et al. Science 255:306-312, 1992).

The polypeptides and peptides of the present invention include native, or wild-type polypeptides and peptides, and polypeptides or peptide variants that are at least 75%, 80%, 85%, 90%, 95%, 96%, 97%, 98% or 99% identical to (or have such a degree of identity with) the native IBABP-L polypeptide and fragments thereof.

By a polypeptide having an amino acid sequence at least, for example, 95% “identical” to a reference amino acid sequence is intended that the amino acid sequence of the polypeptide is identical to the reference sequence except that the polypeptide sequence may include up to five amino acid alterations per each 100 amino acids of the reference amino acid sequence of the reference polypeptide. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide has a particular degree of amino acid sequence identity when compared to a reference polypeptide can be determined conventionally using known computer programs such the Bestfit program (Wisconsin Sequence Analysis Package, Version 8 for Unix, Genetics Computer Group, University Research Park, 575 Science Drive, Madison, Wis. 53711. When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

In another embodiment of the present invention, there are provided fragments of the polypeptides described herein. Such fragments include: a polypeptide comprising the 49-amino acid N-terminal sequence of IBABP-L; fragments that include one or more antigenic determinants of IBABP-L, for example, those that elicit antibodies that bind selectively to IBABP-L; and fragments of IBABP-L that bind bile acid. Also included are fragments that include both sequences that are unique to IBABP-L and that are shared by IBABP-L and IBABP. For example, one such fragment is a polypeptide that spans the junction between the 49-amino acid N-terminal sequence of IBABP-L and adjacent sequences in IBABP-L polypeptide (which are also present in IBABP) can be used to raise antibodies that bind specifically to the junction fragment, even if it includes as few as four to six amino acid residues from the N-terminal sequence of IBABP-L. Because such a junction fragment only exists and can be detected if sequences unique to IBABP-L are present, particularly sequences from the 49 amino acid N-terminal polypeptide, antibodies that are elicited by such junction fragments are considered to bind selectively to an IBABP-L polypeptide. The polypeptide fragments of the present invention can be used for numerous purposes, for example, to elicit antibody production in a mammal, as molecular weight markers on SDS-PAGE gels or on molecular sieve gel filtration columns using methods well known to those of skill in the art, etc.

Polypeptides of the present invention can be used to raise polyclonal and monoclonal antibodies, which are useful in diagnostic assays for detecting IBABP-L expression or for other purposes. Further, such polypeptides can be used in the yeast two-hybrid system to “capture” binding proteins (Fields and Song, Nature 340:245-246, 1989).

In another aspect, the invention provides a peptide or polypeptide comprising an epitope-bearing portion of a polypeptide of the invention. The epitope of this polypeptide portion is an immunogenic or antigenic epitope of a polypeptide of the invention. An “immunogenic epitope” is defined as a part of a protein that elicits an antibody response when the whole protein is the immunogen. These immunogenic epitopes are believed to be confined to a few loci on the molecule. On the other hand, a region of a protein molecule to which an antibody can bind is defined as an “antigenic epitope.” The number of immunogenic epitopes of a protein generally is less than the number of antigenic epitopes. See, for instance, Geysen et al., Proc. Natl. Acad. Sci. USA 81:3998-4002, 1984).

As to the selection of peptides or polypeptides bearing an antigenic epitope (i.e., that contain a region of a protein molecule to which an antibody can bind), it is well known in that art that relatively short synthetic peptides that mimic part of a protein sequence are routinely capable of eliciting an antiserum that reacts with the partially mimicked protein. See, for instance, Sutcliffe et al., Science 219:660-666, 1983). Peptides capable of eliciting protein-reactive sera are frequently represented in the primary sequence of a protein, can be characterized by a set of simple chemical rules, and are confined neither to immunodominant regions of intact proteins (i.e., immunogenic epitopes) nor to the amino or carboxyl terminals. Peptides that are extremely hydrophobic and those of six or fewer residues generally are ineffective at inducing antibodies that bind to the mimicked protein; longer, soluble peptides, especially those containing proline residues, usually are effective (Sutcliffe et al., supra, at 661).

Antigenic epitope-bearing peptides and polypeptides of the invention are therefore useful to raise antibodies, including monoclonal antibodies, which bind selectively to a polypeptide of the invention. Thus, a high proportion of hybridomas obtained by fusion of spleen cells from donors immunized with an antigen epitope-bearing peptide generally secrete antibody reactive with the native protein (Sutcliffe et al., supra, at 663). The antibodies raised by antigenic epitope-bearing peptides or polypeptides are useful to detect the mimicked protein, and antibodies to different peptides may be used for tracking the fate of various regions of a protein precursor which undergoes post-translational processing. The peptides and anti-peptide antibodies may be used in a variety of qualitative or quantitative assays for the mimicked protein, for instance in competition assays since it has been shown that even short peptides (e.g., about 9 amino acids) can bind and displace the larger peptides in immunoprecipitation assays. See, for example, Wilson et al., Cell 37:767-778, 1984). The anti-peptide antibodies of the invention also are useful for protein purification, e.g., by adsorption chromatography using known methods.

Antigenic epitope-bearing peptides and polypeptides of the invention designed according to the above guidelines may contain a sequence of at least 7, 8, 9, 10, 11, 12, 13, 14, 15, 20 or 30 or more amino acids contained within the amino acid sequence of a polypeptide of the invention. However, peptides or polypeptides comprising a larger portion of an amino acid sequence of a polypeptide of the invention, containing about 30 to about 50 amino acids, or any length up to and including the entire amino acid sequence of a polypeptide of the invention, also are considered epitope-bearing peptides or polypeptides of the invention and also are useful for inducing antibodies that react with the mimicked protein.

According to one embodiment of the invention, peptides and polypeptides are provided that span the junction between the 49 amino acid N-terminal polypeptide and the remainder of the IBABP-L polypeptide, i.e., that include both unique sequences from IBABP-L (e.g., 4, 5, 6, 7, 8, 9, 10 or more contiguous amino acid residues from the 49 amino acid N-terminal polypeptide of IBABP-L) and sequences that are included in both the IBABP-L and IBABP polypeptides. Such junction-spanning peptides and polypeptides can be used to elicit the production of antibodies in a mammal (e.g., mouse, rat, rabbit, human, etc.) that bind selectively to IBABP-L polypeptide.

The amino acid sequence of the epitope-bearing peptide may be selected to provide substantial solubility in aqueous solvents (i.e., sequences including relatively hydrophilic residues and highly hydrophobic sequences may be avoided).

The epitope-bearing peptides and polypeptides of the invention may be produced by any conventional means for making peptides or polypeptides including recombinant means using nucleic acid molecules of the invention. For instance, a short epitope-bearing amino acid sequence may be fused to a larger polypeptide which acts as a carrier during recombinant production and purification, as well as during immunization to produce anti-peptide antibodies. Epitope-bearing peptides also may be synthesized using known methods of chemical synthesis. For instance, Houghten has described a simple method for synthesis of large numbers of peptides, such as 10-20 mg of 248 different 13 residue peptides representing single amino acid variants of a segment of the HA1 polypeptide which were prepared and characterized (by binding studies employing an enzyme-linked immunosorbent assay [ELISA]) in less than four weeks (Houghten, Proc. Natl. Acad. Sci. USA 82:5131-5135, 1985; and U.S. Pat. No. 4,631,211). In this procedure the individual resins for the solid-phase synthesis of various peptides are contained in separate solvent-permeable packets, enabling the optimal use of the many identical repetitive steps involved in solid-phase methods. A completely manual procedure allows 500-1000 or more syntheses to be conducted simultaneously.

Epitope-bearing peptides and polypeptides of the invention are used to induce antibodies according to methods well known in the art. See, for instance, Sutcliffe et al., supra; Wilson et al., supra; Chow et al., Proc. Natl. Acad. Sci. USA 82:910-914; and Bittle et al., J. Gen. Virol. 66:2347-2354, 1985). Generally, animals may be immunized with free peptide; however, anti-peptide antibody titer may be boosted by coupling of the peptide to a macromolecular carrier, such as keyhole limpet hemacyanin (KLH) or tetanus toxoid. For instance, peptides containing cysteine may be coupled to carrier using a linker such as m-maleimidobenzoyl-N-hydroxysuccinimide ester (MBS), while other peptides may be coupled to carrier using a more general linking agent such as glutaraldehyde. Animals such as rabbits, rats and mice are immunized with either free or carrier-coupled peptides, for instance, by intraperitoneal and/or intradermal injection of emulsions containing about 100 μg peptide or carrier protein and Freund's adjuvant. Several booster injections may be needed, for instance, at intervals of about two weeks, to provide a useful titer of anti-peptide antibody which can be detected, for example, by ELISA assay using free peptide adsorbed to a solid surface. The titer of anti-peptide antibodies in serum from an immunized animal may be increased by selection of anti-peptide antibodies, for instance, by adsorption to the peptide on a solid support and elution of the selected antibodies according to methods well known in the art.

Immunogenic epitope-bearing peptides of the invention, i.e., those parts of a protein that elicit an antibody response when the whole protein is the immunogen, are identified according to methods known in the art. For instance, Geysen et al. (1984), supra, discloses a procedure for rapid concurrent synthesis on solid supports of hundreds of peptides of sufficient purity to react in an enzyme-linked immunosorbent assay. Interaction of synthesized peptides with antibodies is then easily detected without removing them from the support. In this manner a peptide bearing an immunogenic epitope of a desired protein may be identified routinely by one of ordinary skill in the art. For instance, the immunologically important epitope in the coat protein of foot-and-mouth disease virus was located by Geysen et al. with a resolution of seven amino acids by synthesis of an overlapping set of all 208 possible hexapeptides covering the entire 213 amino acid sequence of the protein. Then, a complete replacement set of peptides in which all 20 amino acids were substituted in turn at every position within the epitope were synthesized, and the particular amino acids conferring specificity for the reaction with antibody were determined. Thus, peptide analogs of the epitope-bearing peptides of the invention can be made routinely by this method. U.S. Pat. No. 4,708,781 to Geysen (1987) further describes this method of identifying a peptide bearing an immunogenic epitope of a desired protein.

U.S. Pat. No. 5,194,392 to Geysen (1990) describes a general method of detecting or determining the sequence of monomers (amino acids or other compounds) which is a topological equivalent of the epitope (i.e., a “mimotope”) which is complementary to a particular paratope (antigen binding site) of an antibody of interest. More generally, U.S. Pat. No. 4,433,092 to Geysen (1989) describes a method of detecting or determining a sequence of monomers which is a topographical equivalent of a ligand which is complementary to the ligand binding site of a particular receptor of interest. Similarly, U.S. Pat. No. 5,480,971 discloses linear C₁₋₇-alkyl peralkylated oligopeptides and sets and libraries of such peptides, as well as methods for using such oligopeptide sets and libraries for determining the sequence of a peralkylated oligopeptide that preferentially binds to an acceptor molecule of interest. Thus, non-peptide analogs of the epitope-bearing peptides of the invention also can be made routinely by these methods.

As one of skill in the art will appreciate, polypeptides of the present invention and the epitope-bearing fragments thereof described above can be combined with parts of the constant domain of immunoglobulins (IgG), resulting in chimeric polypeptides. These fusion proteins facilitate purification and show an increased half-life in vivo. This has been shown, e.g., for chimeric proteins consisting of the first two domains of the human CD4-polypeptide and various domains of the constant regions of the heavy or light chains of mammalian immunoglobulins (EPA 394,827; Traunecker et al., Nature 331:84-86 (1988)). Fusion proteins that have a disulfide-linked dimeric structure due to the IgG part can also be more efficient in binding and neutralizing other molecules than the monomeric IBABP-L protein or protein fragment alone (Fountoulakis et al., J. Biochem. 270:3958-3964 (1995)).

### Diagnostic Methods

The present invention provides methods for detecting the presence of IBABP-L polynucleotides (for example, IBABP-L mRNA) or polypeptides in a sample, such as a biological sample from an individual; for quantitating IBABP-L polynucleotides or polypeptides in a sample; for determining an IBABP-L/IBABP polynucleotide or polypeptide ratio in a sample, etc.

In the methods of the present invention, a measurement of IBABP-L polypeptide or polynucleotide or an IBABP-L/IBABP ratio is compared to a “reference.” Depending on the embodiment of the invention, such a reference can include a measurement or ratio in a control sample; a standard value obtained by measurements of a population of individuals; a baseline value determined for the same individual at an earlier timepoint, e.g., before commencing a course of treatment; or any other suitable reference used for similar methods.

As used herein, the term “individual” or “patient” refers to a mammal, including, but not limited to, a mouse, rat, rabbit, cat, dog, monkey, ape, human, or other mammal.

By “biological sample” is intended any biological sample obtained from an individual, including but not limited to, a fecal (stool) sample, body fluid (e.g., blood), cell, tissue, tissue culture, or other source that contains IBABP-L protein or mRNA. Methods for obtaining stool samples, tissue biopsies and other biological samples from mammals are well known in the art.

Detection of mRNA. Total cellular RNA can be isolated from a biological sample using any suitable technique such as the single-step guanidinium-thiocyanate-phenol-chloroform method described in Chomczynski and Sacchi, Anal. Biochem. 162:156-159 (1987). Levels of mRNA encoding IBABP-L are then assayed using any appropriate method. These include Northern blot analysis, S1 nuclease mapping, the polymerase chain reaction (PCR), reverse transcription in combination with the polymerase chain reaction (RT-PCR), and reverse transcription in combination with the ligase chain reaction (RT-LCR).

Northern blot analysis can be performed as described in Harada et al., Cell 63:303-312, 1990). Briefly, total RNA is prepared from a biological sample as described above. For the Northern blot, the RNA is denatured in an appropriate buffer (such as glyoxal/dimethyl sulfoxide/sodium phosphate buffer), subjected to agarose gel electrophoresis, and transferred onto a nitrocellulose filter. After the RNAs have been linked to the filter by a UV linker, the filter is prehybridized in a solution containing formamide, SSC, Denhardt's solution, denatured salmon sperm, SDS, and sodium phosphate buffer. IBABP-L cDNA labeled according to any appropriate method (such as a ³²P-multiprimed DNA labeling system is used as probe. After hybridization overnight, the filter is washed and exposed to x-ray film. cDNA for use as probe according to the present invention is described in the sections above.

S1 mapping can be performed as described in Fujita et al., Cell 49:357-367, 1987). To prepare probe DNA for use in S1 mapping, the sense strand of above-described cDNA is used as a template to synthesize labeled antisense DNA. The antisense DNA can then be digested using an appropriate restriction endonuclease to generate further DNA probes of a desired length. Such antisense probes are useful for visualizing protected bands corresponding to the target mRNA (i.e., mRNA encoding IBABP-L). Northern blot analysis can be performed as described above.

According to one embodiment, levels of mRNA encoding IBABP-L are assayed using a polynucleotide amplification method, including but not limited to a polymerase chain reaction (PCR). One PCR method that is useful in the practice of the present invention is the RT-PCR method described in Makino et al., Technique 2:295-301, 1990), for example. By this method, the radioactivity of the DNA products of the amplification, i.e., the “amplification products” or “amplicons,” in the polyacrylamide gel bands is linearly related to the initial concentration of the target mRNA. Briefly, this method involves adding total RNA isolated from a biological sample in a reaction mixture containing a RT primer and appropriate buffer. After incubating for primer annealing, the mixture can be supplemented with a RT buffer, dNTPs, DTT, RNase inhibitor and reverse transcriptase. After incubation to achieve reverse transcription of the RNA, the RT products are then subject to PCR using labeled primers. Alternatively, rather than labeling the primers, a labeled dNTP can be included in the PCR reaction mixture. PCR amplification can be performed in a DNA thermal cycler according to conventional techniques. After a suitable number of rounds to achieve amplification, the PCR reaction mixture is electrophoresed on a polyacrylamide gel. After drying the gel, the radioactivity of the appropriate bands (corresponding to the mRNA encoding IBABP-L is quantified using an imaging analyzer. RT and PCR reaction ingredients and conditions, reagent and gel concentrations, and labeling methods are well known in the art.

According to one embodiment of an amplification method of the invention, primers are employed that selectively amplify an IBABP-L polynucleotide in a sample, for example, a primer pair including at least one primer that selectively hybridizes to IBABP-L mRNA (e.g., that includes sequences from the region of the IBABP-L mRNA that encodes the IBABP-L N-terminal polypeptide. The second primer can include any sequence from the target IBABP-L polynucleotide, whether such a sequence is unique to IBABP-L or is shared by IBABP-L and IBABP. This embodiment is useful for amplifying only an IBABP-L transcript (mRNA) in a sample, for example.

According to another embodiment of the invention, primers are employed that selectively amplify an IBABP polynucleotide, for example, a primer pair that includes at least one primer that selectively hybridizes to IBABP mRNA (e.g., that includes sequences from exon 4a. The second primer can include any sequence from the target IBABP polynucleotide, whether such a sequence is unique to IBABP-L or is shared by IBABP-L and IBABP. This embodiment is useful for amplifying only an IBABP transcript (mRNA) in a sample, for example.

According to another embodiment of the invention, primers are employed that amplify both an IBABP-L polynucleotide and an IBABP polynucleotide. For example, two primer pairs (i.e., 4 primers) can be used, one pair that selectively amplifies IBABP-L and a second pair that selectively amplifies IBABP, so as to produce amplification products that can be distinguished from one another, for example by length. As one example for illustrative purposes, a four-primer amplification system could include: a primer pair for amplifying IBABP-L mRNA that includes (1) a 5′ primer that includes a sequence from the region of the IBABP-L cDNA that encodes the 49 amino acid N-terminal 49 amino acid N-terminal polypeptide of IBABP-L; and (2) a 3′ primer that includes a sequence from the IBABP-L cDNA that is 3′ to the 5′ primer; and a primer pair for amplifying IBABP mRNA that includes (3) a 5′ primer that includes a sequence from exon 4a (which is unique to the IBABP cDNA); and (4) a 3′ primer that includes a sequence 3′ to exon 4a that is present on IBABP cDNA. Alternatively, a three-primer system can be used, one that hybridizes selectively to IBABP-L, one that hybridizes selectively to IBABP, and a third that hybrids selectively to both IBABP-L and IBABP (i.e., that includes a sequence shared by both IBABP-L and IBABP). As one example for illustrative purposes, a three-primer amplification system could include: (1) a 5′ primer that includes a sequence from the region of the IBABP-L cDNA that encodes the N-terminal 49 amino acid sequence of IBABP-L polypeptide (which is unique to the IBABP-L cDNA); (2) a 5′ primer that includes a sequence from exon 4a (which is unique to the IBABP cDNA); and (3) a 3′ primer that includes a sequence 3′ to exon 4a that is present on both the IBABP-L and IBABP cDNAs. This embodiment is useful, for example, for determining the ration of IBABP-L mRNA to IBABP mRNA in a sample.

The skilled artisan will be able to produce additional primers, primer pairs, and sets of primers for PCR and other amplification methods based on the sequences taught herein.

One embodiment of the present invention is a kit that includes primers useful for amplification methods according to the present invention. Such kits also include suitable packaging, instructions for use, or both.

Another PCR method useful for detecting the presence of and/or quantitating IBABP-L mRNA and protein in a biological sample such as a fecal (e.g., stool) sample, is through the use of “bio-barcode” nanoparticles. For detection and/or quantitation of proteins, for example, two types of capture particles are employed: one is a micro-size magnetic particle bearing an antibody selective for a target protein, and the other is a nanoparticle with attached antibodies selective for the same protein. The nanoparticle also carries a large number (e.g., ˜100) of unique, covalently attached oligonucleotides that are bound by hybridization to complementary oligonucleotides. The latter are the “bio-barcodes” that serve as markers for a selected protein. Because the nanoparticle probe carries many oligonucleotides per bound protein, there is substantial amplification, relative to protein. There is a second amplification of signal in a silver enhancement step. The result is 5-6 orders of magnitude greater sensitivity for proteins than ELISA-based assays, by detecting tens to hundreds of molecules. See, e.g., U.S. Pat. No. 6,974,669. See also, e.g., Stoeva et al., J. Am. Chem. Soc. 128:8378-8379, 2006, for an example of detection of protein cancer markers with bio-barcoded nanoparticle probes. The bio-barcode method can also be used for detecting and/or quantitating mRNA and other polynucleotides in a sample (Huber et al., Nucl. Acids Res. 32:e137, 2004; Cheng et al., Curr. Opin. Chem. Biol. 10:11-19, 2006; Thaxton et al., Clin. Chim. Acta 363:120-126, 2006; U.S. Pat. No. 6,974,669).

Detection and quantitation of IBABP-L polypeptide. Assaying the presence of, or quantitating, IBABP-L polypeptide in a biological sample can occur using any art-known method.

Antibody-based techniques are useful for detecting the presence of and/or quantitating IBABP-L levels in a biological sample. For example, IBABP-L expression in tissues can be studied with classical immunohistological methods. In these, the specific recognition is provided by the primary antibody (polyclonal or monoclonal) but the secondary detection system can utilize fluorescent, enzyme, or other conjugated secondary antibodies. As a result, an immunohistological staining of tissue section for pathological examination is obtained. Tissues can also be extracted, e.g., with urea and neutral detergent, for the liberation of IBABP-L for Western-blot or dot/slot assay (Jalkanen et al., J. Cell. Biol. 101:976-985, 1985; Jalkanen et al., J. Cell. Biol. 105:3087-3096, 1987). In this technique, which is based on the use of cationic solid phases, quantitation of IBABP-L can be accomplished using isolated IBABP-L as a standard. This technique can also be applied to body fluids. With these samples, a molar concentration of IBABP-L will aid to set standard values of IBABP-L content for different tissues, fecal matter, body fluids (serum, plasma, urine, synovial fluid, spinal fluid), etc. The normal appearance of IBABP-L amounts can then be set using values from healthy individuals, which can be compared to those obtained from a test subject.

Other antibody-based methods useful for detecting IBABP-L levels include immunoassays, such as the enzyme linked immunosorbent assay (ELISA), the radioimmunoassay (RIA), and the “bio-barcode” assays described above. For example, IBABP-L-selective monoclonal antibodies can be used both as an immunoabsorbent and as an enzyme-labeled probe to detect and quantify the IBABP-L. The amount of IBABP-L present in the sample can be calculated by reference to the amount present in a standard preparation using a linear regression computer algorithm. Such an ELISA for detecting a tumor antigen is described in Iacobelli et al., Breast Cancer Research and Treatment 11:19-30, 1988. In another ELISA assay, two distinct selective monoclonal antibodies can be used to detect IBABP-L in a body fluid. In this assay, one of the antibodies is used as the immunoabsorbent and the other as the enzyme-labeled probe.

The above techniques may be conducted essentially as a “one-step” or “two-step” assay. The “one-step” assay involves contacting IBABP-L with immobilized antibody and, without washing, contacting the mixture with the labeled antibody. The “two-step” assay involves washing before contacting the mixture with the labeled antibody. Other conventional methods may also be employed as suitable. It is usually desirable to immobilize one component of the assay system on a support, thereby allowing other components of the system to be brought into contact with the component and readily removed from the sample.

Suitable enzyme labels include, for example, those from the oxidase group, which catalyze the production of hydrogen peroxide by reacting with substrate. Glucose oxidase, for example, has good stability and its substrate (glucose) is readily available. Activity of an oxidase label may be assayed by measuring the concentration of hydrogen peroxide formed by the enzyme-labeled antibody/substrate reaction. Besides enzymes, other suitable labels include radioisotopes, such as iodine (¹²⁵I, ¹²¹I), carbon (¹⁴C), sulfur (³⁵S), tritium (³H), indium (¹¹²In), and technetium (⁹⁹Tc), and fluorescent labels, such as fluorescein and rhodamine, and biotin.

In addition to assaying IBABP-L levels in a biological sample obtained from an individual, IBABP-L can also be detected in vivo by imaging. Antibody labels or markers for in vivo imaging of IBABP-L include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma.

An IBABP-L-selective antibody or antibody fragment which has been labeled with an appropriate detectable imaging moiety, such as a radioisotope (for example, ¹³¹I, ¹¹²In, 99 mTc), a radio-opaque substance, or a material detectable by nuclear magnetic resonance, is introduced (for example, parenterally, subcutaneously or intraperitoneally) into the mammal to be examined for a disorder. It will be understood in the art that the size of the subject and the imaging system used will determine the quantity of imaging moieties needed to produce diagnostic images. In the case of a radioisotope moiety, for a human subject, the quantity of radioactivity injected will normally range from about 5 to 20 millicuries of ⁹⁹ mTc. The labeled antibody or antibody fragment will then preferentially accumulate at the location of cells which contain IBABP-L. In vivo tumor imaging is described in Burchiel et al., “Immunopharmacokinetics of Radiolabeled Antibodies and Their Fragments” (Chapter 13 in Tumor Imaging: The Radiochemical Detection of Cancer, Burchiel and Rhodes, eds., Masson Publishing Inc., 1982).

IBABP-L-selective antibodies for use in the present invention can be raised against the intact IBABP-L or an antigenic polypeptide fragment thereof, which may presented together with a carrier protein, such as an albumin, to an animal system (such as rabbit or mouse) or, if it is long enough (at least about 25 amino acids), without a carrier.

As used herein, the term “antibody” (Ab) or “monoclonal antibody” (Mab) is meant to include intact molecules as well as antibody fragments (or “fragment antibodies”) (such as, for example, Fab and F(ab′).sub.2 fragments) which are capable of selectively binding to IBABP-L. Fab and F(ab′).sub.2 fragments lack the Fc portion of intact antibody, clear more rapidly from the circulation, and may have less non-specific tissue binding of an intact antibody (Wahl et al., J. Nucl. Med. 24:316-325, 1983).

The antibodies of the present invention may be prepared by any of a variety of methods. For example, cells expressing the IBABP-L or an antigenic fragment thereof can be administered to an animal in order to induce the production of sera containing polyclonal antibodies. In one method, a preparation of IBABP-L protein is prepared and purified as described above to render it substantially free of natural contaminants. Such a preparation is then introduced into an animal in order to produce polyclonal antisera of greater specific activity.

The antibodies of the present invention include monoclonal antibodies (or IBABP-L binding fragments thereof). Such monoclonal antibodies can be prepared using hybridoma technology (Colligan, Current Protocols in Immunology, Wiley Interscience, New York (1990-1996); Harlow & Lane, Antibodies: A Laboratory Manual, Cold Spring Harbor Press, Cold Spring Harbor, N.Y. (1988), Chapters 6-9, Current Protocols in Molecular Biology, Ausubel, infra, Chapter 11). In general, such procedures involve immunizing an animal (for example, a mouse or rabbit) with an IBABP-L antigen or with an IBABP-L-expressing cell. Suitable cells can be recognized by their capacity to bind anti-IBABP-L antibody. Such cells may be cultured in any suitable tissue culture medium, such as Earle's modified Eagle's medium supplemented with 10% fetal bovine serum (inactivated at about 56° C.), and supplemented with about 10 μg/l of nonessential amino acids, about 1,000 U/ml of penicillin, and about 100 μg/ml of streptomycin. The splenocytes of such mice are extracted and fused with a suitable myeloma cell line. Any suitable myeloma cell line may be employed in accordance with the present invention. After fusion, the resulting hybridoma cells are selectively maintained in HAT medium, and then cloned by limiting dilution as described by Wands et al., Gastroenterology 80:225-232, 1981); Harlow & Lane, infra, Chapter 7. The hybridoma cells obtained through such a selection are then assayed to identify clones which secrete antibodies capable of binding the IBABP-L antigen.

Alternatively, additional antibodies capable of binding to the IBABP-L antigen may be produced in a two-step procedure through the use of anti-idiotypic antibodies. Such a method makes use of the fact that antibodies are themselves antigens, and therefore it is possible to obtain an antibody which binds to a second antibody. In accordance with this method, IBABP-L-selective antibodies are used to immunize an animal, such as a mouse. The splenocytes of such an animal are then used to produce hybridoma cells, and the hybridoma cells are screened to identify clones which produce an antibody whose ability to bind to the IBABP-L-selective antibody can be blocked by the IBABP-L antigen. Such antibodies comprise anti-idiotypic antibodies to the IBABP-L-selective antibody and can be used to immunize an animal to induce formation of further IBABP-L-selective antibodies.

It will be appreciated that Fab and F(ab′)₂ and other fragments of the antibodies of the present invention may be used according to the methods disclosed herein. Such fragments are typically produced by proteolytic cleavage, using enzymes such as papain (to produce Fab fragments) or pepsin (to produce F(ab′)₂ fragments). Alternatively, IBABP-L-binding fragments can be produced through recombinant DNA technology or protein synthesis.

Where in vivo imaging is used to detect enhanced levels of IBABP-L for diagnosis in humans, one may use “humanized” chimeric monoclonal antibodies. Such antibodies can be produced using genetic constructs derived from hybridoma cells producing the monoclonal antibodies described above. Methods for producing chimeric antibodies are known in the art. See, for review, Morrison, Science 229:1202, 1985; Oi et al., BioTechniques 4:214, 1986; Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643, 1984; Neuberger et al., Nature 314:268, 1985.

Further suitable labels for the IBABP-L-selective antibodies of the present invention are provided below. Examples of suitable enzyme labels include malate dehydrogenase, staphylococcal nuclease, delta-5-steroid isomerase, yeast-alcohol dehydrogenase, alpha-glycerol phosphate dehydrogenase, triose phosphate isomerase, peroxidase, alkaline phosphatase, asparaginase, glucose oxidase, beta-galactosidase, ribonuclease, urease, catalase, glucose-6-phosphate dehydrogenase, glucoamylase, and acetylcholine esterase.

Examples of suitable radioisotopic labels include ³H, ¹¹²In, ¹²⁵I, ¹³¹I, ³²P, ³⁵S, ¹⁴C, ⁵¹Cr, ⁵⁷To, ⁵⁸Co, ⁵⁹Fe, ⁷⁵Se, ¹⁵²Eu, ⁹⁰Y, ⁶⁷CU, ²¹⁷C, ²¹⁷At, ²¹²Pb, ⁴⁷SC, ⁰⁹Pd, etc. ¹¹²In has advantages where in vivo imaging is used since its avoids the problem of dehalogenation of the 125I or ¹³¹1-labeled monoclonal antibody by the liver. In addition, this radionucleotide has a more favorable gamma emission energy for imaging (Perkins et al., Eur. J. Nucl. Med. 10:296-301, 1985); Carasquillo et al., J. Nucl. Med. 28:281-287, 1987). For example, In coupled to monoclonal antibodies with 1-(P-isothiocyanatobenzyl)-DPTA has shown little uptake in non-tumorous tissues, particularly the liver, and therefore enhances specificity of tumor localization (Esteban et al., J. Nucl. Med. 28:861-870, 1987).

Examples of suitable non-radioactive isotopic labels include ¹⁵⁷Gd, ⁵⁵Mn, ¹⁶²Dy, ⁵²Tr, and ⁵⁶Fe.

Examples of suitable fluorescent labels include ¹⁵²Eu label, fluorescein, isothiocyanate, rhodamine, phycoerythrin, phycocyanin, allophycocyanin, o-phthaldehyde, and fluorescamine.

Examples of suitable toxin labels include diphtheria toxin, ricin, and cholera toxin. Examples of chemiluminescent labels include luminal, isoluminol, aromatic acridinium ester, imidazole, acridinium salt, oxalate ester, luciferin, luciferase, and aequorin.

Examples of nuclear magnetic resonance contrasting agents include heavy metal nuclei such as Gd, Mn, and Fe.

Typical techniques for binding the above-described labels to antibodies are provided by Kennedy et al. (Clin. Chim. Acta 70:1-31, 1976), and Schurs et al. (Clin. Chim. Acta 81:1-40, 1977). Coupling techniques mentioned in the latter are the glutaraldehyde method, the periodate method, the dimaleimide method, the m-maleimidobenzyl-N-hydroxy-succinimide ester method.

One use for diagnostic compositions and methods of the present invention is for the detection of the presence of colorectal cancer (or another condition marked by the up-regulation and/or increased expression of IBABP-L) in a patient or to identify individuals at increased risk of developing colorectal cancer. Another use is to identify patients who are more likely to be responsive to a therapy, or to monitor the efficacy of therapy, directed toward colorectal cancer. Such therapies include frontline therapy with 5-FU/FA in combination with irinotecan and oxaliplatin or other therapies, including the therapies of the present invention. The diagnostic compositions and method of the present invention are also useful for determining the efficacy of therapeutic agents for treatment and prophylaxis of colorectal cancer, including, but not limited to, agents that inhibit kinases, growth factor inhibitors, NF-κB inhibitors, bile acid replacement therapy, antibody therapy, radiation therapy, and combinations thereof. Various other uses, such as in research and clinical settings will be apparent to the skilled practitioner.

In methods in which is made a measurement of IBABP-L polynucleotide or polypeptide in a sample, or the IBABP-L/IBABP polynucleotide or polypeptide ratio in a sample, the measurement can be compared to a reference, e.g., a similar measurement from a control sample from the individual, a measurement from the individual taken at one or more different timepoints (e.g., a baseline measurement before commencing therapy or a measurement at one or more timepoints during and/or after a course of therapy); a value derived from measurements taken from a population of individuals who are healthy, suffer from various stages of colorectal cancer, are at enhanced risk of developing colorectal cancer, etc.; and other such reference values.

### Therapeutic and Prophylactic Administration of IBABP-L Polypeptide

IBABP-L polypeptides of the present invention may be useful in treating patients at risk for, or suffering from, colorectal cancer or other cancers. As noted in Example 1, IBABP-L may serve as a defense mechanism against secondary bile acid-mediated apoptosis. Increased levels of IBABP-L in the intestinal tract would allow more binding of bile acids, sequestering bile acids extracellularly, decreasing cellular bile acid concentration and thus lessen contact with carcinogens, and providing a protective buffer against bile acid damage. As a result, patients, including but not limited to those having a genetic predisposition toward colorectal cancer or who have been treated for colorectal cancer and for whom recurrence is a threat, may be treated with IBABP-L in order to lessen the likelihood of a colorectal cancer (or its recurrence). Thus, the IBABP-L can be exogenously added to cells, tissues, or the body of an individual to produce a therapeutic effect.

One of ordinary skill will appreciate that effective amounts of a IBABP-L polypeptide can be determined empirically for each condition where administration of a such a polypeptide is indicated. The polypeptide having IBABP-L activity can be administered in pharmaceutical compositions in combination with one or more pharmaceutically acceptable carriers, diluents and/or excipients. As one example for illustrative purposes only, IBABP-L polypeptide can be administered in a capsule or pill having an enteric coating for release in the lower gastrointestinal tract. It will be understood that, when administered to a human patient, the total daily usage of the pharmaceutical compositions of the present invention will be decided by the attending physician within the scope of sound medical judgment. The specific therapeutically effective dose level for any particular patient will depend upon a variety of factors including the type and degree of the response to be achieved; the specific composition an other agent, if any, employed; the age, body weight, general health, sex and diet of the patient; the time of administration, route of administration, and rate of excretion of the composition; the duration of the treatment; drugs (such as a chemotherapeutic agent) used in combination or coincidental with the specific composition; and like factors well known in the medical arts.

The IBABP-L composition to be used in the therapy will also be formulated and dosed in a fashion consistent with good medical practice, taking into account the clinical condition of the individual patient (especially the side effects of treatment with IBABP-L alone), the site of delivery of the IBABP-L composition, the method of administration, the scheduling of administration, and other factors known to practitioners.

An effective amount of a polypeptide, polynucleotide, or other therapeutic substance (or a composition comprising such a therapeutic substance) for purposes herein is thus determined by such considerations. As used herein, “effective amount” refers to an amount of a composition that causes a detectable difference in an observable biological effect, including but not limited to, a statistically significant difference in such an effect. The detectable difference may result from a single substance in the composition, from a combination of substances in the composition, or from the combined effects of administration of more than one composition. For example, an “effective amount” of a composition comprising an IBABP-L polypeptide, a polynucleotide that reduces levels of IBABP-L in a colorectal cancer cell, or other therapeutic substance according to the invention may refer to an amount of the composition that kills a cancer cell, treats or prevents cancer or another disease or disorder, or treats the symptoms of cancer or another disease or disorder, in an individual. A combination of an IBABP-L polypeptide and another substance, e.g., an anti-cancer agent, or other active ingredient, in a given composition or treatment may be a synergistic combination. Synergy, as described for example by Chou and Talalay, Adv. Enzyme Regul. 22:27-55 (1984), occurs when the effect of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased activity, or some other beneficial effect of the combination compared with the individual components.

As used herein, “treating” or “treat” includes (i) preventing or delaying a pathologic condition from occurring (e.g. prophylaxis); (ii) inhibiting the pathologic condition or arresting its development or progression; (iii) relieving the pathologic condition; and/or reducing the severity or duration of one or more symptoms associated with the pathologic condition; or any other clinically relevant measure of efficacy.

The length of treatment needed to observe changes and the interval following treatment for responses to occur appears to vary depending on the desired effect.

One embodiment of a pharmaceutical composition of the invention is a pill or capsule suitable for delivery of IBABP-L polypeptide to the gut of a patient, including but not limited to the colon or rectum.

A therapeutic polypeptide may be administered by sustained-release systems. Suitable examples of sustained-release compositions include semi-permeable polymer matrices in the form of shaped articles, e.g., films, or microcapsules. Sustained-release matrices include polylactides (U.S. Pat. No. 3,773,919, EP 58,481), copolymers of L-glutamic acid and gamma-ethyl-L-glutamate (U. Sidman et al., Biopolymers 22:547-556 (1983)), poly (2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP 133,988). Sustained-release IBABP-L compositions also include a liposomally entrapped IBABP-L polypeptide. Liposomes containing a IBABP-L polypeptide are prepared by methods known per se: DE 3,218,121; Epstein, et al., Proc. Natl. Acad. Sci. USA 82:3688-3692 (1985); Hwang et al., Proc. Natl. Acad. Sci. USA 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal IBABP-L therapy.

For parenteral administration, in one embodiment, the IBABP-L polypeptide is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, according to one embodiment of the invention, the formulation does not include oxidizing agents and other compounds that are known to be deleterious to polypeptides.

The formulations may be prepared by contacting the IBABP-L polypeptide uniformly and intimately with liquid carriers or finely divided solid carriers or both. Then, if necessary, the product is shaped into the desired formulation. If the carrier is a parenteral carrier, or a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

It will be understood that the use of certain of the foregoing excipients, carriers, or stabilizers will result in the formation of IBABP-L salts.

IBABP-L to be used for therapeutic administration must be sterile. Sterility is readily accomplished by filtration through sterile filtration membranes (e.g., 0.2 micron membranes). Therapeutic IBABP-L compositions generally are placed into a container having a sterile access port, for example, an intravenous solution bag or vial having a stopper pierceable by a hypodermic injection needle.

IBABP-L may be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous IBABP-L solution, and the resulting mixture is lyophilized. The infusion solution is prepared by reconstituting the lyophilized IBABP-L using bacteriostatic Water-for-Injection.

### Treatment of Colorectal Cancer by Reducing Intracellular Levels of IBABP-L or by Modulating IBABP-L Activity

Described herein are compositions and methods for inhibiting the growth, or inducing the death, of colorectal cancer cells by reducing intracellular levels of IBABP-L or reducing IBABP-L activity, such as, for example, bile-acid binding by IBABP-L. The methods and compositions of the present invention can be used in relation to any type of colorectal cancer cell, including, for example, primary colorectal cancer cells, advanced colorectal cancer cells, and metastatic colorectal cancer cells, or in pre-cancerous cells of intestine (e.g., the colon or rectum) in which IBABP-L is present at levels that are higher than in comparable non-cancerous cells. As used herein, the term “cell” or “cells” is intended to include not only single cells but also tissues and even whole organisms, as would be appropriate for the context.

“Inhibition of IBABP-L therapy” refers to therapy based on the inhibition of IBABP-L activity. For example, in certain embodiments of the present invention, inhibition of IBABP-L therapy includes inhibiting the activity of IBABP-L polypeptide in a colorectal cancer cell in a patient in need of treatment for colorectal cancer. Inhibition of IBABP-L therapy further includes inhibiting the expression of IBABP-L polypeptide in a colorectal cancer cell. Inhibition of IBABP-L therapy can be applied to any type of colorectal cancer cell, including, for example, primary colorectal cancer cells, advanced colorectal cancer cells, and metastatic colorectal cancer cells, or in pre-cancerous cells of the colon or rectum. In certain other embodiments, the invention relates to agents that inhibit IBABP-L activity, including without limitation, bile acid binding activity.

Inhibition of molecules involved in IBABP-L activation. In certain embodiments, the present invention contemplates inhibiting IBABP-L in colorectal cancer cells by inhibiting one or more of the molecules that are directly or indirectly involved in IBABP-L activation. Molecules involved in IBABP-L activation include molecules, such as NF-κB, a transcription factor that is involved in IBABP-L expression.

Polynucleotides that reduce intracellular levels of IBABP-L. Therapeutic use of molecules that inhibit IBABP-L activity include the delivery to colorectal cancer cells of polynucleotides that reduce intracellular levels of IBABP-L, preferably without substantially affecting levels of IBABP.

In certain embodiments of the invention, IBABP-L activity is inhibited through the use of antisense, ribozyme, RNAi, and other nucleic acid-related methods and compositions for inhibiting an IBABP-L activity. Any of the nucleic acid therapies of the invention may be designed to target a nucleic acid sequence represented in an IBABP-L nucleic acid. In certain embodiments, any of the nucleic acid therapies of the invention may be designed to target a nucleic acid sequence represented in a nucleic acid sequence of a molecule involved in the activation of IBABP-L.

RNA interference. The term “RNA interference” or “RNAi” refers to any method by which expression of a gene or gene product is decreased by introducing into a target cell one or more double-stranded RNAs which are homologous to the gene of interest (particularly to the messenger RNA of the gene of interest). RNAi may also be achieved by introduction of a DNA:RNA hybrid wherein the antisense strand (relative to the target) is RNA. Either strand may include one or more modifications to the base or sugar-phosphate backbone. Any nucleic acid preparation designed to achieve an RNA interference effect is referred to herein as an “siRNA” and includes small interfering RNA (siRNA), short hairpin RNA (shRNA), etc. and mimetics thereof (including but not limited to polynucleotides that include non-canonical nucleoside mimetics such as, for example, 2,4-diflulorotoluoyl ribonucleoside, among others; see, e.g., Xia et al., ACS Chem. Biol. 1:176-183, 2006)

Certain embodiments of the invention make use of materials and methods for effecting a reduction in the expression of one or more IBABP-L genes by means of RNAi. Additional embodiments of the invention make use of materials and methods for effecting knockdown of one or more genes involved in the activation of IBABP-L. RNAi is a process of sequence-specific post-transcriptional gene repression which can occur in eukaryotic cells. RNAi has been shown to be effective in reducing or eliminating the expression of genes in a number of different organisms including Caenorhabditiis elegans (see e.g., Fire et al., Nature 391:806-811, 1998), mouse eggs and embryos (Wianny et al., Nature Cell Biol. 2:70-75, 2000; Svoboda et al., Development 127:4147-4156, 2000), and cultured RAT-1 fibroblasts (Bahramina et al., Mol Cell Biol. 19:274-283, 1999), and appears to be an anciently evolved pathway available in eukaryotic plants and animals (Sharp, Genes Dev. 15:485-490, 2001). RNAi has proven to be an effective means of decreasing gene expression in a variety of cell types including HeLa cells, NIH/3T3 cells, COS cells, 293 cells and BHK-21 cells.

The double stranded oligonucleotides used to effect RNAi may be less than 30 base pairs in length, for example, comprising about 25, 24, 23, 22, 21, 20, 19, 18 or 17 base pairs of ribonucleic acid. Optionally the dsRNA oligonucleotides of the application may include 3′ overhang ends. dsRNAs may be synthesized chemically or produced in vitro or in vivo using appropriate expression vectors. Synthetic RNAs include RNAs that are chemically synthesized using methods known in the art (e.g., Expedite RNA phosphoramidites and thymidine phosphoramidite (Proligo, Germany). Synthetic oligonucleotides may be deprotected and gel-purified using methods known in the art (see e.g., Elbashir et al., Genes Dev. 15:188-200, 2001). Longer RNAs may be transcribed from promoters, such as T7 RNA polymerase promoters, known in the art. A single RNA target, placed in both possible orientations downstream of an in vitro promoter, will transcribe both strands of the target to create a dsRNA oligonucleotide of the desired target sequence. Any of the above RNA species may be designed to include a portion of nucleic acid sequence represented in a IBABP-L nucleic acid. RNAi constructs of the invention further include RNAi constructs designed to include a portion of nucleic acid sequence represented in a gene involved in the activation of IBABP-L. Methods and compositions for designing appropriate oligonucleotides may be found, for example, in U.S. Pat. No. 6,251,588, the contents of which are incorporated herein by reference. Further compositions, methods and applications of RNAi technology are provided in U.S. Pat. Nos. 6,278,039, 5,723,750 and 5,244,805, which are incorporated herein by reference.

A gene is “targeted” by a siNA according to the invention when, for example, the siNA molecule selectively decreases or inhibits the expression of the gene. The phrase “selectively decrease or inhibit” as used herein encompasses siNAs that affects expression of one gene as well those that effect the expression of more than one gene. In cases where an siNA affects expression of more than one gene, the gene that is targeted is effected at least two times, three times, four times, five times, ten times, twenty five times, fifty times, or one hundred times as much as any other gene. Alternatively, a siNA targets a gene when the siNA hybridizes under stringent conditions to the gene transcript. siNAs can be tested either in vitro or in vivo for the ability to target a gene.

A short fragment of the target gene sequence (e.g., 19-40 nucleotides in length) is chosen as the sequence of the siNA of the invention. In one embodiment, the siNA is a siRNA. In such embodiments, the short fragment of target gene sequence is a fragment of the target gene mRNA. In preferred embodiments, the criteria for choosing a sequence fragment from the target gene mRNA to be a candidate siRNA molecule include 1) a sequence from the target gene mRNA that is at least 50-100 nucleotides from the 5′ or 3′ end of the native mRNA molecule, 2) a sequence from the target gene mRNA that has a G/C content of between 30% and 70%, most preferably around 50%, 3) a sequence from the target gene mRNA that does not contain repetitive sequences (e.g., AAA, CCC, GGG, TTT, AAAA, CCCC, GGGG, TTTT), 4) a sequence from the target gene mRNA that is accessible in the mRNA, and 5) a sequence from the target gene mRNA that is unique to the target gene. The sequence fragment from the target gene mRNA may meet one or more of the criteria identified supra. In embodiments where a fragment of the target gene mRNA meets less than all of the criteria identified supra, the native sequence may be altered such that the siRNA conforms with more of the criteria than does the fragment of the target gene mRNA. In preferred embodiments, the siRNA has a G/C/content below 60% and/or lacks repetitive sequences.

In some embodiments, each of the siNAs of the invention targets one gene. In one specific embodiment, the portion of the siNA that is complementary to the target region is perfectly complementary to the target region. In another specific embodiment, the portion of the siNA that is complementary to the target region is not perfectly complementary to the target region. siNA with insertions, deletions, and point mutations relative to the target sequence are also encompassed by the invention. Thus, sequence identity may calculated by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90%, 95%, or 99% sequence identity between the siNA and the portion of the target gene is preferred. Alternatively, the complementarity between the siNA and native RNA molecule may be defined functionally by hybridization. A siNA sequence of the invention is capable of hybridizing with a portion of the target gene transcript under stringent conditions (e.g., 400 mM NaCl, 40 mM PIPES pH 6.4, 1 mM EDTA, 50.degree. C. or 70.degree. C. hybridization for 12-16 hours; followed by washing). A siNA sequence of the invention can also be defined functionally by its ability to decrease or inhibit the expression of a target gene. The ability of a siNA to effect gene expression can be determined empirically either in vivo or in vitro.

In addition to siNAs which specifically target only one gene, degenerate siNA sequences may be used to target homologous regions of multiple genes. WO2005/045037 describes the design of siNA molecules to target such homologous sequences, for example by incorporating non-canonical base pairs, for example mismatches and/or wobble base pairs, that can provide additional target sequences. In instances where mismatches are identified, non-canonical base pairs (for example, mismatches and/or wobble bases) can be used to generate siNA molecules that target more than one gene sequence. In a non-limiting example, non-canonical base pairs such as UU and CC base pairs are used to generate siNA molecules that are capable of targeting sequences for differing targets that share sequence homology. As such, one advantage of using siNAs of the invention is that a single siNA can be designed to include nucleic acid sequence that is complementary to the nucleotide sequence that is conserved between homologous genes. In this approach, a single siNA can be used to inhibit expression of more than one gene instead of using more than one siNA molecule to target different genes.

In some embodiments of the invention, siNA molecules are double stranded. In one embodiment, double stranded siNA molecules comprise blunt ends. In another embodiment, double stranded siNA molecules comprise overhanging nucleotides (e.g., 1-5 nucleotide overhangs, preferably 2 nucleotide overhangs). In a specific embodiment, the overhanging nucleotides are 3′ overhangs. In another specific embodiment, the overhanging nucleotides are 5′ overhangs. Any type of nucleotide can be a part of the overhang. In one embodiment, the overhanging nucleotide or nucleotides are ribonucleic acids. In another embodiment, the overhanging nucleotide or nucleotides are deoxyribonucleic acids. In a preferred embodiment, the overhanging nucleotide or nucleotides are thymidine nucleotides. In another embodiment, the overhanging nucleotide or nucleotides are modified or non-classical nucleotides. The overhanging nucleotide or nucleotides may have non-classical internucleotide bonds (e.g., other than phosphodiester bond).

In embodiments where the siRNA is a dsRNA, an annealing step is necessary if single-stranded RNA molecules are obtained. Briefly, combine 30.mu.l of each RNA oligo 50.mu.M solution in 100 mM potassium acetate, 30 mM HEPES-KOH pH 7.4, 2 mM magnesium acetate. The solution is then incubated for 1 minute at 90.degree. C., centrifuged for 15 seconds, and incubated for 1 hour at 37.degree. C.

In embodiments where the siRNA is a short hairpin RNA (shRNA); the two strands of the siRNA molecule may be connected by a linker region (e.g., a nucleotide linker or a non-nucleotide linker).

Preparation of Polynucleotides Used for RNAi, Antisense, and Ribozyme approaches. Polynucleotides for RNAi, antisense, and ribozyme approaches to reduce intracellular levels of IBABP-L may be prepared by any method known in the art for the synthesis of DNA and RNA molecules. These include techniques for chemically synthesizing oligodeoxyribonucleotides and oligoribonucleotides such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone. The skilled person will be aware of other types of chemical modification which may be incorporated into RNA molecules (see International Publications WO03/070744 and WO2005/045037 for an overview of types of modifications).

In one embodiment, modifications can be used to provide improved resistance to degradation or improved uptake. Examples of such modifications include phosphorothioate internucleotide linkages, 2′-O-methyl ribonucleotides (especially on the sense strand of a double stranded siRNA), 2′-deoxy-fluoro ribonucleotides, 2′-deoxy ribonucleotides, “universal base” nucleotides, 5-C-methyl nucleotides, and inverted deoxyabasic residue incorporation (see generally GB2406568).

In another embodiment, modifications can be used to enhance stability or to increase targeting efficiency. For example, with respect to siRNAs, modifications include chemical cross linking between the two complementary strands of an siRNA, chemical modification of a 3′ or 5′ terminus of a strand of an siRNA, sugar modifications, nucleobase modifications and/or backbone modifications, 2′-fluoro modified ribonucleotides and 2′-deoxy ribonucleotides (see generally International Publication WO2004/029212).

In another embodiment, for example, with respect to siRNAs, modifications can be used to increased or decreased affinity for the complementary nucleotides in the target mRNA and/or in the complementary siRNA strand (see generally International Publication WO2005/044976). For example, an unmodified pyrimidine nucleotide can be substituted for a 2-thio, 5-alkynyl, 5-methyl, or 5-propynyl pyrimidine. Additionally, an unmodified purine can be substituted with a 7-deza, 7-alkyl, or 7-alkenyl purine. In another embodiment, when the siNA is a double-stranded siRNA, the 3′-terminal nucleotide overhanging nucleotides are replaced by deoxyribonucleotides (see generally Elbashir et al., 2001, Genes Dev, 15:188).

Antisense polynucleotides. In further embodiments, the invention relates to the use of isolated “antisense” nucleic acids to inhibit expression, e.g., by inhibiting transcription and/or translation of a IBABP-L nucleic acid. The antisense nucleic acids may bind to the potential drug target by conventional base pair complementarity, or, for example, in the case of binding to DNA duplexes, through specific interactions in the major groove of the double helix. In general, these methods refer to the range of techniques generally employed in the art, and include any methods that rely on specific binding to oligonucleotide sequences.

The antisense oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides or agents facilitating transport across the cell membrane (see, e.g., Letsinger et al., Proc. Natl. Acad. Sci. USA 86:6553-6556, 1989; Lemaitre et al., Proc. Natl. Acad. Sci. USA 84:648-652, 1987; PCT Publication No. WO88/09810, published Dec. 15, 1988) or the blood-brain barrier (see, e.g., PCT Publication No. WO89/10134, published Apr. 25, 1988), hybridization-triggered cleavage agents (see, e.g., Krol et al., BioTechniques 6:958-976, 1988) or intercalating agents. (see, e.g., Zon, Pharm. Res. 5:539-549, 1988). To this end, the oligonucleotide may be conjugated to another molecule, e.g., a peptide, hybridization triggered cross-linking agent, transport agent, hybridization-triggered cleavage agent, etc.

Ribozymes. In certain embodiments, the invention relates to other nucleic acid therapies to inhibit the activity of IBABP-L in colorectal cancer cells, including ribozymes, which are enzymatic RNA molecules capable of catalyzing the specific cleavage of RNA. (For a review, see Rossi, Current Biology 4:469-471, 1994) and DNA enzymes. Ribozyme molecules designed to catalytically cleave IBABP-L MRNA transcripts can also be used to prevent translation of subject IBABP-L mRNAs and/or expression of IBABP-L polypeptides. (See, e.g., PCT International Publication WO90/11364, published Oct. 4, 1990; Sarver et al., Science 247:1222-1225, 1990; and U.S. Pat. No. 5,093,246). DNA enzymes are designed so that they recognize a particular target nucleic acid sequence, much like an antisense oligonucleotide, however much like a ribozyme they are catalytic and specifically cleave the target nucleic acid. Methods of making and administering DNA enzymes can be found, for example, in U.S. Pat. No. 6,110,462.

Small molecule inhibitors of bile acid binding or of IBABP gene expression. Agents contemplated by the invention also include compounds selected from libraries of potential inhibitors of IBABP-L binding of bile acids or of IBABP gene expression. There are a number of different libraries used for the identification of small molecule inhibitors, including: chemical libraries, natural product libraries, and combinatorial libraries comprised of random peptides, oligonucleotides or organic molecules.

A wide variety of chemical libraries may be used. For example, chemical libraries may be used that comprise random chemical structures, some of which are analogs of known compounds or analogs of compounds that have been identified as “hits” or “leads” in other drug discovery screens, some of which are derived from natural products, and some of which arise from non-directed synthetic organic chemistry.

Natural product libraries include collections of products of microorganisms, animals, plants, or marine organisms that are used to create mixtures for screening. Natural product libraries include polyketides, non-ribosomal peptides, and variants (non-naturally occurring) thereof (reviewed in Science 282:63-68 (1998)). Combinatorial libraries include those composed of large numbers of peptides, oligonucleotides, or organic compounds as a mixture. Combinatorial libraries include non-peptide combinatorial libraries. Still other combinatorial libraries include peptide, protein, peptidomimetic, multiparallel synthetic collection, recombinatorial, polypeptide, antibody, and RNAi libraries. For a review of combinatorial chemistry and libraries created therefrom, see Myers, Curr. Opin. Biotechnol. 8:701-707 (1997). Identification of inhibitors through use of the various libraries described herein permits modification of the candidate “hit” or “lead” to optimize the capacity of the “hit” to modulate activity.

Antibodies that modulate bile-acid binding by IBABP-L. Antibodies can be used as modulators of the activity of a particular protein, such as, for example IBABP-L. For example, antibodies can bind to IBABP-L so as to reduce bile-acid binding by IBABP-L, such as, for example, by sterically hindering such binding or altering the conformation of IBABP-L such that bile acid binding is reduced or eliminated. Both monoclonal and polyclonal antibodies (Ab) directed against a particular polypeptide, such as a IBABP-L polypeptide, and antibody fragments such as Fab, F(ab)2, Fv and scFv can be used to block the action of a particular protein, such as IBABP-L.

Variant polypeptides and peptide fragments can agonize or antagonize the function of a particular protein, such as the function of IBABP-L. Examples of such variants and fragments include constitutively active or dominant negative mutants of a particular protein, such as dominant negative mutants of IBABP-L. Antagonistic variants may function in any of a number of ways. One of skill in the art can readily make variants comprising an amino acid sequence at least 60%, 70%, 75%, 80%, 85%, 90%, 95%, 98% or 99% identical to a particular polypeptide, or a fragment thereof, and identify variants that agonize or antagonize the function of IBABP-L. Similarly, one can make peptide mimetics (e.g., peptidomimetics) that agonize or antagonize the function of a IBABP-L polypeptide.

### Pharmaceutical Compositions

Pharmaceutical compositions for use in accordance with the present invention may be formulated in a conventional manner using one or more physiologically acceptable carriers or excipients. Thus, the compounds and their physiologically acceptable salts and solvates may be formulated for administration by, for example, injection, inhalation or insufflation (either through the mouth or the nose) or oral, buccal, parenteral or rectal administration (e.g., by enema).

For example, in certain embodiments, a composition of the invention comprises an RNAi mixed with a delivery system, such as a liposome system, and optionally including an acceptable carrier or excipient.

For such therapy, the compounds of the application can be formulated for a variety of loads of administration, including systemic and topical or localized administration. Techniques and formulations generally may be found in Remington's Pharmaceutical Sciences, Meade Publishing Co., Easton, Pa. Agents of the invention may be administered systemically, including injection intramuscularly, intravenously, intraperitoneally, and subcutaneously. Systemic administration can also be by transmucosal or transdermal means. Transmucosal administration may be through nasal sprays or using suppositories.

Agents of the invention may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

Agents of the invention may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For therapies involving the administration of polynucleotides, such polynucleotides can be formulated for a variety of modes of administration, including systemic and topical or localized administration. For systemic administration, the agents may be injected, including intramuscularly, intravenously, intraperitoneally, intranodally, and subcutaneously for injection. The polynucleotides of the application can be formulated in liquid solutions, preferably in physiologically compatible buffers such as Hank's solution or Ringer's solution. In addition, the polynucleotides may be formulated in solid form and redissolved or suspended immediately prior to use. Lyophilized forms are also included.

Toxicity and therapeutic efficacy of therapeutic agents of the invention can be determined by standard pharmaceutical procedures in cell cultures or experimental animals, e.g., for determining the LD.sub.50 (the dose lethal to 50% of the population) and the ED.sub.50 (the dose therapeutically effective in 50% of the population). The dose ratio between toxic and therapeutic effects is the therapeutic index and it can be expressed as the ratio LD.sub.50/ED.sub.50. Compounds which exhibit large therapeutic indices are preferred. While compounds that exhibit toxic side effects may be used, care should be taken to design a delivery system that targets such compounds to the site of affected tissue, such as the colorectal, in order to minimize potential damage to uninfected cells and, thereby, reduce side effects.

The data obtained from the cell culture assays and animal studies can be used in formulating a range of dosage for use in humans. The dosage of such compounds may lie within a range of circulating concentrations that include the ED.sub.50 with little or no toxicity. The dosage may vary within this range depending upon the dosage form employed and the route of administration utilized. For any compound used in the methods of the invention, the therapeutically effective dose can be estimated initially from cell culture assays. A dose may be formulated in animal models to achieve a circulating plasma concentration range that includes the IC.sub.50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

### Use of the IBABP Promoter to Express Gene Sequences in Colorectal Cancer Cells

According to another embodiment of the invention, therefore, the IBABP promoter is used to drive expression of various proteins in colorectal cancer cells for therapeutic purposes. Such proteins include but are not limited to: pro-apoptotic proteins, such as, for example, cytochrome C, caspases (e.g., caspase 3, 6, 7, 8, 9), and Bax; tumor suppressor proteins such as PTEN, the retinoblastoma protein (pRb); proteins that inhibit cell cycle progression, such as, for example, p21, p27, the APC (adenomatous polyposis coli) protein; proteins involved in the delivery of toxic secondary bile acids into the cytoplasm of colon cancer cells, such as, for example, the apical sodium-dependent bile acid transporter ASBT (SLC10A2); and IBABP-L.

According to another embodiment of the invention, the IBABP promoter is used to drive expression of antisense RNA or siRNA in colorectal cancer cells for therapeutic purposes. For example, the IBABP promoter is useful for driving the expression of antisense RNA or siRNA that reduces the expression of proteins including, but not limited to: enzymes of metabolism like fatty acid synthase, ATP citrate lyase, acetyl co-A carboxylase, or glucose-6 phosphate dehydrogenase; proteins essential for cell cycle progression including cyclin-dependent kinases or skp-2; proteins that inhibit apoptosis including members of the IAP family (c-IAP 1, c-IAP2, XIAP, NAIP, and surviving); proteins involved in growth regulatory signal transduction like the Akt kinase; and proteins involved in bile acid transport out of colon cancer cells, including the Ileocyte Basolateral Organic Solute Transporter (or the organic solute transporter-alpha/beta complex).

### Therapeutic Methods

The present invention encompasses methods for treating, preventing, or managing colon cancer in a patient (e.g., a mammal, especially humans) comprising administering an effective amount of one or more therapeutic agents of the invention.

In one embodiment, a single type of therapeutic agent, e.g., an siRNA, is administered in the therapeutic methods of the invention. In another embodiment, a therapeutic agent of the invention is administered in combination with another therapeutic agent of the invention (e.g., with a second siRNA) and/or with in combination with one or more other standard therapeutic agents that are used for in the treatment, prevention or management of colorectal cancer. The term “in combination with” is not limited to the administration of therapeutic agents at exactly the same time, but rather it is meant that the therapeutic agents of the invention and the other agent are administered to a patient in a sequence and within a time interval such that the benefit of the combination is greater than the benefit if they were administered otherwise. For example, each therapeutic agent may be administered at the same time or sequentially in any order at different points in time; however, if not administered at the same time, they should be administered sufficiently close in time so as to provide the desired therapeutic effect. Each therapeutic agent can be administered separately, in any appropriate form and by any suitable route.

A therapeutically effective amount of a therapeutic agent of the invention provides a therapeutic benefit in the treatment or management of colorectal cancer, for example, an amount that improves overall therapy, reduces or avoids unwanted effects, or enhances the therapeutic efficacy of or synergies with another therapeutic agent. The effective amount of a composition of the invention can be determined by standard research techniques. For example, the dosage of the composition which will be effective in the treatment, prevention or management of the disorder can be determined by administering the composition to an animal model such as, e.g., the animal models disclosed herein or known to those skilled in the art. In addition, in vitro assays may optionally be employed to help identify optimal dosage ranges. Alternatively, the dosage may be determined for an individual by titrating the dose until an effective level is reached.

Selection of the preferred effective amount to be used in dosages can be determined (e.g., via clinical trials) by a skilled artisan based upon the consideration of several factors which will be known to one of ordinary skill in the art. Such factors include the disorder to be treated or prevented, the symptoms involved, the patient's body mass, the patient's immune status and other factors known by the skilled artisan to reflect the accuracy of administered pharmaceutical compositions.

The precise dose to be employed in the formulation will also depend on the route of administration, and the seriousness of the disorder, and should be decided according to the judgment of the practitioner and each patient's circumstances. Effective doses may be extrapolated from dose-response curves derived from in vitro or animal model test systems.

When a therapeutic agent of the invention (e.g. an siRNA) is administered directly to a colorectal cancer tissue, generally an amount of between 0.3 mg/kg-20 mg/kg, 0.5 mg/kg-10 mg/kg, or 0.8 mg/kg-2 mg/kg body weight/day is administered. When the therapeutic agent is administered intravenously, generally an amount of between 0.5 mg-20 mg, or 0.8 mg-10 mg, or 1.0 mg-2.0 mg/injection is administered.

### Formulations and Routes of Administration

The siNAs of the invention may be formulated into pharmaceutical compositions by any of the conventional techniques known in the art (see for example, Alfonso et al., in: The Science and Practice of Pharmacy, Mack Publishing, Easton Pa., 19th ed., 1995). Formulations comprising one or more siNAs for use in the methods of the invention may be in numerous forms, and may depend on the various factors specific for each patient (e.g., the type and severity of disorder, type of siNA administered, age, body weight, response, and the past medical history of the patient), the number and type of siNAs in the formulation, the form of the composition (e.g., in liquid, semi-liquid or solid form), the therapeutic regime (e.g. whether the therapeutic agent is administered over time as a slow infusion, a single bolus, once daily, several times a day or once every few days), and/or the route of administration (e.g., topical, oral, intravenous, intramuscular, intra-arterial, intramedullary, intrathecal, transdermal, subcutaneous, intraperitoneal, intranasal, enteral, or sublingual means).

These compositions can take the form of aqueous and non aqueous solutions, suspensions, emulsions, microemulsions, aqueous and non aqueous gels, creams, tablets, pills, capsules, powders, sustained-release formulations and the like. The siNAs of the invention can also be encapsulated in a delivery agent (including, but not limited to, liposomes, microspheres, microparticles, nanospheres, nanoparticles, biodegradable polymers, hydrogels, cyclodextrins poly(lactic-co-glycolic) acid (PLGA)) or complexed with polyethyleneimine and derivatives thereof (such as polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives).

Pharmaceutical carriers, vehicles, excipients, or diluents may be included in the compositions of the invention including, but not limited to, water, saline solutions, buffered saline solutions, oils (e.g., petroleum, animal, vegetable or synthetic oils), starch, glucose, lactose, sucrose, gelatin, malt, rice, flour, chalk, silica gel, sodium stearate, glycerol monostearate, talc, sodium chloride, dried skim milk, glycerol, propylene, glycol, ethanol, biopolymers (e.g., carbopol, haluronic acid, polyacrylic acid, etc.), dextrose, permeation enhancers (e.g., fatty acids, fatty acid esters, fatty alcohols and amino acids), and hydrophilic polymers (e.g., polycarbophil and polyvinylpyrolidone) and the like. The composition, if desired, can also contain minor amounts of wetting or emulsifying agents, or pH buffering agents.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances, which increase the viscosity of the suspension, such as sodium carboxymethyl cellulose, sorbitol, or dextran. In addition, suspensions of the active compounds may be prepared as appropriate oily injection suspensions. Suitable lipophilic solvents or vehicles include fatty oils such as sesame oil, or synthetic fatty acid esters, such as ethyloleate or triglycerides, or liposomes. Optionally, the suspension may also contain suitable stabilizers or agents which increase the solubility of the compounds to allow for the preparation of highly concentrated solutions.

Pharmaceutical compositions can be administered systemically or locally, e.g., near the intended site of action (i.e., a colorectal cancer tissue). Additionally, systemic administration is meant to encompass administration that can target to a particular area or tissue type of interest.

The therapeutic agents of the present invention can also be formulated in combination with other active ingredients, including but not limited to therapeutic compounds that are used to treat colorectal cancer (e.g., commercially available drugs). Many colorectal cancer patients, such as those whose cancer has spread to the lymph nodes, receive chemotherapy (adjuvant therapy), sometimes in conjunction with radiation therapy, in addition to surgery. Chemotherapy drugs used to treat colorectal cancer include 5′-fluorouracil, leucovorin, irinotecan, and capecitabine. Combinations of such drugs, such as fluorouracil and leucovorin are also used. Other treatments for colorectal cancer and pre-cancerous conditions are also being investigated and may also be employed, including but not limited to: non-steroidal anti-inflammatory drugs (e.g., sulindac and COX-2 inhibitors); immunotherapies such vaccination with autologous tumor cells, vaccination against tumor-associated antigens (such as carcinoembryonic antigen), and monoclonal antibodies directed against tumor antigens (e.g., 17-1A antigen); gene therapy, including gene correction (e.g., to restore the wild-type p53 gene) and virus-directed enzyme-prodrug treatment (e.g., expression of bacterial cytosine deaminase, which converts the antifungal agent fluorocytosine into the antineoplastic agent fluorouracil, or nitroreductase, which converts the prodrug CB1954); matrix metalloproteinase inhibitors, such as marimastat. Any of these treatments, alone or in combination, may be combined with treatment using a therapeutic agent according to the invention, whether by administering a single composition comprising a therapeutic agent according to the invention and one of the treatments for colorectal cancer described above, or by administering a composition comprising a therapeutic agent according to the invention (e.g., an siRNA for reducing levels of IBABP-L in a cell) and a separate composition comprising another treatment for colorectal cancer or other active ingredient.

Alternatively, in the case of polynucleotides such as siRNAs, the therapeutic agent can be expressed directly in cells of interest (e.g., colorectal cancer cells) by transfecting the cells with vectors containing the reverse complement siNA sequence under the control of a promoter. For double stranded polynucleotides such as siNAs, cells can be transfected with one or more vectors expressing the reverse complement siNA sequence for each strand under the control of a promoter. The cell of interest will express the polynucleotide directly without having to be administered a composition of the invention.

The contents of all published articles, books, reference manuals and abstracts cited herein, are hereby incorporated by reference in their entirety to more fully describe the state of the art to which the invention pertains.

The invention will be better understood by reference to the following Examples, which are intended to merely illustrate the best mode now known for practicing the invention. The scope of the invention is not to be considered limited thereto.

### EXAMPLE 1

### IBABP-L as a Biomarker of Colorectal Cancer

We have discovered that IBABP is useful as a biomarker for colorectal cancer. Recent work indicated up-regulation in the expression of IBABP in colorectal tumors (DeGottardi et al., Dig. Dis. Sci. 49:982-989, 2004). However, our our in-depth analysis of the gene structure of IBABP surprisingly reveals a new variant of IBABP that we call IBABP-L. IBABP-L arises from an alternative start site in the IBABP gene and consequently encodes the 49-residue N-terminal sequence of IBABP-L. Most significantly, IBABP-L is up-regulated in all stages of colorectal cancer and in malignant colon polyps. We also show that the up-regulation of IBABP reported in a prior study (DeGottardi et al., Dig. Dis. Sci. 49:982-989, 2004) can be attributed entirely to up-regulation of IBABP-L; the expression of the shorter transcript encoding the 14 kDa IBABP is not significantly changed in colorectal cancer.

### Materials and Methods

Cell lines and tissue samples. Human colorectal cancer cell lines (Caco-2, SW480, HCT116, LS174T, LoVo, SW403, WiDr, and HT-29, obtained from the American Type Culture Collection (Manassas, Va.) were grown in Dulbecco's modified Eagle's media (DMEM; Irvine Scientific, Santa Ana, Calif.) containing 1 mM sodium pyruvate, 4.5 g/L D-glucose, 4 mM L-glutamine, and supplemented with 10% fetal bovine serum (Irvine Scientific), and 100 U/ml penicillin, 100 μg/ml streptomycin and 0.25 μg/ml amphotericin B (Omega Scientific, Tarzana, Calif.). Cells were maintained in 100 mm standard cell culture dishes (Falcon, BD Biosciences, San Jose, Calif.) and grown at 37° C. under 5% CO₂.

Matched human colorectal carcinoma and adjacent normal mucosa were purchased from Asterand, Inc. (Detroit, Mich.), or obtained from the Cooperative Human Tissue Network (service of the National Cancer Institute, Bethesda, Md.); patients have provided written consent for use of tissues for scientific purpose. In total, 68 matched human colorectal carcinoma samples were examined (52.9% male donor, 86.8% Caucasian). Patient ages range from 21 to 89 years (76.5% greater than 50 years old). Colorectal carcinomas were distributed by intestinal region as follows: cecum, 11.3%; colon, 48.3%; and rectum, 40.3%; by histological typing: well-differentiated, 27.1%; moderately differentiated, 61.0%; and poorly differentiated, 11.9%; and by clinical stage: stage I, II; stage II, 20; stage III, 17; stage IV, 15; unclassifiable, 5 (not specified in pathology reports provided). Eleven polyp samples were also examined, five of which contained focal high grade dysplasia, one case from the familial adenomatous polyposis (FAP) family, and one case of inherited juvenile polyposis syndrome.

Assessing the expression of IBABP by PCR. Expression of mRNA encoding IBABP-L (Genbank accession number DQ132786) and IBABP (Genbank accession number NM_{—}001445) along the digestive tract was measured with RNA from normal human intestine and liver purchased from Invitrogen and from Biochain Institute, Inc. (Hayward, Calif.) by quantitative RT-PCR. Expression of ARPP0 was used as a control. The expression of IBABP and IBABP-L in human tumor and adjacent normal tissue was measured with tissues purchased from Asterand, Inc. (Detroit, Mich.), and the Cooperative Human Tissue Network as described above.

Total RNA was isolated from tissues using TRIZOL reagent (Invitrogen, Carlsbad, Calif.) in a protocol combined with the RNeasy Mini Kit (Qiagen Inc., Valencia, Calif.). For each sample, frozen tissue (approximately 0.1 g) was cut and soaked in pre-chilled RNA later-ICE stabilizing solution (1.0 ml; Ambion Inc., Austin, Tex.) for 24 h at −20° C. Tissue was minced using a surgical scalpel, immersed in TRIZOL (1.0 ml), and homogenized using a Tissue-Tearor (BioSpec Products, Inc., Bartlesville, Okla.). Chloroform (200 μl) was added to homogenized tissue and sample was mixed by vortexing for 30 s. Samples were centrifuged (12,000×g, 10 minutes at 4° C.) to separate phases. The aqueous phase was removed, added to an equal volume of 70% ethanol, mixed by pipetting, and loaded into the RNeasy column. Following RNA binding, an on-column DNase digestion protocol using RNase-Free DNase Set (Qiagen) was performed according to manufacturer instructions.

To determine the relative expression levels of IBABP-L and IBABP, a two-step quantitative RT-PCR procedure was used. In the first step, complementary DNA (cDNA) was synthesized from total RNA. For each sample, RNA (2.0 μg) was reverse transcribed using SuperScript First-Strand Synthesis System for RT-PCR (Invitrogen) in a 20 μl final reaction volume containing: 10 mM dNTP mix (1.0 μl), 0.5 ug/ml Oligo(dT)₁₂₋₁₈ (1.0 μl), 0.1 M DTT (2.0 μl), 25 mM MgCl2 (4.0 μl), 10×RT buffer (2.0 μl), RNaseOUT Recombinant RNase Inhibitor (1.0 μl), and SuperScript II Reverse Transcriptase (1.0 μl). Reverse transcription was performed at 42° C. for 50 min and terminated by heating to 70° C. for 15 minutes followed by chilling samples on ice. Template RNA was cleaved by incubating with RNase H (1.0 μl) for 20 min at 37° C. In the second step, quantitative PCR (QPCR) was carried out on a Mx 3000P Real-Time PCR System (Stratagene, La Jolla, Calif.) using a solution containing diluted cDNA (1:20; 2.0 μl), 1×SYBR Green PCR Master Mix (Applied Biosystems, Foster City, Calif.), and primers for IBABP-L, IBABP, or ARPP0 (0.25 μM). The primer sets are IBABP: 5′ CCACCCATTCTCCTCATCCCTCTGCTC 3′ (SEQ ID NO: 10) (in exon 4a), 5′ ACCAAGTGAAGTCCTGCCCATCCTG 3′ (SEQ ID NO: 11) (in exon 5); IBABP-L: 5′ ACATGGGTGAGCCGGAAAGGAGAC 3′ (SEQ ID NO: 12) (in exon 3), 5′ CCGGAGTAGTGCTGGGACCAAGTGAAGT 3′ (SEQ ID NO: 13) (in exon 5); ARPP0: 5′CAAGACTGGAGACAAAGTGG 3′ (SEQ ID NO: 14), 5′ AATCTGCAGACAGACACTGG 3′ (SEQ ID NO: 15)). All primers were designed using PrimerSelect™ (DNASTAR, Inc., Madison, Wis.) and synthesized by Integrated DNA Technologies, Inc. (Coralville, Iowa). The following cycling parameters were used: denaturation at 95° C. for 15 s, annealing at 56° C. for 20 s, extension at 72° C. for 30 s, and detection at 78° C. for 5 s. After 40 cycles, PCR products were subjected to dissociation curve analysis to check the PCR specificity. Values obtained from QPCR were normalized to expression of ARPP0.

Regulation of expression of the IBABP variants in Caco-2 cells. Caco-2 cells were seeded in six-well plates (Falcon) at 2×10⁵ cells/well. Medium was exchanged every two days until cells reached 100% confluence and began spontaneous differentiation. Stock solutions of CDCA and DCA (Sigma-Aldrich Co., St. Louis, Mo.), as free acids, were prepared in absolute ethanol (100 mM) and stored at −20° C. A stock solution of 9-cis-retinoic acid (9cRA; Sigma-Aldrich) was dissolved in dimethyl sulfoxide (DMSO; 100 μM) and stored at −20° C. Confluent Caco-2 cells were incubated at 37° C. for 24 h in medium containing CDCA or DCA (100 μM), 9cRA (100 nM), or DMEM only (0.1% solvent concentration). Cells were harvested, cellular RNA was isolated using the RNeasy Mini Kit (Qiagen) according to manufacturer instruction, and expression of IBABP variants was determined by QPCR as described above.

Determination of the transcription start site. Primer extension was conducted using Human Small Intestine Marathon-Ready cDNA with Advantage 2 PCR kit (Clontech) according to the manufacture's manual. Briefly, first PCR was performed using primers AP1 (included in the kit) and gene specific primer 1 (GSP1) 5′-ACATTATATTTTCTTGCCAAGTAGAGGA-3′ (SEQ ID NO: 27) (exon 2), followed by a nested PCR with primer AP2 (included in the kit) and GSP2 5′-ACTTGCCAGCTGCCTTCCT-3′ (SEQ ID NO: 28) (exon 3). The longest PCR product was cloned into pCR2.1 T/A cloning vector (Invitrogen) and 12 individual clones were sequenced. The transcription start site was unanimously located 78 nucleotides away from GSP2. To simplify the description, the position of the TSS “C” was set to +1.

Generation of antibodies to IBABP-L and immunohistochemical studies. A peptide with sequence CTWVSRKGDLQRMKQTHKGKPPSS (SEQ ID NO: 16), which is present in the 49-residue N-terminal sequence of IBABP-L, was synthesized, conjugated to keyhole limpet hemocyanin (KLH), and used as an antigen to immunize rabbits. The antisera from immunized animals were tested for reactivity against recombinant IBABP-L and IBABP by Western blot. Recombinant IBABP-L and IBABP were expressed and purified using pGEX system. Recombinant proteins were expressed as fusion proteins with a Histidine-Tag, which was removed by digestion with thrombin while the protein was bound to a nickel resin. After digestion thrombin was removed with benzamidine-Sepharose.

Paraffin-embedded slides were stained with anti IBABP-L antiserum (1:2000) and followed by a diaminobenzidine-based detection method employing horseradish peroxidase system. The slides were then counterstained with hematoxylin.

Data analysis and statistics. The change in RNA expression of IBABP-L and IBABP between diseased colon tissue and matched adjacent normal mucosa was analyzed using two strategies. In the first, expression level of the variant in cancer or polyp tissue to that of its adjacent normal mucosa resulted in a fold of change value for each variant. Using a two-fold cut-off limit, a value of 2.0 or greater denoted up-regulation, whereas a value of less than 0.5 denoted down-regulation. In the second method, we calculated the ratio of IBABP-L to IBABP in cancer or polyp tissue (R_{C} and R_{P}) and in adjacent normal mucosa (R_{N}). Again using a two-fold cut-off, a ratio greater than or equal to 2.0 denoted up-regulation of IBABP-L in cancer or polyp; a value of less than 0.5 denotes down-regulation. The value from both strategies was grouped according to the parameters of clinical specimen (gender, age, race, tumor size, tumor locale, differentiation level, and clinical stage) and correlation between expression level and clinical parameters was analyzed by t-test and one way ANOVA.

### Results

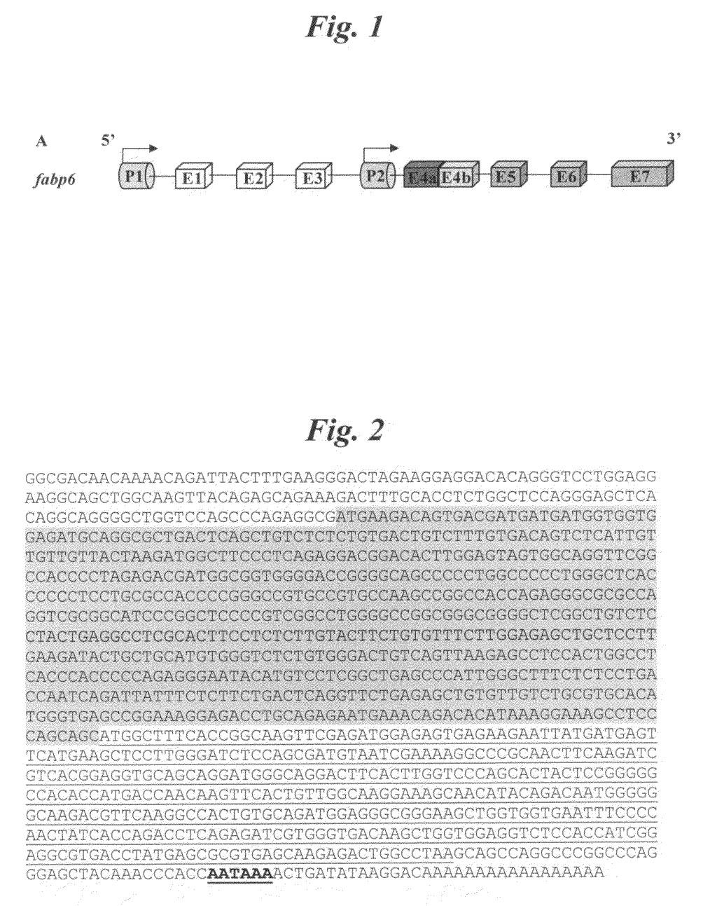

Characterization of the gene encoding IBABP-L. A BLASTN search of the NCBI human expression sequence tag (EST) database (http://www.ncbi.nlm.nih.gov/) using NM_{—}001445 revealed two ESTs (BM974219 and BU683560) that was largely identical to IBABP, except that the newly discovered transcript encoded a protein having a 49-aa sequence at its N-terminus that is not present in IBABP. Since the transcript of this variant is longer than that of IBABP, we call it IBABP-L. The gene for IBABP-L (chromosome 5q33.3-q34; contig NT_{—}023133) is identical to that of the known form, IBABP. However, the mRNA encoding IBABP-L contains seven exons, three of which are unique and are present at the 5′ end of the gene. The shorter protein IBABP contains only four exons and its transcription is initiated within the third intron of the IBABP-L (fabp6) gene. FIG. 1 shows the structure of IBABP-L (also called fabp6).

Thus, the two variants of IBABP share exons 5 through 7. Transcripts encoding both variants are detected in human intestine. The presence of exons unique to IBABP-L permitted the design of variant-specific primers to distinguish expression of IBABP-L from IBABP. As described below, these primers were used to detect the expression of each variant in mRNA extracted from normal human intestine by RT-PCR.

The complete nucleotide sequence of the IBABP-L transcript was deposited in Genebank with accession number DQ132786.

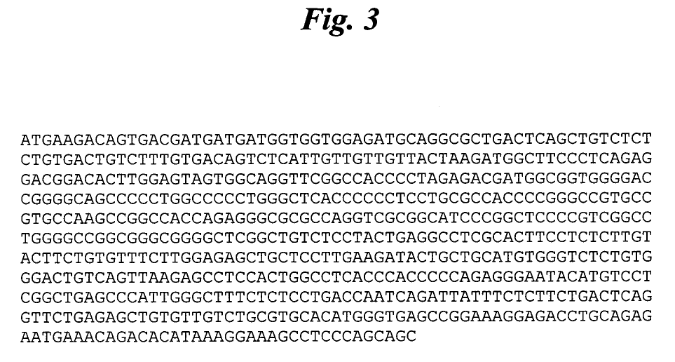

FIG. 2 shows the open reading frame of the IBABP gene (i.e., genomic sequence), which encodes both IBABP-L and IBABP. In FIG. 2, the open reading frame of IBABP (the 14 kDa form) is underlined, with the additional open reading frame sequence for IBABP-L highlighted (grey). Thus, the open reading frame for IBABP-L contains much of the ORF for IBABP, but also an additional 627 nucleotides on the 5′ end of the gene. FIG. 3 shows DNA sequences from the IBABP gene that are unique to IBABP-L (highlighted in gray in FIG. 2).

FIG. 4 shows an alignment of cDNA sequences for IBABP-L and IBABP. The cDNA sequence for IBABP-L (top line) is shown with the ATG start site noted in bold. The cDNA sequence for IBABP (bottom line) are highlighted in gray. Exons 1, 2 and 3 are unique to IBABP-L (note dashes showing a lack of any homologous exon for IBABP). Exon 4a (underlined) is present only in the cDNA for IBABP. Exons 4b-7 are shared by the cDNAs for both IBABP-L and IBABP.

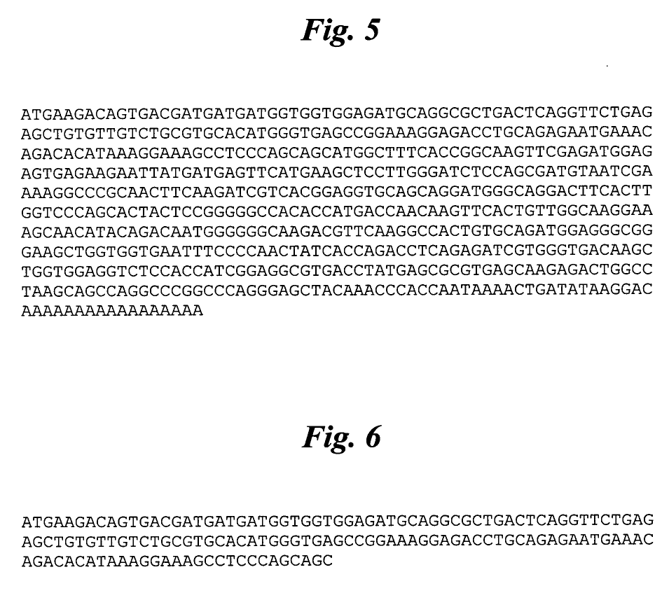

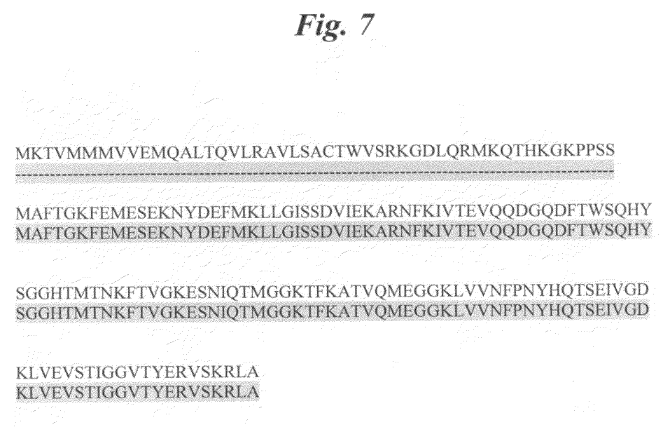

FIG. 5 shows the cDNA sequence encoding IBABP-L, and FIG. 6 shows the nucleotide sequence encoding the N-terminal 49 amino acid sequence from the IBABP-L cDNA.

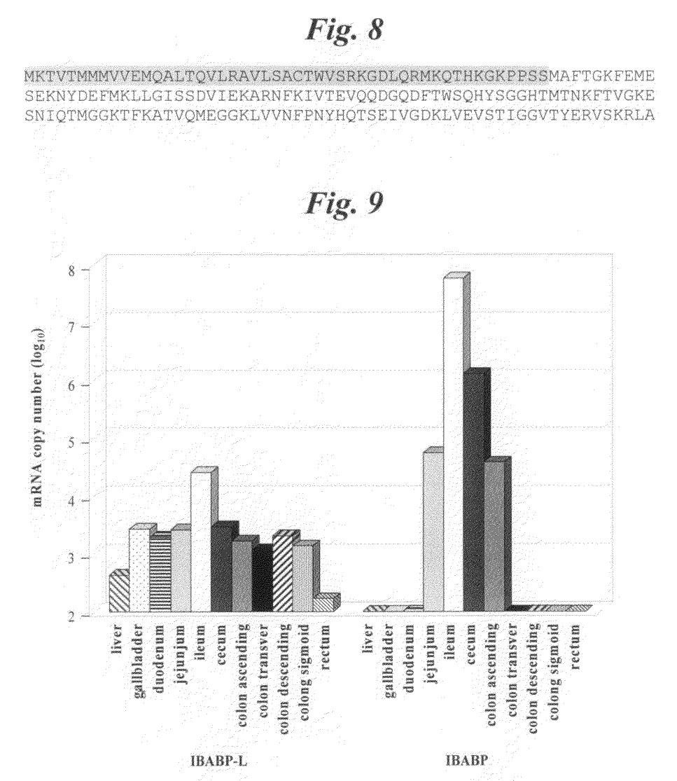

FIG. 7 shows an alignment of polypeptide sequences for IBABP-L (top line) and IBABP (bottom line, highlighted in gray). IBABP-L polypeptide contains a 49 amino acid sequence at its N-terminus that is absent from the IBABP polypeptide. FIG. 8 shows the predicted polypeptide sequence of IBABP-L. The 49 amino acid N-terminal sequence of IBABP-L that is not found in the IBABP polypeptide is highlighted in gray.

Expression pattern of IBABP and IBABP-L in gastrointestinal tissue. The IBABP gene is primarily expressed in the intestine. Therefore, we compared the expression of the transcripts encoding IBABP and IBABP-L in the gastrointestinal tract, particularly tissues associated with the enterohepatic bile acid cycle (human liver, gallbladder and intestinal sections). Oligonucleotides capable of selectively priming the amplification of each variant were used to initiate real-time Q-PCR reactions. The copy number of mRNA transcripts was normalized to the expression of the housekeeping gene acidic ribosomal phosphoprotein (ARPP0), also known as ribosomal protein large P0 (RPLP0), often used as an endogenous control in prostate and colon cancer research (Chene et al., Int. J. Cancer 111:798-804, 2004; Cacev et al., Gut 54:1129-1135, 2005). FIG. 9 shows expression of IBABP and IBABP-L in the gastrointestinal tract. The transcript encoding IBABP-L was found at similar levels in all tissues tested with the exception of the rectum where it was expressed at lower levels (FIG. 9). By contrast, the expression of IBABP is localized to a section of the intestine extending from the jejunum through ascending colon. In these sections, the expression of IBABP was ten to one thousand-fold higher than the expression of IBABP-L.

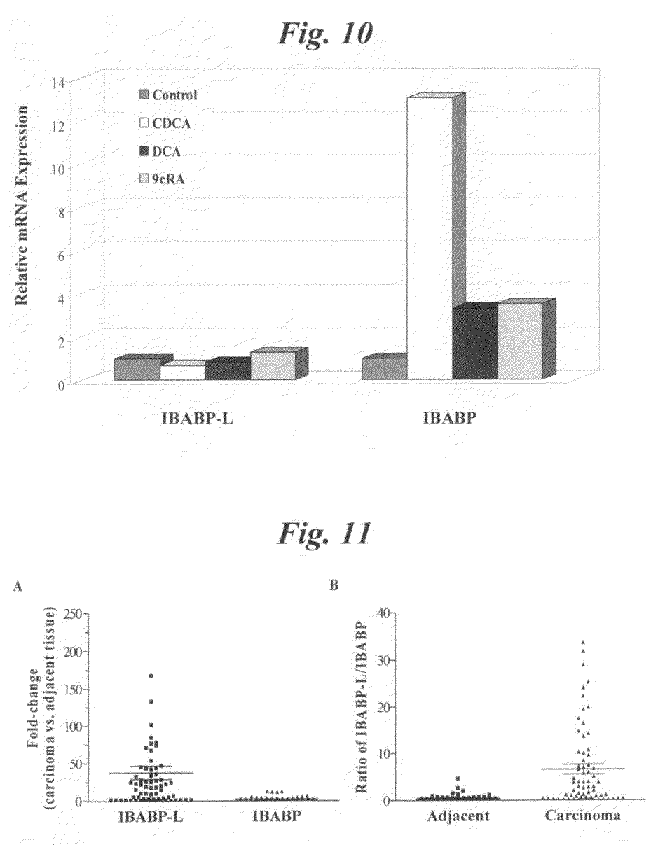

Bile acids differentially regulate the variants of IBABP. Many of the genes involved in bile acid homeostasis are regulated through the FXR nuclear hormone receptor (Forman et al., Cell 81:687-693, 1995), which binds directly to bile acids (Makishima et al., Science 284:1362-1365, 1999; Parks et al., Science 284:1365-1368, 1999). In fact, the expression of IBABP is also regulated by the FXR (Kanda et al., Biochem. J. 330 (Pt. 1):261-265, 1998; Grober et al., J. Biol. Chem. 274:29749-29754, 1999). The effect of bile acids and 9-cis-retinoic acid, a ligand for RXR (the partner of FXR) on the expression of IBABP-L and IBABP was studied. Caco-2 cells were treated with either chenodeoxycholic acid (CDCA), deoxycholic acid (DCA), or 9cRA, and the relative expression of transcripts encoding IBABP-L and IBABP was measured by quantitative RT-PCR (FIG. 10). As expected, CDCA and DCA increased expression of IBABP up to 13-fold, but unexpectedly these agents were without effect on expression of IBABP-L. Similarly, 9cRA also elicited the up-regulated of IBABP, but was without effect on IBABP-L. These results are consistent with the idea that the two variants of IBABP arise from separate transcription start sites.

mRNA encoding IBABP-L is up-regulated in colorectal cancer. The expression of each variant of IBABP was measured in various stages of colon cancer by quantitative RT-PCR using IBABP or IBABP-L selective primer sets. In all cases the level of the transcript in carcinoma was compared to its levels in adjacent normal tissue. The transcript encoding IBABP-L was substantially up-regulated in colon carcinoma; in some cases it was expressed at levels more than 100-fold higher than in normal tissue (FIG. 11A). IBABP-L was up-regulated in 76% (52/68) of colorectal cancers using two-fold cut-off (P<0.0001). In contrast, there was no significant change in the expression of IBABP in carcinoma tissue.

As an additional strategy to normalize for patient to patient variation in the expression levels of IBABP and IBABP-L, we compared their ratio of expression in colon cancer. In this instance the ratio of IBABP-L/IBABP in colorectal cancer (R_{C}) was compared to the ratio of expression in adjacent normal tissue (R_{N}) (FIG. 11B). In colorectal cancers R_{C} differed significantly from adjacent normal tissue R_{N} (P<0.0001). Using two-fold cut-off, the R_{C}/R_{N} ratio increases in 78% (53/68) of colorectal cancers, indicating this measurement is a better predictive tool for identifying malignant tissue.

The IBABP-L protein is expressed and up-regulated in colorectal cancer. Studies were conducted to determine if the IBABP-L protein is expressed in colon tissue or in colon cancer. First, antiserum against IBABP-L was raised in rabbits by immunization with the peptide CTWVSRKGDLQRMKQTHKGKPPSS (SEQ ID NO: 16), a peptide within the 49-residue N-terminal sequence found in IBABP-L. The antiserum from immunized animals was tested for reactivity against recombinant IBABP-L and IBABP by Western blot. Recombinant IBABP-L and IBABP were separated on SDS-PAGE, transferred to nitrocellulose and probed with the antiserum (1:2000 dilution). The Western blot was probed with antiserum against IBABP-L peptide or with antiserum raised against recombinant IBABP. The specificity of the anti-serum against IBABP-L was confirmed by competition with soluble peptide antigen. In this case, 2 μL of antisera was incubated with either 1 or 10 μg of antigen prior to use in Western blot. The antiserum was found to be highly specific for IBABP-L and without binding to IBABP. Furthermore, binding of the antiserum to IBABP-L could be blocked by competition with soluble peptide antigen.

The expression of IBABP-L protein was also measured in human colorectal carcinoma by immunohistochemistry using an IBABP-L selective antiserum. Paraffin-embedded slides of human colorectal carcinoma and its adjacent normal tissue were stained by rabbit antiserum raised against an IBABP-L selective peptide, followed by a diaminobenzidine-based detection method employing horseradish peroxidase system. Human fetal colon slide (Biochain) and normal adult ileum (Biospring) was also stained the same way. In some cases epithelial cells at the apex of the villi show weak staining for IBABP-L. However, the vast majority of epithelial cells in villi and crypts lack staining. In contrast, nearly all cancer cells were stained positively. The staining appeared to be independent of the differentiation status of the tumor. Interestingly, fetal ileum was also stained by antiserum against IBABP-L. Fetal epithelial cells all expressed IBABP-L, but this expression was lost in most adult epithelial cells. Importantly, adult ileum lacks expression of IBABP-L. This finding in contrast to the fact that IBABP is expressed to high levels in the ileum (Fujita et al., Eur. J. Biochem. 233:406-413, 1995; Grober et al., J. Biol. Chem. 274:29749-29754, 1999).

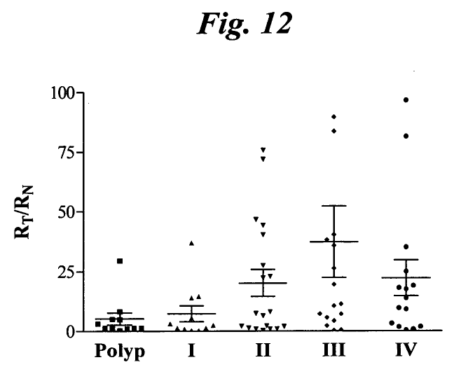

Effects of tumor stage on the expression of IBABP-L and the ratio of IBABP-L/IBABP. Experiments were also conducted to determine if tumor stage significantly affects the expression of IBABP-L. We found an obvious trend toward increased expression of the mRNA encoding IBABP-L as a function of tumor stage, although this trend was not entirely statistically significant. Therefore, we explored whether patient-to-patient variance in IBABP-L could be normalized by dividing by the level of IBABP expression. The ratio of IBABP-L/IBABP was calculated for tumor tissue (RT) and for normal adjacent tissue (R_{N}). As illustrated in FIG. 12, there is a trend of increasing R_{T}/R_{N} across the first four stages of colon cancer (polyps to stage III). Although the differences are not significant between polyps and stage I, nor from stage II to stage IV, the difference between these two groups is statistically significant. The ratio of IBABP-L/IBABP in cancers was also independent of patient age, gender, differentiation level, and tumor locale.

IBABP-L is expressed in colon carcinoma cells lines that contain distinct oncogenic lesions. It is now clear that colorectal cancer can be initiated by a number of genetic lesions including the mutations and deletions in the tumor suppressor genes DCC, APC and p53 along with mutations in the oncogene K-ras. Experiments were conducted with colon cancer cell lines to determine if the type of oncogenic lesions influenced the ratio of expression between IBABP-L and IBABP. These studies were performed in colon cancer cell lines which contain distinct lesions (Table 1 below). The ratio of IBABP-L/IBABP in seven cell lines is more than 2.0 (from 2.17 to 19.65), only SW480 has a ratio less than 2.0 (1.42). Consequently, the type of oncogenic lesion has little effect on the ratio of IBABP-L to IBABP. These observations indicate that an assessment of the expression of IBABP-L is likely to be applicable in the detection of colon cancers arising from a broad range of oncogenic lesions.

### Discussion

We have identified a new variant of IBABP and designated it as IBABP-L. The transcript for IBABP-L arises from an alternative start site and includes three exons that are absent in IBABP. IBABP-L also shares part of a fourth exon with IBABP. The protein encoded by IBABP-L contains a deduced 49 residue N-terminal sequence that is not found in IBABP. The IBABP-L transcript is expressed at similar levels throughout the normal human intestine. This is in contrast to the transcript encoding IBABP, which is expressed at levels several orders of magnitude higher in the section of the intestine extending from the jejunum to the ascending colon. In these regions of the intestine, the expression of IBABP-L is at least an order of magnitude lower than IBABP. The two transcripts also differ in their response to bile acids. While bile acids stimulate the expression of IBABP as part of the FXR transcription pathway (Grober et al., J. Biol. Chem. 274:29749-29754, 1999), they are without effect on the expression of IBABP-L.

IBABP was recently reported to be up-regulated in colorectal cancer in conjunction with a decrease in the expression of FXR (DeGottardi et al., Dig. Dis. Sci. 49:982-989, 2004). However, that study was performed prior to our discovery of IBABP-L, and did not distinguish between the two forms of IBABP. Here, we compared the expression of IBABP and IBABP-L in colorectal carcinoma samples from 68 patients. We report that IBABP remains essentially unchanged in colorectal cancer, but that its alternative transcript, IBABP-L, is up-regulated. In most cases the up-regulation is substantial, with the mean increase in relative mRNA copy number being greater than 30-fold. IBABP-L is up-regulated in early malignant polyps and its high expression is evident in all subsequent clinical classifications of tumor differentiation. Although a trend toward up-regulation in colorectal cancer is evident with PCR primers that fail to distinguish between the two transcripts, a specific measure of IBABP-L is far more sensitive.

Three other factors are important to consider in the use of IBABP-L as a biomarker. First, the increase in IBABP-L expression in colorectal cancer is independent of the patients' age or gender. Second, based on studies in colon cancer cells lines, the expression of IBABP-L appears to be independent of common oncogenic mutations to proteins like p53, APC, or K-ras. Any subtle links between IBABP-L and these oncogenic mutations will be best studied in larger more comprehensive analysis of tumor samples from patients. Nevertheless, in conjunction with the fact that IBABP-L is up-regulated in most tumors, the studies from cell lines show that it is highly unlikely that the expression of IBABP-L is dependent on a lesion in a single oncogene. Third, unlike IBABP, the expression of IBABP-L is not influenced by bile acids. Therefore, one would not expect the levels of IBABP-L to be tied to changes in bile acids resulting from dietary changes or overall health status. Collectively, the expression of IBABP-L has many properties that make it well suited for use as a broadly applicable test for colorectal cancer.

As with most studies comparing biomarker levels across populations of patients we used a normalization procedure. In this study we chose to use acidic ribosomal phosphoprotein (ARPP0) as a normalization standard because it is rather widely accepted for normalization in studies of gene expression in cancer (Chene et al., Int. J. Cancer 111:798-804, 2004; Cacev et al., Gut 54:1129-1135, 2005), and because our preliminary analysis indicated that this gene had the most consistent expression levels in colorectal tumors. However, there are other “housekeeping” genes that could be used for normalizing the expression levels of IBABP-L. We have conducted a small survey of tumor samples to gauge the applicability of other normalization standards, like Cyclophilin A, GADPH, and β-actin. Interestingly, when β-actin was used for normalization of IBABP-L, the assay detected tumors that were missed when ARPP0 was used. In fact, in sixteen tumors where the change in IBABP-L normalized to ARPP0 was less than two-fold, nine showed greater than a two-fold increase in IBABP-L when normalized to β-actin. We chose not to use β-actin as a normalization standard in the analysis of all sixty-eight tumors because β-actin levels are reported to change in colon cancer (Khimani et al., Biotechniques 38:739-745, 2005). As another approach toward normalization we also calculated the ratio of expression between IBABP-L and IBABP (R_{C}/R_{N}) in samples, and we found this ratio to be a slightly better predictor of colorectal cancer than the relative levels of IBABP-L alone.

The expression of IBABP-L and its up-regulation in colon cancer are likely to impact our understanding of the role of secondary bile acids in the onset and progression of colon cancer. Although insufficient to initiate oncogenesis alone, secondary bile acids strongly promote tumorigenesis (Bernstein et al., Mutat. Res. 589:47-65, 2005). IBABP-L may be initially up-regulated as a defense mechanism against secondary bile acid-mediated apoptosis. Increased levels of IBABP-L would allow more binding of bile acids, decreasing cellular bile acid concentration and thus lessen contact with carcinogens. A protective buffer from bile acid damage may at first create a cellular growth advantage. However, an oncogenic program of uncontrolled cell growth: progression from hyperplasia to a final invasive phenotype, may later supplant any original benefit. The mechanism of this action remains unclear, however, raising the possibility that up-regulation of IBABP-L ultimately indicates participation as a signaling molecule in an as-yet-unknown pro-oncogenic pathway.

In summary, we observed significant differences in the transcription of IBABP-L between normal colon tissue and colon cancer. Statistically significant differences in the expression of IBABP-L are evident in all stages of colon cancer, ranging from polyps to Stage 1V colorectal cancer. Therefore, IBABP-L is an especially exciting biomarker for colon cancer.

### EXAMPLE 2

### Reducing Levels of IBABP-L as a Treatment for Colorectal Cancer

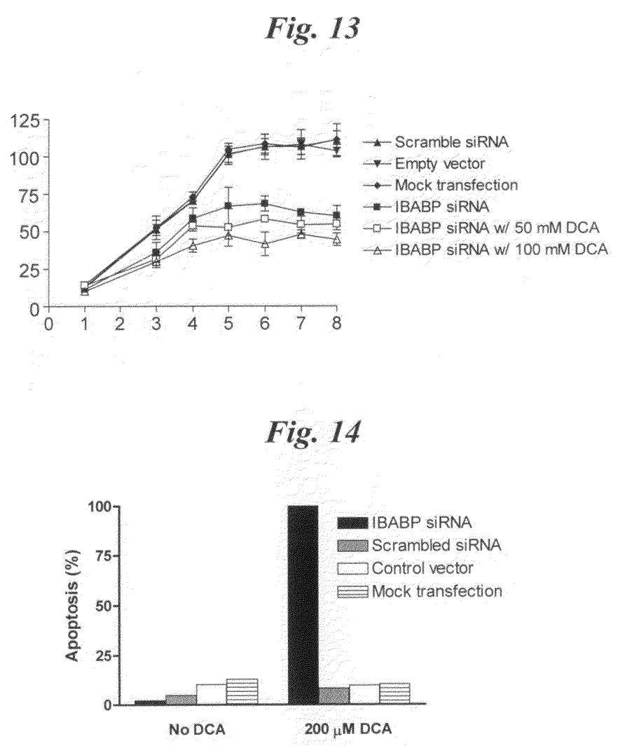

IBABP-L is involved in growth of colorectal cancer cells. We have demonstrated that IBABP-L is also involved in growth of colorectal cancer cells. FIG. 13 shows that a reduction in the expression of IBABP-L with shRNA inhibits the growth of HCT 116 cells. The growth of HCT 116 colorectal cancer cells was monitored over an eight-day period. Cells were seeded into the wells of a 96-well microtiter plate at a density of 7000 cells per well. Growth was monitored daily the Promega CellTiter kit. Each day twenty micro liters of this reagent was added to wells and incubated at 37° C. for 1.5 hr. The colorimetric reading of wells was recorded at 490 nm. Cell growth is plotted as a percentage of the absorbance of the initial seeding (7000 cells/well). To gauge the effects of knock-down of IBABP-L on growth, cells were transfected with the pSM2c retroviral vector encoding an shRNA targeting IBABP-L. Controls include cells that underwent a mock transfection, cultures of cells transfected with an empty vector, and cells transfected with an irrelevant siRNA. When HCT 166 colon cancer cells, growing in log-phase, were transfected with a vector encoding an shRNA that knocks down the expression of IBABP-L, the relative growth rate was reduced by 35% (P<0.001). The effect of the knock-down of IBABP-L on cell growth was even more pronounced when the cells were grown in the presence of deoxycholic acid (DCA), a secondary bile acid. These observations show that a reduction in the expression of IBABP-L reduces the growth of colon cancer cells. A reduction in intracellular levels of this protein is useful for treating colorectal cancer.

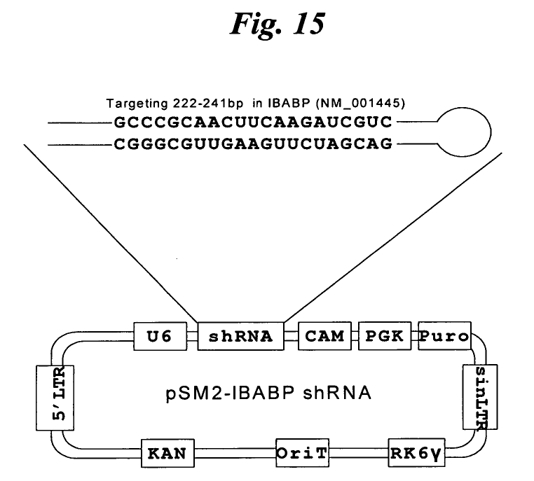

IBABP-L regulates the sensitivity of colorectal cancer cells to secondary bile acids. Ileal bile acid binding protein is also essential for protecting colon cancer cells from the toxic effects of secondary bile acids, like deoxycholic acid. As shown in FIG. 14, HCT116 cells were transfected with pSM2c retroviral vector encoding an shRNA targeting IBABP-L, with an irrelevant scrambled shRNA, with the empty vector, or simply subjected to a mock transfection. The construction of pSM2-IBABP shRNA is shown in FIG. 15. Cells were seeded at 2,000 cells/well in 96 well plates. At 48 hr, the cells were treated with either vehicle or with 200 μM deoxycholic acid for 24 hr. Cell death was monitored using the Cell Death Detection ELISAplus kit (Roche) according to manufacturer's protocol. This kit quantifies DNA fragmentation, a process unique to apoptosis. When HCT 116 colon cancer cells are treated with 200 uM of deoxycholic acid (DCA) the cells survived and showed very little apoptosis. However, when the same cells were transfected with a vector encoding an shRNA that targets IBABP-L, and were then treated with DCA, nearly all of the cells died from apoptosis. This finding indicates that IBABP-L is necessary for the survival of colon cancer cells in the presence of physiologic levels of secondary bile acids. Consequently, therapies aimed at reducing or eliminating the expression of IBABP-L in colon cancer cells are likely to elicit the death of such cells.

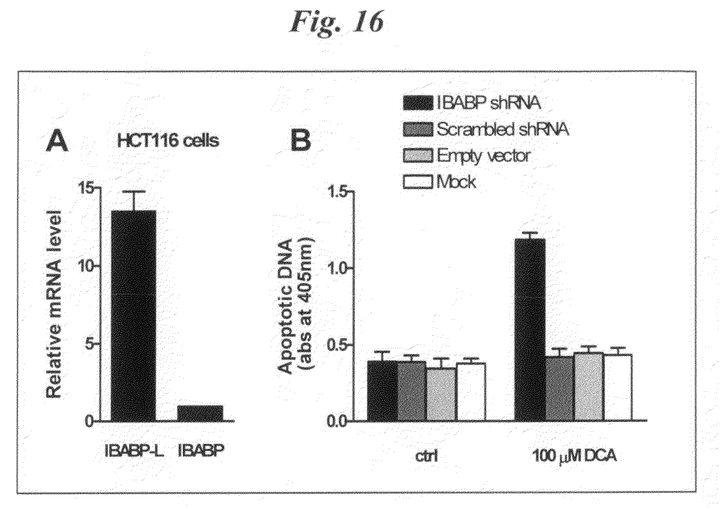

Additional studies were conducted to determine if IBABP-L has a role in conferring bile acid resistance in colon cancer cells. As a model system, we chose the HCT 116 colon cancer cell line because it expresses high levels of IBABP-L, but barely detectable levels of IBABP (FIG. 16A). Therefore, this cell line recapitulates the expression pattern of IBABP-L and IBABP that we observe in human colon cancer tissue. RNA interference was used to knock down the expression of IBABP-L in HCT116 cells. shRNA encoding constructs were purchased from Open Biosystems (Huntsville, Ala.). Those constructs are in pSM2c vector and transcribed by type III RNA polymerase through U6 promoter, encoding IBABP or scrambled shRNA. HCT116 cell suspension were mock transfected, or transfected with either pSM2c-IBABP, pSM2c-scrambled, or pSM2c at 200 ng DNA/1000 cells and the seeded into 96-well plate at 1000 cells/well. Following transfection, cells were incubated at 37° C. for 72 h and treated with DCA in different concentration for 24 h. The cell apoptosis was measured using the Cell Death Detection ELISAplus kit (Roche Applied Science, IN), which detect the amount of cleaved DNA/histone complexes using a sandwich-enzyme-immunoassay-based method. The value was determined using a colorimetric 96 well plate reader (Bio-Rad, CA) and represented as mean±SD of three independent experiments performed in quadricate. Quantification of mRNA encoding IBABP-L showed that the expression was reduced by 50%, and that this level of repression was maintained four days. Two days after the knockdown of IBABP-L, the cells were incubated with 100 μM DCA for 24 h, and the number of cells undergoing apoptosis was determined. The level of apoptosis in cells where IBABP-L was knocked down was substantially higher than that in cells transfected with scrambled shRNA, or other control groups (FIG. 16B). We found nearly identical results when activation levels of caspase 8 and caspase 9 were analyzed. This observation is consistent with reports in the literature showing that DCA activates both caspases (Yui et al., J. Biochem. [Tokyo] 138:151-157, 2005).

In addition to the siRNA described above, several other siRNA sequences are expected to produce a knock-down of IBABP-L, and thereby reduce the growth rate and increase apoptosis in colon cancer cells. A few examples are shown in Table 2 below. These siRNA sequences were designed using the web-based tools called siRNA Target Finder (Ambion) and siRNA design Tool (Qiagen). Importantly, IBABP-L shares considerable identity with IBABP; therefore, si- and shRNA molecules targeting IBABP are also expected to knock down IBABP-L, and to exhibit the same biological effects on colon cancer cells.

### Discussion

In CRC, IBABP-L is up-regulated by NF-κB and this up-regulation appears to be necessary for resistance of colon cancer cells to bile acid-induced apoptosis. Together these findings suggest the existence of a bile acid response pathway controlled by NF-κB, and requiring IBABP-L that is essential for the survival of colon cancer cells in the presence of secondary bile acids.

Perhaps the most important distinction between IBABP and IBABP-L is the way in which their expression is regulated. IBABP is part of the FXR transcription pathway (23) that responds to bile acids and regulates their reabsorption across the ileum. In contrast, IBABP-L is not regulated by FXR; it is regulated by NF-κB. One of the paradoxical findings in the literature on IBABP is the observation that it was up-regulated in CRC while its key regulator, FXR is down-regulated (14). The discovery that the seeming up-regulation of IBABP is actually up-regulation of IBABP-L, and the fact that this transcript is regulated by NF-κB, resolve this paradox.

In addition to the differential expression and regulation of IBABP-L, we provide a biological function for IBABP-L in colon tumorigenesis; IBABP-L is necessary for the survival of colon cancer cells in the presence of physiological levels of DCA, a toxic secondary bile acid (27). The concentration of DCA in the fecal water of normal subjects is approximately 100 μM (3). We observed that colon cancer cells are resistant to cell death induced by these levels of DCA; however, when the expression of IBABP-L is knocked down, the cells undergo apoptosis. IBABP-L is also necessary for survival of cells in the presence of concentrations of DCA observed in colorectal cancer patients, approximately two-fold higher than normal levels (28). These observations show that IBABP-L promotes survival of CRC cells in the presence of toxic bile acids and is likely to contribute to colon tumorigenesis.

The intriguing link between bile acids, NF-κB and IBABP-L may provide a novel target for intervention in colorectal cancer. In conjunction with the findings in the literature, our study indicates that IBABP-L is up-regulated as a result of the constitutive activation of NF-κB in CRC (29, 30). This coupling of NF-κB and IBABP-L enables colon cancer cells to buffer toxic bile acids, protecting the cells from apoptosis. IBABP-L can be exploited as a therapeutic target in two ways. First, inhibitors of IBABP-L may enhance the chemopreventative and therapeutic effects of NF-κB inhibitors (31). Second, because IBABP-L is necessary for tumor cell survival in the presence of bile acids, its inhibition would be expected to cause tumor cell death in the colon.

### EXAMPLE 3

### Use of the IBABP-L Promoter to Express Therapeutically Useful Genes in Colorectal Cancer Cells

The promoter region of IBABP-L as a DNA regulatory element that can be used to drive the expression of therapeutically useful genes in colon cancer cells. Since our prior patent showed the up-regulation of IBABP-L by 50-fold (on average) in human colon cancer tissue, we suggest that this promoter can be used to overexpress proteins delivered by gene therapy. Here is a results section that describes our “functional dissection” of this promoter. This results show that one of the key transcription factors involved in driving IBABP-L expression is NF-κB. We should certainly claim this as a minimal motif, BUT should not limit ourselves to this short sequence and should claim the entire promoter to −1568 (prior to start site) as this region is likely to contain additional regulatory sites.

Transcription of IBABP-L is controlled by NF-κB. As a first step toward understand the transcription regulation of IBABP-L, primer extension was performed to identify the transcription start site (TSS). This information provided an anchor point for mapping regions of the promoter of IBABP-L. The genomic sequence of IBABP-L extending 1.6 Kb upstream of the TSS was analyzed for putative transcription factor binding sites using the P-Match program (http://www.gene-regulation.com).

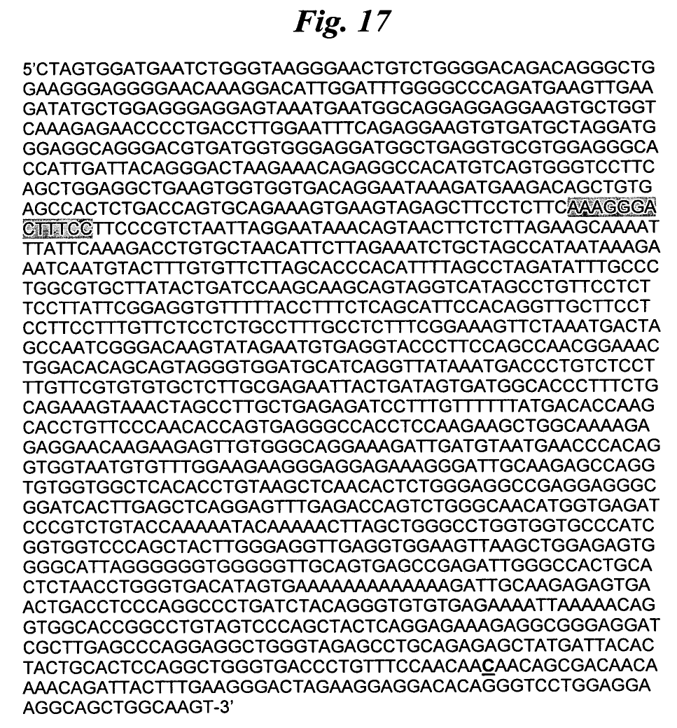

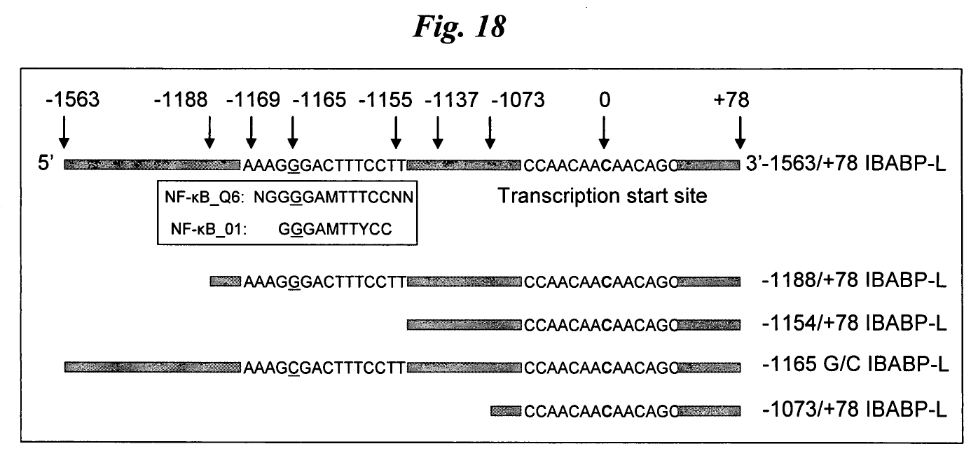

FIG. 17 shows the nucleotide sequence of the IBABP-L promoter (from nucleotides −1563 to +78). A putative NF-κc binding motif was identified 1.16 Kb upstream of the TSS, starting at nucleotide position −1169 and ending at −1153 (FIGS. 17 and 18).

To verify that NF-κB binds the response element and drives the expression of IBABP-L, a number of studies were performed with deletions and mutations of the promoter linked to a reporter gene (PGL3-Basic Luciferase), as shown in FIG. 18. These studies were performed in HCT 116 colon cancer cells, which have elevated expression of IBABP-L. The region containing the putative NF-κB binding motif (−1563/+78) was amplified from human genomic DNA by PCR. Deletion constructs of this promoter were made through PCR or restriction enzyme digestion. The G C mutation was made through site-directed mutagenesis. The wild type and modified promoters were inserted into PGL3 vector containing firefly Luciferase (Luc) reporter (Promega). Reporter vectors were constructed to either delete the entire putative NF-κB binding motif, or mutate the most conserved “G” to “C” within this motif. The wild type promoter and modified reporters were introduced into HCT 116 cells, and luciferase activity was analyzed at 24 hr.

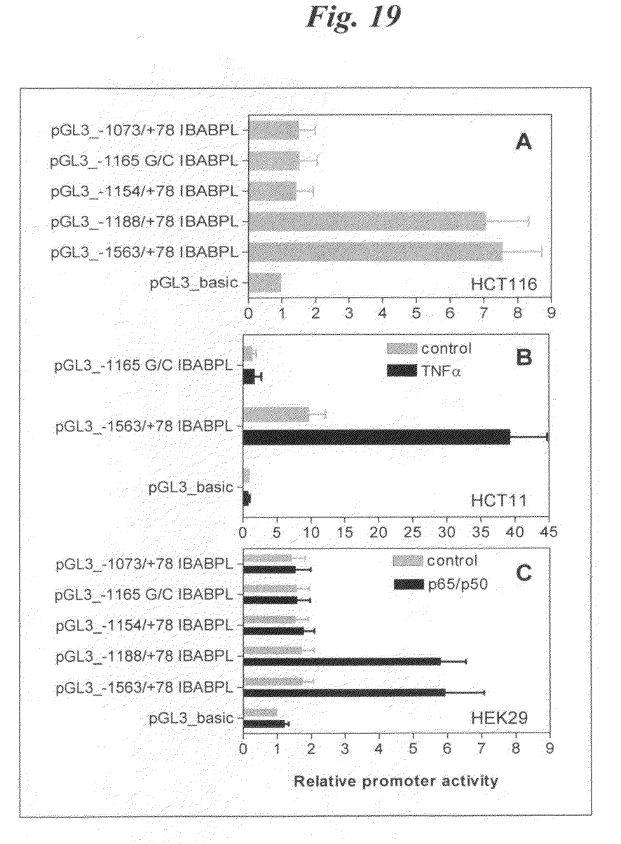

As shown in FIGS. 19A-C, a functional analysis of the IBABP-L promoter was performed. In FIG. 19A, reporter constructs were introduced into HCT 116 cells together with pRL-CMV which encodes Renilla luciferase (FIG. 19A). The luciferase activity was measured in 24 h and normalized to the Renilla signal in the absence of the IBABP-L promoter. FIG. 19B shows the results of experiments that are similar to those shown in FIG. 19A except that the transfected HCT116 cells were treated with or without 25 ng/ml TNFα for 5 hr before measurement. FIG. 19C shows the results of experiments in which wild type and modified IBABP-L promoter activity reporter constructs were introduced into HEK293 cells with or without co-transfection with constructs encoding NF-κB complex p65/p50. Luciferase activity was measured in 24 hr and normalized. pRL-CMV was included in every transfection. The wild type promoter for IBABP-L produced a 7.5 fold increase in luciferase activity compared to the basic reporter (FIG. 19A). Deletion of the sequences upstream of the putative NF-Kb binding motif had no effects on the luciferase activity. However, when the whole binding motif was deleted by either PCR or restriction enzyme digestion, the reporter constructs nullified its transcriptional activity. More importantly, a single nucleotide substitution from “G” to “C” at the most conserved region of the NF-Kb binding motif also resulted in loss of promoter activity (FIG. 19A). Furthermore, tumor necrosis factor α (TNFα), which is an activator of NF-Kb, increased the transcription activity of the wild type promoter, but not the mutated promoter (FIG. 19B).

The activity of the IBABP-L promoter was also tested in HEK293 cells, which have low endogenous levels of NF-Kb. In these cells the IBABP promoter had low activity (FIG. 18B), but its activity increased when constructs encoding protein p65 and p50 of NF-Kb were co-transfected into HEK293 cells (FIG. 19C). This activation by p65/p50 was not observed when the IBABP-L promoter lacking a functional NF-Kb binding site was tested (FIG. 19B). Together, these findings strongly support the idea that NF-Kb regulates the transcription activity of the IBABP-L promoter.

Discussion. In addition to the differential expression and regulation of IBABP-L, we provide a biological function for IBABP-L in colon tumorigenesis; IBABP-L is necessary for the survival of colon cancer cells in the presence of physiological levels of DCA, a toxic secondary bile acid (Nagengast et al., Eur. J. Cancer 31:1067-1070, 1995). The concentration of DCA in the fecal water of normal subjects is approximately 100 μM (Bernstein et al., Mutat. Res. 589:47-65, 2005). We observed that colon cancer cells are resistant to cell death induced by these levels of DCA; however, when the expression of IBABP-L is knocked down, the cells undergo apoptosis. IBABP-L is also necessary for survival of cells in the presence of concentrations of DCA observed in colorectal cancer patients, approximately two-fold higher than normal levels (Reddy et al., Cancer Res. 35:3403-3406, 1975). These observations show that IBABP-L promotes survival of CRC cells in the presence of toxic bile acids and is likely to contribute to colon tumorigenesis.

The intriguing link between bile acids, NF-κB and IBABP-L provides a novel target for intervention in colorectal cancer. In conjunction with the findings in the literature, our study indicates that IBABP-L is up-regulated as a result of the constitutive activation of NF-κB in colorectal cancer (Shah et al., Int. J. Cancer 118:532-539, 2006; Shishodia and Aggarwal, J. Biol. Chem. 279:47148-47158, 2004). This coupling of NF-κB and IBABP-L enables colon cancer cells to buffer toxic bile acids, protecting the cells from apoptosis. IBABP-L can be exploited as a therapeutic target in two ways. First, inhibitors of IBABP-L may enhance the chemopreventative and therapeutic effects of NF-κB inhibitors (Gilmore and Herscovitch, Oncogene 25:6887-6899). Second, because IBABP-L is necessary for tumor cell survival in the presence of bile acids, its inhibition would be expected to cause tumor cell death in the colon.

All publications, patents and patent applications are incorporated herein by reference. While in the foregoing specification, this invention has been described in relation to certain embodiments thereof, and many details have been set forth for purposes of illustration, it will be apparent to those skilled in the art that the invention is susceptible to additional embodiments and that certain of the details herein may be varied considerably without departing from the basic principles of the invention.

TABLE 1
Genetic lesions in colorectal cancer cell lines

| | APC | TP53 | K-ras | Stage | Duke's type | IBABP-L/IBABP |
|---|---|---|---|---|---|---|
| Caco-2 | mutant | mutant | mutant | II | B | 2.17 ± 0.07 |
| SW480 | mutant | mutant | mutant | n/a* | B | 1.42 ± 0.07 |
| HCT116 | wild type | wild type | mutant | n/a* | n/a* | 15.10 ± 2.40 |
| LS 174T | wild type | wild type | mutant | n/a* | B | 19.65 ± 1.65 |
| LoVo | mutant | wild type | mutant | IV | C | 6.07 ± 0.66 |
| SW403 | mutant | wild type | mutant | III | C | 13.25 ± 0.65 |
| WiDr^{#} | n/a* | mutant | wild type | n/a* | n/a* | 10.5 ± 0.20 |
| HT-29 | mutant | mutant | wild type | I | n/a* | 3.47 ± 0.34 |

^{#}DNA fingerprinting has shown this line to be a derivative of HT-29.
*Not available from literature.

TABLE 2
siRNA sequences for reducing levels of IBABP or IBABP-L

| | SEQ ID | | SEQ ID | |
|---|---|---|---|---|
| | NOS | IBABP-L specific siRNA | NOS | IBABP specific siRNA |
| 1 | 29 | 5′-UGAAACAGACACAUAAAGGUU-3′ | 35 | 5′-CUGGGAGAGUUUAUAAGCUUU-3′ |
| | 30 | 3′-UUACUUUGUCUGUGUAUUUCC-5′ | 36 | 3′-UUGACCCUCUCAAAUAUUCGA-5′ |
| 2 | 31 | 5′-AGGAGACCUGCAGAGAAUGUU-3′ | 37 | 5′-AGCAACUGGGAGAGUUUAUUU-3′ |
| | 32 | 3′-UUUCCUCUGGACGUCUCUUAC-5′ | 38 | 3′-UUUCGUUGACCCUCUCAAAUA-5′ |
| 3 | 33 | 5′-GACAGUGACGAUGAUGAUGUU-3′ | 39 | 5′-GAGAGUUUAUAAGCUGGGAUA-3′ |
| | 34 | 3′-UUCUGUCACUGCUACUACUAC-5′ | 40 | 3′-CUCUCAAAUAUUCGACCCUAU-5′ |

What is claimed is:

1. A method of reducing the growth or survival of a colorectal cancer cell comprising contacting the cell with an effective amount of a composition comprising a substance that reduces bile-acid binding by IBABP-L.

2. The method of claim 1 wherein the substance reduces IBABP-L polypeptide levels in the cell without reducing IBABP polypeptide levels.

3. The method of claim 2 wherein the substances inhibits IBABP-L gene expression.

4. The method of claim 3 wherein the substance is a polynucleotide.

5. The method of claim 4 wherein the polynucleotide is a member of the group consisting of an siRNA, an antisense polynucleotide, and a ribozyme.

6. The method of claim 5 wherein the substance is an siRNA.

7. The method of claim 4 wherein the polynucleotide comprises a promoter that is expressible in the colorectal cancer cell and that is operably linked to a sequence encoding a a polynucleotide that, when expressed, reduces levels of a polypeptide selected from the group consisting of IBAB-L, a enzyme of metabolism, a protein essential for cell-cycle progression, a protein that inhibits apoptosis, a protein involved in growth regulatory signal transduction, and a protein involved in bile acid transport out of colorectal cancer cells.

8. The method of claim 4 wherein the polynucleotide comprises a promoter that is expressible in the colorectal cancer cell and that is operably linked to a sequence encoding a member of the group consisting of an siRNA, an antisense polynucleotide and a ribozyme.

9. The method of claim 4 wherein the polynucleotide comprises a promoter that is expressible in the colorectal cancer cell and that is operably linked to a sequence encoding a polypeptide selected from the group consisting of a pro-apoptotic protein, a tumor suppressor protein, a protein that inhibits cell cycle progression, and a protein involved in the delivery of toxic secondary bile acids into the cytoplasm of colorectal cancer cells.

10. The method of claim 3 wherein the substance inhibits transcriptional activation of IBABP-L gene expression.

11. The method of claim 1 wherein the composition comprises a member of the group consisting of a bile acid, a chemotherapeutic drug, a non-steroidal anti-inflammatory drug; a vaccine comprising autologous tumor cells, a vaccine comprising a tumor-associated antigen, an monoclonal antibody directed against a tumor antigen, a recombinant construct for gene correction, a virus-directed enzyme-prodrug treatment, and a matrix metalloproteinase inhibitor.

12. The method of claim 11 wherein the composition comprises a bile acid selected from the group consisting of cholic acid and deoxycholic acid.

13. The method of claim 1 wherein the composition causes apoptosis of the colorectal cancer cell in the presence of a bile acid.

14. A method of treating colorectal cancer in a patient in need of such treatment comprising administering to the patient an effective amount of a composition that reduces bile-acid binding by IBABP-L.

15. A composition comprising a polynucleotide selected from the group consisting of: an expression vector comprising an IBABP-L promoter operably linked to a sequence that, when expressed, reduces bile-acid binding by IBABP-L; an siRNA that reduces IBABP-L gene expression; an antisense polynucleotide that reduces IBABP-L gene expression; and a ribozyme that reduces IBABP-L gene expression.

16. The composition of claim 15 comprising the expression vector, wherein the sequence encodes a polypeptide selected from the group consisting of: a pro-apoptotic protein; a tumor suppressor; an inhibitor of cell cycle progression; a protein involved in the delivery of toxic secondary bile acids into the cytoplasm of colorectal cancer cells; and an inhibitor of transcriptional activation of IBABP-L gene expression.

17. The composition of claim 16 comprising the expression vector, wherein the sequence encodes a polynucleotide that reduces levels of a polypeptide in the colorectal cancer cell, wherein the polypeptide is selected from the group consisting of IBAB-L, a enzyme of metabolism, a protein essential for cell-cycle progression, a protein that inhibits apoptosis, a protein involved in growth regulatory signal transduction, and a protein involved in bile acid transport out of colorectal cancer cells.

18. The composition of claim 17 comprising the expression vector wherein the sequence encodes a polynucleotide selected from the group consisting of an siRNA, an antisense polynucleotide, and a ribozyme.

19. The composition of claim 15 comprising the expression vector wherein the sequence, when expressed in a colorectal cancer cell that is in the presence of a bile acid, causes apoptosis of the colorectal cancer cell.

20. The composition of claim 15 comprising a carrier.

21. The composition of claim 15 further comprising, in addition to the polynucleotide, at least one active ingredient that inhibits bile-acid binding activity of IBABP-L.

22. The composition of claim 21 wherein said at least one active ingredient is selected from the group consisting of a chemotherapeutic drug, a non-steroidal anti-inflammatory drug, a vaccine comprising autologous tumor cells, a vaccine comprising a tumor-associated antigen, a monoclonal antibody directed against a tumor antigen, a recombinant construct for gene correction, a virus-directed enzyme-prodrug treatment, and a matrix metalloproteinase inhibitor.

23. The composition of claim 15 that is effective for treating colorectal cancer in a patient in need of such treatment.

24. A method of treating colorectal cancer in a patient in need of such treatment comprising administering to the patient an effective amount of the composition of claim 15.

25. A composition for treating colorectal cancer in a patient in need of such treatment comprising an effective amount of an siRNA construct that reduces levels of IBABP-L polypeptide in the patient.

26. The composition of claim 25 that reduces levels of IBABP-L polypeptide in the patient without substantially reducing levels of IBABP polypeptide.

27. The composition of claim 25 wherein the composition further comprises a bile acid.

28. The composition of claim 27 wherein the bile acid is selected from the group consisting of cholic acid and deoxycholic acid.

29. The composition of claim 25 wherein the composition comprises a pharmaceutically acceptable carrier.

30. A method of treating colorectal cancer in a patient in need of such treatment comprising administering to the patient an effective amount of a composition of claim 25.

31. The method of claim 30 comprising administering the composition to the patient orally or rectally.

32. The method of claim 31 comprising administering the composition to the patient rectally by enema.

33. A method for identifying an agent that is effective in reducing the growth or survival of a colorectal cancer cell comprising: (a) contacting a sample comprising IBABP-L polypeptide with a composition comprising the agent; and (b) determining whether the composition reduces bile-acid binding activity by the IBABP-L polypeptide.

34. The method of claim 33 wherein the sample is a colorectal cancer cell.

35. The method of claim 34 comprising determining whether the composition reduces levels of IBABP-L polypeptide in the colorectal cancer cell.

36. The method of claim 33 comprising contacting the colorectal cancer cell with the composition and determining whether the composition reduces growth or survival of the colorectal cancer cell.
